SEQUENCE LISTING (41 sequences)

SEQ ID NO: 1 - DNA, 636 bp, Artificial Sequence
  synthetic construct
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 2 - protein, 211 aa, Artificial Sequence
  synthetic construct
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 3 - DNA, 30 bp, Artificial Sequence
  synthetic construct
aactccctcgatgtggcggctcatctgccc

SEQ ID NO: 4 - DNA, 31 bp, Artificial Sequence
  synthetic construct
aactccctcgaatgtggcggctcatctgccc

SEQ ID NO: 5 - DNA, 29 bp, Artificial Sequence
  synthetic construct
tctcctggctcagagggagctcgaggctg

SEQ ID NO: 6 - DNA, 29 bp, Artificial Sequence
  synthetic construct
  misc_feature (5)..(11): n is a, c, t, g or can be none
tctcnnnnnnnagagggagctcgaggctg

SEQ ID NO: 7 - DNA, 28 bp, Artificial Sequence
  synthetic construct
ctggttcctcagtcacacatgccagagg

SEQ ID NO: 8 - DNA, 26 bp, Artificial Sequence
  synthetic construct
cctctgagccaggagacattttcagg

SEQ ID NO: 9 - DNA, 26 bp, Artificial Sequence
  synthetic construct
ttccctcagccattgcctgtgtgtgg

SEQ ID NO: 10 - protein, 10 aa, Artificial Sequence
  synthetic construct
LVPQSHMPEV

SEQ ID NO: 11 - protein, 10 aa, Artificial Sequence
  synthetic construct
  MISC_FEATURE (4)..(4): Xaa can be any naturally occurring amino acid, or none
LVPXSHMPEV

SEQ ID NO: 12 - protein, 9 aa, Artificial Sequence
  synthetic construct
PLSQETFSG

SEQ ID NO: 13 - protein, 9 aa, Artificial Sequence
  synthetic construct
  MISC_FEATURE (4)..(4): Xaa can be any naturally occurring amino acid, or none
PLSXETFSG

SEQ ID NO: 14 - protein, 9 aa, Artificial Sequence
  synthetic construct
FPQPLPVCG

SEQ ID NO: 15 - protein, 9 aa, Artificial Sequence
  synthetic construct
  MISC_FEATURE (3)..(3): Xaa can be any naturally occurring amino acid, or none
FPXPLPVCG

SEQ ID NO: 16 - DNA, 34 bp, Artificial Sequence
  synthetic construct
ataacttcgtatagcatacattatacgaagttat

SEQ ID NO: 17 - DNA, 34 bp, Artificial Sequence
  synthetic construct
ataacttcgtataatgtatgctatacgaagttat

SEQ ID NO: 18 - DNA, 34 bp, Artificial Sequence
  synthetic construct
gaagttcctattctctagaaagtataggaacttc

SEQ ID NO: 19 - DNA, 48 bp, Artificial Sequence
  synthetic construct
gaagttcctatactttctagagaataggaacttcggaataggaacttc

SEQ ID NO: 20 - DNA, 12 bp, Artificial Sequence
  synthetic construct
ggatgagtcatc

SEQ ID NO: 21 - DNA, 10 bp, Artificial Sequence
  synthetic construct
gatgactcat

SEQ ID NO: 22 - DNA, 12 bp, Artificial Sequence
  synthetic construct
gatgagtcatcc

SEQ ID NO: 23 - DNA, 10 bp, Artificial Sequence
  synthetic construct
cctttgttcg

SEQ ID NO: 24 - DNA, 10 bp, Artificial Sequence
  synthetic construct
cccattgttc

SEQ ID NO: 25 - DNA, 16 bp, Artificial Sequence
  synthetic construct
cttggcactgtgccaa

SEQ ID NO: 26 - DNA, 10 bp, Artificial Sequence
  synthetic construct
gacatctggt

SEQ ID NO: 27 - DNA, 10 bp, Artificial Sequence
  synthetic construct
accatctgtt

SEQ ID NO: 28 - DNA, 12 bp, Artificial Sequence
  synthetic construct
gcggcagctgct

SEQ ID NO: 29 - DNA, 12 bp, Artificial Sequence
  synthetic construct
tatgcaaatgag

SEQ ID NO: 30 - DNA, 15 bp, Artificial Sequence
  synthetic construct
ccattgtatgcaaat

SEQ ID NO: 31 - DNA, 10 bp, Artificial Sequence
  synthetic construct
atttgcataa

SEQ ID NO: 32 - DNA, 15 bp, Artificial Sequence
  synthetic construct
atttgcataacaatg

SEQ ID NO: 33 - DNA, 15 bp, Artificial Sequence
  synthetic construct
ttgctttccaggaaa

SEQ ID NO: 34 - DNA, 8 bp, Artificial Sequence
  synthetic construct
ctgtttac

SEQ ID NO: 35 - DNA, 12 bp, Artificial Sequence
  synthetic construct
aggggatttccc

SEQ ID NO: 36 - DNA, 21 bp, Artificial Sequence
  synthetic construct
gcctcagccattgcctgtgtg

SEQ ID NO: 37 - DNA, 21 bp, Artificial Sequence
  synthetic construct
gcctcgagctccctctgagcc

SEQ ID NO: 38 - DNA, 21 bp, Artificial Sequence
  synthetic construct
gcagatgagccgccacatcga

SEQ ID NO: 39 - DNA, 20 bp, Artificial Sequence
  synthetic construct
cctcagccattgcctgtgtg

SEQ ID NO: 40 - DNA, 20 bp, Artificial Sequence
  synthetic construct
ctgagccaggagacattttc

SEQ ID NO: 41 - DNA, 20 bp, Artificial Sequence
  synthetic construct
tcctcagtcacacatgccag

(19) United States
(12) Patent Application Publication
(10) Pub. No.: US 2022/0304286 A1
(43) Pub. Date: Sep. 29, 2022

(54) SYSTEMS AND METHODS FOR IN VIVO DUAL RECOMBINASE-MEDIATED CASSETTE EXCHANGE (dRMCE) AND DISEASE MODELS THEREOF

(71) Applicant: CEDARS-SINAI MEDICAL CENTER, Los Angeles, CA (US)

(72) Inventors: Joshua Breunig, Beverly Hills, CA (US); Moise Danielpour, Los Angeles, CA (US); Gi Bum Kim, North Hollywood, CA (US); Alberto Ayala-Sarmiento, Hawthorne, CA (US); Amy Yang, Los Angeles, CA (US)

(73) Assignee: CEDARS-SINAI MEDICAL CENTER, Los Angeles, CA (US)

(21) Appl. No.: 17/617,878

(22) PCT Filed: Jun. 16, 2020
(86) PCT No.: PCT/US20/37946

§ 371 Date: Dec. 9, 2021

Related U.S. Application Data
(60) Provisional application No. 62/862,576, filed on Jun. 17, 2019.

(51) Int. Cl. A01K 67/027; C12N 15/10; C12N 15/86; C12N 15/90

(52) U.S. Cl. CPC A01K 67/0275; C12N 15/102; C12N 15/86; C12N 15/90; A01K 2217/052; A01K 2217/054; A01K 2217/15; A01K 2217/206; A01K 2227/105; A01K 2267/0331; C12N 2750/14143; C12N 2800/30; C12N 2800/40

(57) ABSTRACT

Described herein are donor vectors and systems for use in dual recombinase-mediated cassette exchange. Also described herein are animal models and human cells for consistent, rigorous, and facile investigation of transgene expression. Further described herein are methods of screening for therapeutic drugs using these animal models, and methods of treatment.

### CROSS-REFERENCE TO RELATED APPLICATIONS

This application includes a claim of priority under 35 U.S.C. § 119(e) to U.S. provisional patent application No. 62/862,576, filed Jun. 17, 2019, the entirety of which is hereby incorporated by reference.

### STATEMENT REGARDING FEDERALLY SPONSORED RESEARCH OR DEVELOPMENT

This invention was made with Government support under CA202900 and CA236687 awarded by the National Institutes of Health. The Government has certain rights in the invention.

### BACKGROUND

Genetically engineered mouse models (GEMMs) have been the paradigm for analyzing gene function in vivo in a temporal- and tissue-specific manner. However, as GEMM generation is an expensive laborious process, many alternative transgenic approaches, such as electroporation (EP)-mediated and viral gene deliveries, have been increasingly adapted as more rapid and efficient methods of creating somatic mosaics. Both methods entail injecting specific tissues with virus or foreign DNAs to transduce the surrounding cells and create somatic mosaics. EP can yield genome-inserted DNA using transposons or less efficiently with CRISPR/Cas9 and subsequent insertion of a donor template. Despite their speed, these methods have major pitfalls that dissuade more widespread adoption. Viral vectors have limited payloads, incite immune responses, and require special expertise, while both transposons and viral methods suffer from their unpredictable genomic integration patterns, possible insertional mutagenesis, and epigenetic transgene silencing. Both suffer from transgene copy number variability and overexpression artifacts such as cytotoxicity and transcriptional squelching, hence clonal genotypic/phenotypic variability are significant con-founding factors.

With the identification of hundreds of recurrent, putative cancer driver mutations, many of which are gain-of-function (GOF) oncogenes, it is imperative to create a tractable in vivo platform that can model these potential oncogenes, possibly in conjunction with tumor suppressor mutations. For each GOF oncogene, there are often tens of different recurrent missense mutations that can function in distinct ways. Many well-known tumor suppressor mutants are loss-of-function (LOF) phenotypes, for which one can utilize large-scale KO-mice consortia to breed multiple-KO-mice (e.g., Pten/p53/Nf1-KO). Even then, creating such mice is significantly time-consuming, expensive, and prone to some methodological confounds. Alternatively, CRISPR/Cas9 systems can simultaneously induce multiple KOs in vivo in mice, but can have significant unintended off-target genome alterations.

### SUMMARY OF THE INVENTION

In one aspect, provided herein is a flexible in vivo platform that can simultaneously model combinations of GOF and LOF mutations not only cheaply but also in a GEMM-like fashion. We demonstrate that successful dual recombinase mediated cassette exchange (dRMCE, or MADR) can be catalyzed in situ in somatic cells in well-characterized reporter mice with definitive genetic labeling of recombined cells. Moreover, we demonstrate the utility of this system in generating mosaicism with a mixture of GOF and LOF mutations, including patient-specific driver mutations. Ultimately, our MADR tumor models demonstrates this method has a potential to become a higher-throughput, first-pass experiment to test and study various putative tumor driver mutations, and provides a rapid pipeline for preclinical drug discovery in a patient-specific manner.

Described herein are systems, nucleic acids, and vectors useful for establishing a transgenic cell for use in cell therapy. These vectors circumvent problems associated with current methods used in creating cells with a transgene stably integrated in a genomic location. Current problems include lack of control of ploidy, lack of control of integration site, and restrictions on transgenic insert size. The systems described herein, solve these problems, and allow for safer more reproducible methods of cell therapy. These systems and the methods for using them are applicable to the establishment of cells and cell lines useful for delivering a gene product such as a neurotrophic factor and/or a growth factor to a subject with a neurodegenerative disease, such as Parkinson's disease, Amyotrophic Lateral Sclerosis (ALS), or Alzheimer's disease.

In one aspect, described herein, is a mammalian cell comprising a genomic integrated transgene, wherein the genomic integrated transgene comprises a neurotrophic factor, and is integrated at a genomic site comprising the AAVS1 locus, H11 locus, or HPRT1 locus. In certain embodiments, the cell is a human cell. In certain embodiments, the human cell is an induced pluripotent stem cell. In certain embodiments, the neurotrophic factor comprises glial cell line-derived neurotrophic factor (GDNF), neurturin, growth/differentiation factor (GDF) 5, mesencephalic astrocyte-derived neurotrophic factor (MANF), cerebral dopaminergic neurotrophic factor (CDNF), or combinations thereof. In certain embodiments, the neurotrophic factor is GDNF. In certain embodiments, the neurotrophic factor is under the control of an inducible promoter. In certain embodiments, the inducible promoter is a tetracycline or doxycycline inducible promoter. In certain embodiments, the neurotrophic factor and/or the inducible promoter are flanked by one or more of a recombinase recognition site, a tandem repeat of a transposable element, or an insulator sequence. In certain embodiments, a single copy of the transgene is integrated into the genome of the cell. In various embodiments, the neurotropic factor and/or the inducible promoter are flanked by paired recombinase recognition sites. In various embodiments, the paired recombinase recognition sites comprise a variant recombinase recognition site and a wild-type recombinase recognition site. In various embodiments, the variant recombinase recognition site exhibits reduced cleavage by a recombinase compared to the wild-type recombinase recognition site. In various embodiments, the paired recombinase recognition sites comprise LoxP sites or FRT sites.

In another aspect described herein is a system, comprising: (a) a promoter-less donor vector, comprising a polyadenylation signal or transcription stop element upstream from a transgene or nucleic acid encoding an RNA, the transgene or nucleic acid encoding an RNA, and paired recombinase recognition sites; (b) and one expression vector, comprising two genes encoding recombinases specific to the paired recombinase recognition sites, or two expression vectors, the first expression vector comprising one gene encoding a first recombinase that is specific to one of the paired recombinase recognition sites, and the second expression vector comprising one gene encoding a second recombinase that is specific to the other of the paired recombinase recognition sites. In certain embodiments, the promoter-less donor vector selected from the group consisting of plasmid, viral vector, and bacterial artificial chromosome (BAC). In certain embodiments, the promoter-less donor vector comprises at least four polyadenylation signals upstream from the transgene or nucleic acid encoding the RNA. In certain embodiments, the promoter-less donor vector further comprises a post-transcriptional regulatory element. In certain embodiments, the promoter-less donor vector further comprises a polyadenylation signal downstream from the transgene or nucleic acid encoding an RNA. In certain embodiments, the promoter-less donor vector comprises: a PGK polyadenylation signal (pA); a trimerized SV40pA; the transgene or nucleic acid encoding an RNA; loxP and flippase recognition target (FRT); a rabbit beta-globin pA; and a woodchuck hepatitis virus post-transcriptional regulatory element (WPRE). In certain embodiments, the paired recombinase recognition sites are loxP and flippase recognition target (FRT), and the recombinases are cre and flp. In certain embodiments, the paired recombinase recognition sites are VloxP and flippase recognition target (FRT), and the recombinases are VCre and flp. In certain embodiments, the paired recombinase recognition sites are SloxP and flippase recognition target (FRT), and the recombinases are SCre and flp. In certain embodiments, the recombinase is PhiC31 recombinase and the recombinase recognition sites are attB and attP. In certain embodiments, the wherein the recombinase is Nigri, Panto, or Vika and recombinase recognition sites are nox, pox, and vox, respectively. In certain embodiments, wherein one or both of the paired recombinase recognition sites comprise a mutation. In certain embodiments, the RNA is siRNA, snRNA, sgRNA, lncRNA or miRNA. In certain embodiments, the transgene or the nucleic acid encoding an RNA comprises disease associated mutations. In certain embodiments, the transgene or the nucleic acid encoding an RNA comprise a gain-of-function (GOF) gene mutation, loss-of-function (LOF) gene mutation, or both. In certain embodiments, the transgene comprises a factor that prevents apoptosis or promotes survival of a neuronal cell, increases the proliferation of a neuronal cell, or promotes differentiation of a neuronal cell. In certain embodiments, the factor is a growth factor. In certain embodiments, the growth factor comprises glial cell line-derived neurotrophic factor (GDNF), neurturin, growth/differentiation factor (GDF) 5, mesencephalic astrocyte-derived neurotrophic factor (MANF), cerebral dopaminergic neurotrophic factor (CDNF), or combinations thereof. In certain embodiments, the growth factor comprises glial cell line-derived neurotrophic factor (GDNF). In certain embodiments, the donor vector comprises an open reading frame (ORF) that begins with a splice acceptor. In certain embodiments, the donor vector comprises a fluorescent reporter. In certain embodiments, provided herein, is a mammalian cell comprising the system. In certain embodiments, the cell is a human cell. In certain embodiments, the cell is a pluripotent cell. In certain embodiments, the pluripotent cell is an induced pluripotent cell. In certain embodiments, the cell is for use in a method of delivering a gene product (e.g., growth factor, neurotrophic factor) to a subject having a neruodegnerative disorder, the method comprising administering the mammalian cell to the individual. In certain embodiments, the neurodegenerative disorder comprises Parkinson's Disease, Amyotrophic Lateral Sclerosis (ALS), or Alzheimer's Disease. In certain embodiments, the neurodegenerative disorder comprises Parkinson's Disease. In certain embodiments, the neurodegenerative disorder comprises Amyotrophic Lateral Sclerosis (ALS). In certain embodiments, the cell is for use in a method of increasing GDNF protein level in the brain of in an individual, the method comprising administering the mammalian cell to the individual.

In another aspect, provided herein, is a promoter-less donor vector, comprising: a polyadenylation signal or transcription stop element upstream from a transgene or nucleic acid encoding an RNA; the transgene or nucleic acid encoding an RNA; and paired recombinase recognition sites. In certain embodiments, the promoter-less donor vector selected from the group consisting of plasmid, viral vector, and bacterial artificial chromosome (BAC). In certain embodiments, the promoter-less donor vector comprises at least four polyadenylation signals upstream from the transgene or nucleic acid encoding the RNA. In certain embodiments, the transgene or RNA is selected from the group consisting of an oncogene, loss-of-function (LOF) mutation of a tumor suppressor gene, gain-of-function (GOF) mutation of a proto-oncogene, pseudogene, siRNA, snRNA, sgRNA, lncRNA, miRNA, epigenetic modification, non-coding genetic or epigenetic abnormality associated with human disease, and combinations thereof. In certain embodiments, the promoter-less donor vector further comprises a post-transcriptional regulatory element. In certain embodiments, the promoter-less donor vector further comprises a polyadenylation signal downstream from the transgene or nucleic acid encoding an RNA. In certain embodiments, one or both of the paired recombinase recognition sites comprise a mutation. In certain embodiments, the promoter-less donor vector comprises: PGK polyadenylation signal (pA); trimerized SV40pA; a transgene or RNA; loxP and flippase recognition target (FRT); a rabbit beta-globin pA; and a woodchuck hepatitis virus post-transcriptional regulatory element (WPRE). In certain embodiments, the transgene comprises a factor that prevents apoptosis or promotes survival of a neuronal cell, increases the proliferation of a neuronal cell, or promotes differentiation of a neuronal cell. In certain embodiments, the factor is a growth factor. In certain embodiments, the growth factor comprises glial cell line-derived neurotrophic factor (GDNF), neurturin, growth/differentiation factor (GDF) 5, mesencephalic astrocyte-derived neurotrophic factor (MANF), cerebral dopaminergic neurotrophic factor (CDNF), or combinations thereof. In certain embodiments, the growth factor comprises glial cell line-derived neurotrophic factor (GDNF). In certain embodiments, provided herein, is a mammalian cell comprising the promoter-less donor vector. In certain embodiments, the mammalian cell is a human cell. In certain embodiments, the mammalian cell is a pluripotent cell. In certain embodiments, the pluripotent cell is an induced pluripotent cell. In certain embodiments, the cell is for use in a method of delivering a gene product (e.g., growth factor, neurotrophic factor) to a subject having a neruodegnerative disorder in an individual, the method comprising administering the mammalian cell to the individual. In certain embodiments, the neurodegenerative disorder comprises Parkinson's Disease, Amyotrophic Lateral Sclerosis (ALS), or Alzheimer's Disease. In certain embodiments, the neurodegenerative disorder comprises Parkinson's Disease. In certain embodiments, the neurodegenerative disorder comprises Amyotrophic Lateral Sclerosis (ALS). In certain embodiments, the cell is for use in a method of increasing GDNF protein level in the brain of in an individual, the method comprising administering the mammalian cell to the individual.

In another aspect, provided herein, is a method of genetic manipulation of a mammalian cell, comprising: transfecting or transducing the mammalian cell with the system described herein. In certain embodiments, the mammalian cell is a human cell, the system targets the AAVS1 locus, H11 locus, or HPRT1 locus, and the method is an in vitro or ex vivo method. In certain embodiments, the mammalian cell is a mouse cell, and the system targets the ROSA26 locus, Hipp11 locus, Tigre locus, ColA1 locus, or Hprt locus. In certain embodiments, the method further comprises administering to the cell or contacting the cell with one or more recombinase enzymes. In certain embodiments, the one or more recombinase enzymes comprise, a Cre recombinase, a flippase recombinase, a Cre and a flippase recombinase, a Nigri recombinase, a Panto recombinase or a Vika recombinase.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1, panels A-M, depicts MADR in mTmG mouse or human lines generates genetic reporter-defined populations in vitro
A) Flp-Cre vector catalyzes either Cre-mediated excision or dRMCE on Rosa26^{mTmG} allele in the presence a MADR donor vector, resulting in two distinct recombinant products.
B) Nucleofection of heterozygous Rosa26^{WT/mTmG} mNSCs result in three possible lineages: tdTomato+, EGFP+, and TagBFP2+.
C) Live imaging of representative cells with non-overlapping fluorescent colors. Scale bars, 100 μm
D) Schematic of cell preparation for single-cell western blot.
E) Frequency of fluorescence intensities comparing MADR and PiggyBac transgenic cells.
F) Representative examples of single-cell western blots for PiggyBac and MADR groups. (Note that this is not a pure population and so some cells express the Histone H3 loading control protein but no TagBFP2. Also, many lanes are empty as is typical for this assay.)
G) MADR-compatible TRE-SM-FP plasmids for MADR MAX.
H) Dox induces efficient SM-FP expression allowing for orthogonal imaging of 4 independent reporters in vitro. Scale bar, 100 μm
I) High magnification confocal z-section demonstrates that each cell expresses a single SM-FP reporter. Scale bar, 10 μm
J) Schematic of AAVS1 locus targeting for HUMAN MADR by TALEN or CRISPR/Cas9
K) HEK293T cells containing AAVS1-targeted MADR recipient site expressing tdTomato and TagBFP2-V5-nls Scale bar, 100 μm
L) MADR-HEK293T cells transfected with pDONOR SM-FP-myc (Bright) or TagBFP-3XFlag showing GFP or BFP autofluorescence among non-inserted tdTomato+ cells. Scale bar, 100 μm
M) High mag image of cells from L exhibiting tdTomato and SM-FP-myc in a mutually exclusive manner. Scale bar, 10 μm

FIG. 2, panels A-O, depicts MADR in heterozygous mTmG allows for efficient tracing of lineages in vivo
A) Standard postnatal electroporation protocol targeting the VZ/SVZ cells in P2 heterozygous Rosa26^{WT/mTmG} pups with DNA mixture of a Flp-Cre vector and a donor plasmid
B) Postnatal EP recapitulates in vitro nucleofection experiment and yields TagBFP2+ MADR along with EGFP+ and tdTomato+ lineages at 2 weeks post-EP. Scale bar, 100 μm
C) Different concentrations of recombinase and donor plasmids result in various efficiencies of both MADR and Cre-excision recombination reactions in vivo. All mixtures contained a nuclear TagBfp2 reporter plasmid. (See FIG. 9D for representative images from this quantitation.) Error bars indicate standard error of the mean (SEM).
D) Schematic of plasmid delivery for combinatorial MADR MAX “brainbow” like multiplex labeling
E) Low mag image of olfactory bulb displaying multiplex SM-FP-based MADR MAX EPed cells and immunostaining for the SM-FP-linked epitope tags. Scale bar, 100 μm
F) High mag image of cells from E exhibiting expression of a single SM-FP epitope tag per neuron. Scale bar, 10 μm
G) Schematic of expansion microscopy and brightfield image example
H) pDonor SM-FP-myc sh.Nf1 miR-E plasmid for simultaneous knockdown of Nf1 and SM-FP-myc labeling of transgenic cells
I) Image of EPed striatum showing two populations of reporter labeled cells—EGFP and SM-FP-myc (i.e., Nf1 knockdown cells).
J) Pre-expansion SM-FP-myc cell body
K) Post-expansion of cell in J
L) Post-expansion EGFP astrocyte displaying “super-resolution” detail.
M) Schematic of pDonor-TagBFP2-P2A-VCre and FlEx VCre reporter plasmids for MATR (mosaic analysis with tertiary recombinase)
N) EPed striatum with FlpO-2A-Cre, pDonor-TagBFP2, HypBase and FlEx VCre reporter. Scale bar, 50 μm
O) Striatum of littermate of mouse shown in N with FlpO-2A-Cre, pDonor-TagBFP2-2A-VCre, HypBase and FlEx VCre reporter exhibiting VCre-dependent FlEx reporter (SM-FP-myc). Scale bar, 50 μm

FIG. 3, panels A-M, depicts loss-of-function manipulations using MADR transgenesis
A) Donor construct for miR-E shRNAs against Nf1, Pten, and Trp53 tied to TagBFP2 reporter
B) Validation of knockdown efficacy of multi-miR-E function by qPCR.
C) 6-month-old mouse sagittal section showing a hyperplasia of TagBFP2+ cells but no tumor. Scale bar, 1 mm
D) Plasmid for MADR of a TagBFP2-V5 reporter protein and SpCas9
E) Sequencing of TdTomato−/EGFP− glioma cells exhibit InDels in Nf1 and Trp53. SEQ ID NO:3, SEQ ID NO:4, SEQ ID NO: 5, SEQ ID NO:6, top to bottom, respectively.
F) MADR insertion of TagBFP2-V5 reporter and Cas9 with co-EPed PCR-derived sgRNAs yields high grade glioma observable through labeling of 3 genetic reporter-defined populations in a coronal section of both hemispheres. Scale bar, 1000 μm
G) Glioma cells are largely Olig2+ with small pockets of significant heterogeneity (white arrow). Scale bar, 1000 μm
H) High magnification Olig2 and tdTomato image focusing on the region denoted by the white arrow in FIG. 3G.
   Scale bar, 100 μm
I) CD44 and tdTomato immunostaining in a roughly adjacent section and region from FIG. 3H demonstrating positivity for the CD44 mesenchymal tumor marker. Scale bar, 100 μm
J) Plasmid for MADR of an SM_FP-myc reporter protein and FNLS Cas9n base editor.
K) sgRNA-targeting sites (green letters) induce C->T base conversion (red lowercase ‘c’ are targeted) to produce premature stop codons in Nf1, Trp53, and Pten. SEQ ID NO:7, SEQ ID NO:8, SEQ ID NO: 9, top to bottom sequences comprising sgRNA targeting sites, respectively. SEQ ID NO:10, SEQ ID NO:11, SEQ ID NO:12, SEQ ID NO:13, SEQ ID NO:14, SEQ ID NO:15, top to bottom peptides, respectively.
L) MADR insertion of myc reporter and FNLS Cas9n with co-EPed PCR-derived sgRNAs yields observable expansion of OPC progenitors at two months post-EP through labeling of three genetic reporter-defined populations in a coronal section. Scale bar, 1000 μm
M) High magnification tdTomato (1), EGFP (2), and Myc tag (3) image showing myc+ populations. Scale bar, 100 μm

FIG. 4, panels A-L, depicts generation of somatic glioma using in vivo MADR with Hras^{G12V} indicates dosage effects of this oncogene and human oncofusion proteins generate ependymal tumors
A-B) Schematic for in utero EP of MADR into E14.5 RCE+/−dams
C) In utero EP in RCE mice with Hras^{G12V} oncogene produces mosaic patches of TagBFP+ astrocytes Rosa26^{HrasG12} but not evidence of invasive glioma
D) Schematic of possible outcomes after MADR in homozygous mt/mg recipient mice
E) P2 EP of homozygous mt/mg mice with TagBFP2-Hras^{G12V} oncogene
F) Postnatal EP in homozygous Rosa26^{mTmG} P2 pups with Hras^{G12V} oncogene produces two different tumor types (Blue-only Rosa26^{HrasG12V×2} and blue-and-green Rosa26^{HrasG12V×1}) Scale bars: 2 mm
G) Representative tumor formation in homozygous mTmG 3 months post-EP. Blue-only Rosa26^{HrasG12V×2} cells occupy a larger section of the tumor than blue-and-green Rosa26^{HrasG12V×1}, correlating with phosphor-Rb1 protein expression. Scale bars: 1 mm
H) Zoom-in images of regions 1 and 2 from G show phosphorylated-Rb1 expression correlates largely with blue-only cells. Scale bars: 50 μm
I) Plasmid schematics for expression of ependymoma-associated fusion proteins
J) Stitch of YAP1-MAML1D; p16/p19 Cas9 targeting induced ependymoma-like tumor.
K) Survival analysis of Ependymoma MADR model mice
L) Ependymoma-like tumor in a 3 month old C11orf95-RELA; p16/p19 Cas9-targeted mouse

FIG. 5, panels A-Q, depicts generation of MADR glioma models utilizing recurrent mutations observed in pediatric GBM yields phenotypes consistent with human subtypes
A) Schematic of donor plasmid for MADR with multiple recurrent pediatric glioma driver mutations
B) Schematic of the plasmid delivery and electrode sweep employed to target striatal and cortical germinal niches simultaneously
C) Zoomed view from B showing the respective cortical (magenta) and striatal (orange) germinal niches that are targeted
D) Representative tumor formation in heterozygous mTmG 100 days post-EP. Nuclear EGFP+ Rosa26^{H3f3a-K27M/Pdgfra/Trp53} cells form a large striatal tumor. Inset D-1 shows a lack of significant cortical infiltration.
E) A littermate Rosa26^{H3f3aG34R/Pdgfra/Trp53} exhibits a glial hyperplasia in the striatum and cortex but no tumor is evident.
F) K27M tumor at 120 days post-EP is predominantly sub-cortical.
G) Cortically-infiltrating G34R tumor at 120 days post-EP.
H-I) Confocal pathology of K27M tumor at low mag (H), and high mag (I).
J) Low mag pathology of G34R tumor.
K) Comparison of survival across H3.3. groups (WT—blue, K27M—green, and G34R—red) all containing Pdgfra D842V and Trp53 R270H.
L) Chart of the site of K27M versus G34R tumors. *—Because of the later onset of tumor growth in G34R groups and their inconsistent survival times, we were unable to collect 2 of 7 G34R samples before death to definitively ascertain initial tumor site.
M-N) Experimental schematic for co-electroporation of K27M and G34R plasmids
O-P) G34R and K27M immunostaining of co-EPed tumors in sequential sections. (SM_FP-myc shown in insets.)
Q) Quantification of normalized cell counts from tumor

FIG. 6, panels A-L, depicts single-cell RNA-sequencing-based analysis of MADR glioma models
A) Schematic of cell dissociation and scRNA-seq
B) UMAP depicting CCA alignment of 3 MADR mouse K27M scRNA-seq datasets from 3 distinct tumors, colored by cluster based on HVG programs P1-4 from (Filbin et al., 2018)
C) Heatmap depicting marker genes emerging from unbiased clustering of mouse K27M cells
D) Program and expression featureplots from CCA of mouse K27M tumors.
E) UMAP depicting CCA alignment of 6 human K27M datasets from 6 distinct tumors (Filbin et al., 2018), colored by cluster
F) Heatmap depicting markers genes emerging from unbiased clustering of human K27M cells
G) Program and expression featureplots from CCA of human K27M tumors.
H) UMAP depicting CCA alignment of 3 MADR mouse K27M datasets and 6 human K27M datasets (Filbin et al., 2018), colored by cluster
I) Program and expression featureplots from CCA of combined mouse and human K27M tumors.
J) UMAP depicting CCA alignment of 9 K27M datasets from the mouse and human brain colored by sample
K) Heatmap using gene list from (Filbin et al., 2018) demonstrates a high concordance of gene expression between murine and human K27M glioma cells.
L) scRNA-seq derived proliferation metrics are comparable across mouse and human sample

FIG. 7, panels A-N, depicts H3.3 K27M Transcriptional Network and snATAC-seq Analysis
A) Heatmap depicting marker genes emerging from SCENIC binary regulon-based clustering of human K27M cells
B) SCENIC heatmap from mouse K27M cells
C) Binarized t-SNEs depicting regulon expression for EZH2, E2F1, MYBL1, and BRCA1 from human K27M samples
D) Binarized t-SNEs depicting regulon expression for Ezh2, E2f1, Mybl1, and BRCA1 from mouse K27M samples
E-F) t-SNEs depicting mRNA expression featureplots for genes in C and D. Note the lack of cluster specificity com-pared with regulons in C and D.
G-H) t-SNE featureplots depicting cell type-specific upregulation NANOG, OCT4, SOX2, MYC target genes, and embryonic stem cell (ES)-associated gene sets and the underexpression of PRC2, SUZ12, EED, and H3K27-bound gene sets for human cells (G) and analogous genes/genesets in mouse (H).
I) Schematic of snATAC-seq sample preparation
J) tSNE of sc- and snATAC datasets from P50, K) E18) and L) K27M mouse brains
M) MSigDB terms from snATAC-seq K27M tumor cells
N) Genome browser alignments of snATAC-seq, scATAC-seq, and bulk ATAC-seq. *—Tumor MG is an overlaid (red/black) alignment of snATAC-seq microglial clusters captured with the K27M cells. NPC—postnatal neural precursor cells; K27M ATAC—bulk mouse K27M tumor cells.

FIG. 8, panels A-N, depicts the measurement of MADR efficiency in heterozygous mTmG mNSCs by FACS analysis, confirmation of correct protein translation at non-clonal population level, inducible MADR, and MADR “proxy” lines, Related to FIG. 1. Schematic of recombinase-expressing plasmids (and minicircle) employed in this study
A) FACS analysis indicates the approximate MADR efficiency in neural stem cells, and no obvious difference between Flp-2A-Cre and Flp-IRES-Cre in their catalytic efficiencies
B) Sorted cells express Hras^{G12V} but not tdTomato or EGFP. Scale bar, 50 μm
C) Western blot indicating normal transgene production from non-clonal aggregate cells and lack thereof in FACS-negative population. Removal of tdTomato expression is also observed.
D) Schematics of plasmids and alleles subject to PCR analysis at denoted sites.
E) PCR screening analysis reveals that rtTA-V10-AU1 cassette is correctly integrated downstream of CAG-promoter in cells that are resistant to puromycin treatment
F) Western blot analysis of the cell line from FIG. 2C showing the expression of rtTA-V10-AU1 and also EGFP upon doxycycline induction
G) MADR-compatible TRE-EGFP plasmid
H) Heterozygous mTmG mNSCs are nucleofected with plasmid in G treated with puromycin, and turned into a colorless population. Scale bars: 10 μm
I) Induction of EGFP expression in the cell line that constitutively express rtTA-V10-AU1 . Scale bars: 50 μm
J) TRE cell line with a bidirectional tet-response element that expresses EGFP and Dll1 upon doxycycline treatment
K) Immunofluorescence of cells without and with Dox demonstrates the relative lack of leakiness and homogenous expression level of EGFP and mDll1. Scale bars: 20 μm
L) qPCR measurement of mRNA abundance before and after Dox addition to medium. (Ctrl plasmid lacks mDll1 CDS but is otherwise identical to plasmid in K.)
M) mT/mG-based “Proxy” cell lines for testing MADR constructs in vitro. Mouse N2a cells underwent CRISPR/Cas9-dependent homology dependent repair (HDR) with the same plasmids used for engineering ROSA26 mT/mG. Subsequent MADR transduction and sorting was used to clone alternate reporter lines.
N) Mouse N2a cells were created with a stable insertion of CAG-LF-mTFP1 in the ROSA26 locus. FlpO-2A-Cre and pDonor mScarlet is used to demonstrate dRMCE of this line.

FIG. 9, panels A-N, depicts characterization of in vivo MADR and control experiments confirming specificity of integration, Related to FIG. 2
A) At 2 days post-EP, cells start expressing TagBFP2. Scale bars: 50 μm; Insets: 10 μm
B) Gliogenesis and radial glia 2 weeks post-EP. Arrow indicates rare green-and-blue double positive cells at the VZ. Neurons with both markers can be observed in the OB at this time point. Scale bars: 100 μm; Inset: 20 μm
C) Projection of confocal z stacks showing EGFP (mG) and TagBFP (MADR) cells 1 week post EP.
D) Foxj1 immunostaining of same region depicted in C. Note the localized nuclear label along the VZ region. *—vascular staining due to “mouse on mouse” immunostaining.
E) MADR TagBFP single positive radial glial cell, displaying no EGFP (mG).
F) Three MADR TagBFP and EGFP (mG) double-positive cells—all expressing the Foxj1 transcription factor. Note that there seems to be an inverse correlation of TagBFP and EGFP expression.
G) Magnification of the white box from F showing that the cells with the brightest MADR labeling has the dimmest EGFP.
H) High-magnification confocal image of a pair of TagBFP2+ satellite glia, which are negative for tdTomato and EGFP. Scale bars: 10 μm
I) Representative images of SM_FP-HA (donor), EGFP (mG), and TagBFP2-nls (blue) from VZ of the plasmid titration quantitations depicted in FIG. 2D.
J) Lineage tracing of EP-ed cells in the VZ/SVZ with hyPBase-integrated EGFP reporter plus various donor vectors and recombinases do not show any integration by 2 weeks post-EP. Scale bars: 100 μm
K) Donor vector with inverted loxP orientation fails to express Hras^{G12V} and does not produce hyperplasia. (For comparison of integrated plasmid at same time point, see FIG. 11A.) Scale bars: 100 μm. SEQ ID NO:16, SEQ ID NO:17, top and bottom, respectively.
L) Example of 5 color imaging for increasing spectral flexibility using Alexa 750 fluorophore.
M) Stitch of mT/mG brain immunostained with anti-EGFP in the 405 channel, anti-Olig2 in the 488 channel, and anti-Pdgfra in the 555 channel. H1) Note the intense tdTomato autofluorescence.
N) Stitch of same brain post-bleaching, showing significantly reduced mT tdTomato autofluorescence I1) Note the similar lack of detectable EGFP signal in the 488 wavelength due to bleaching.

FIG. 10, panels A-G, depicts the characterization of in vivo MADR loss of function lineages and comparison with CRISPR, Related to FIG. 3
A) At 3 months post-EP, cells expressing multi-miR-E tied to TagBFP2 reporter are predominantly Pdgfra+ OPCs. Scale bars: 100 μm
B) TagBFP2+ neurons in the olfactory bulb of multi-miR-E MADR mice.
C-D) Episomal Cas9-mediated multiplex mutation of Nf1, Trp53, and Pten yield transformation of piggyBac-transposed EGFP+ cells into Olig2+ tumors localized near white matter tracts.
E) V5⁺ tumor-derived cell populations can be found juxtaposed to the Tdtomato+ vasculature in focal regions of the tumor.
F) Confirmation of base editing to induce a premature stop codon in Pten using genomic alignment of sequenced amplicon.
G) MADR CRISPR/Cas9 variants generated for knockdown by Crispri (dCas9-KRAB-MeCP2) or Cas13/RX, or for knockout/genome editing with HiFi EspCas9. U6/miRFP670 reporters plasmids for expressing appropriate sgRNA variants have been constructed with sites for the BsmBI type II restriction enzyme for seamless sgRNA cloning and expression. CS—dual BsmBI cloning site

FIG. 11, panels A-L, depicts examination of MADR glioma and ependymoma cell fate changes and migratory dynamics, Related to FIG. 4
A) RCE-based Hras^{G12V} mosaic exhibiting Sox9/Gfap+ gliosis in TagBFP2+ regions.
B1 & B2) Mouse lines potentially compatible with in vivo MADR to allow lineage tracing studies or orthogonal RNA isolation using Ribotrap heterozygotes. Additionally, this method can extend to thousands of gene-trap mice that, as an example, flank loxP and FRT around important exons. in vivo MADR at such loci would enable i) lineage tracing of heterozygous/homozygous null cells at the locus, as well as ii) swapping the locus with a transgene. B1) SEQ ID NO:18—minimal FRT sequence, SEQ ID NO:19—FRT sequence, SEQ ID NO:18—minimal FRT sequence, SEQ ID NO:18—minimal FRT sequence, SEQ ID NO:18—minimal FRT sequence, top to bottom, respectively. B2) SEQ ID NO:18—minimal FRT sequence, SEQ ID NO:18 — minimal FRT sequence, top and bottom, respectively
C) Two weeks post-EP shows clear lineage divergence between EGFP+ cells that underwent Cre-mediated excision of tdTomato cassette and Hras^{G12V+} cells with successful MADR. Scale bars: 100 μm
D) As low as 10 ng/μl recombinase-expression vector in EP mixture can catalyze MADR in vivo. Scale bars: 100 μm
E) Brighter EGPF-Hras^{G12V} cells after pBase-mediated integration express phosphorylated Rb1. Scale bars: 200 μm
F-J) Striatal gliogenesis 1 month after electroporation of pDonor-(E) Kras G12A, (F-G) YAP1-MAML1D, or (H) C11orf95-RELA.
K-L) High magnification of ependymoma pushing margins displaying a lack of infiltration of these tumors.

FIG. 12, panels A-X depicts, characterization of multi-cistronic tumors, secondary elements, and viability screens, Related to FIG. 5
A) In vitro assessment of transgene expression after MADR in heterozygous mTmGmNSCs shows the co-expression of nuclear EGFP with Pdgfra, V5 (Trp53^{R270H}), and P53. Note the presence of contaminating mG cells with membrane EGFP and no tdTomato or transgene expression. Scale bars: 50 μm
B) Confirmation of Trp53 co-expression with nuclear EGFP (H3f3a). Scale bars: 50 μm
C) Coronal section displaying pre-tumor phase of K27M-expressing lineages (nuclear EGFP; and mG EGFP—membrane EGFP)
D-G) Immunostaining of K27M (D,G) and G34R (E-F) tumors with anti-H3mutK27M and anti-H3mutG34R antibodies, confirming expression of the respective transgenes by specific immunolabeling with the appropriate antibodies.
H) Dual immunostaining of K27M and G34R in co-electroporated animals (K27M- and G34R-containing plasmids) confirms expression of only one H3f3a mutant variant per cell.
I) Rosa26^{H3f3aG34R/Pdgfra/Trp53} EGFP+ tumor cells are hypomethylated at H3K27.
J) High mag view of tumor margin.
K) Immunolabeling of K27M mutant tumor cells demonstrates perinuclear satellitosis and decreased H3K27Me labeling compared with neighboring neurons.
L) CRISPR/Cas9 targeting of Nf1/Trp53 to induce GBM does not yield reduced H3K27Me3
M-N) H3K27Ac is observed in tumor cells but the intensity of labeling is not markedly increased compared with surrounding wildtype cells.
O-Q) Low mag (O-O2) and high mag images (P-Q) of Bmi1 upregulation in K27M tumor cells. Arrows point to infiltrating cells and dotted line depicts tumor margin. P) Max projection of region in O-O1 showing that infiltrating K27M cells are juxtaposed to vessels.
R) A subset K27M and G34R mutant cells at the margins can be immunolabeling with the astrocyte marker Aldh1l1 and display hypertrophy
S) Subpopulations of K27M and G34R mutant cells express the oligodendrocyte marker Cspg4.
T) Schematic of MADR plasmid for simultaneous generation of glioma and non-invasive imaging of tumor growth with Akaluc.
U) Control animal alongside littermate electroporated with plasmid from T and injected with akalumine.
V) MADR FUCCI variants, containing PIP degron fusions and hGEM1/110 fusions for discrimination of cell cycle events with different fluorescent proteins. Variants also have been generated for simultaneous generation of glioma and demarcation of cell cycle events with near infrared fluorescent proteins. Images show N2a proxy line with stable insertion of Venus/mCherry MADR FUCCI plasmid.
W) Schematic for derivation of tdTomato+ NPCs and EGFP+ tumor populations form the same microdissection for simultaneous “paired” toxicity screening.
X) Akt1/2 kinase inhibitor decreases proliferation in both NPCs and MADR K27M populations while Vacquinol-1 decreases proliferation preferentially in the K27M tumor population. Results are combined from 4 biological replicates and representative of two independent lines of each cell type.

FIG. 13, panels A-M, depicts single-cell RNA-seq of MADR mutant models, Related to FIG. 6
A) CNV analysis of 3 mouse K27M tumor scRNA-seq datasets
B) CC1 and CC2 vector alignment of mouse K27M tumors
C) Biweight midcorrelation plot of mouse K27M tumors across CCs
D) UMAP depicting CCA alignment of 3 K27M datasets from the mouse brain colored by sample
E) Featureplots depicting expression of genes in mouse K27M tumors.
F) Louvain clustering of human K27M scRNA-seq tumors from Filbin et al. (Science 2018).
G) CSF1R and H) MOG expression maps depicting clusters that are filtered before moving to CCA.
I) Clustering of Human K27M tumors post filtering.
J) Biweight midcorrelation plot of human K27M tumors across CCs
K) UMAP of CCA alignment human K27M tumors colored by sample.
L) Featureplots depicting expression of genes in human K27M tumors.
M) Program featureplots split from CCA of all samples (i.e., FIG. 6H) and depicted by original sample. Clustering by highly-variable genes rather than programs from Filbin et al. (Science 2018) leads to slightly altered clustering depending on the clustering parameters chosen.

FIG. 14, panels A-Z, depicts SCENIC, H3K27me3 ChIP-seq, and snATAC-seq analysis of MADR mutant models, Related to FIG. 7
(A,C,E,G,I) t-Distributed Stochastic Neighbor Embeddings (t-SNEs) of SCENIC processed K27M human tumor cells (B,D,F,H,J) SCENIC-derived t-SNEs of K27M mouse tumor cells. Samples are grouped by sample (A,B; i.e. patient or mouse of origin), cell type (C,D), S-phase score (E,F), G2M-phase score (G,H), and overlapping cell cycle phases (I,J).
K) General workflow for tumor dissociation and downstream analysis such as scRNA-seq, scATAC-seq, or ChIP-seq for H3K27Me3. Note same tumor source was used for scRNA-seq analysis and ChIP-seq but independent tumors were used for scATAC-seq samples.
L) Clustering of H3K27Me3 ChIP-seq data from 3 mouse K27M tumors
M) Scoring of scRNA-seq-derived UMAP for genes from clusters in L
N) tSNE of scATAC-seq dataset from P50 brain with numbered clusters
O) Marker genes for oligodendrocyte (Mog), OPC (Pdgfra), astrocyte (Aqp4), microglia (C1qb), neuron (Snap25), and interneuron (Gad2, Pvalb, Sst) populations. Note the distinct signal to noise for each cluster.
P) tSNE of 3 combined scATAC-seq datasets from E18 brain after standard clustering
Q) tSNE of samples from P post-Harmony alignment
R) tSNE of E18 datasets with clusters numbered
S) Gene accessibility for Sox9 (astrocytes/stem cells), Olig2 (stem cells/oligodendrocyte lineage), Csflr (microglia), and Gfap (astrocytes). Note the lack of clear population/cluster segregation for microglia or glial specificity of Sox9 and Olig2 compared with Gfap, which is more exclusive to discrete populations and readily associates with the glial clusters.
T) tSNE of scATAC-seq dataset derived from K27M tumor cells and co-capture innate immune populations
U) Gene accessibility for Sox9, Olig2, Csflr, and Gfap. Again, note the lack of clear population/cluster segregation of Sox9 and Olig2 compared with Gfap, which exhibits more accessibility. Csflr is noticeably more accessible than Sox9 and Olig2 and is associated with innate immune clusters.
V) CisTopic and CellRanger-based clustering of K27M tumor populations leads to subtly different subclustering of tumor and immune populations.
W) Gene accessibility clearly defines microglial populations but Sox9 and Sox10 fail to co-segregate in tumor, unlike in P50 normal brain.
X) K27M scRNA-seq featureplots blending Sox10 (green) and Sox9 (red) demonstrate that Sox10 and Sox9 are highly expressed throughout the tumor clusters and even within individual cells in agreement with gene accessibility in W and genome browser data (FIG. 7N)
Y) mSigDB terms for P50 astrocyte and OPC clusters
Z) Motifs enriched in DARs from K27M tumor clusters. Note the enrichment of IEGs and ES-associated TFs. 1. SEQ ID NO:20; 2. SEQ ID NO:21; 3. SEQ ID NO:22; 9. SEQ ID NO:23; 12. SEQ ID NO:24; 30. SEQ ID NO:25; 41. SEQ ID NO:26; 56. SEQ ID NO:27; 61. SEQ ID NO:28; 62. SEQ ID NO:29; 64. SEQ ID NO:30; 71. SEQ ID NO:31; 85. SEQ ID NO:32; 86. SEQ ID NO:33; 89. SEQ ID NO:34; 100. SEQ ID NO:35.

FIG. 15 depicts a schematic of conditions tested for MADR, SEMI-Lockin “loxP” MADR, and Locked in “loxP” MADR in two recipients HEK proxy cell lines and two pDonors mScarlet, and thus, four experimental conditions.

FIG. 16 depicts regular MADR and SEMI-Lock in “loxP” MADR-1 18 and 24 hours-post transfection on an IncuCyte time-lapse microscope (Note the increase of red fluorescent cells in RE-loxP mutant).

FIG. 17 depicts Lock in “loxP” MADR and SEMI-Lock in “loxP” MADR-2 18 and 24 hours-post transfection on an IncuCyte time-lapse microscope. (Note the increase of red fluorescent cells in RE-loxP mutant+LE-LoxP recipient condition)

FIG. 18 depicts the summary of results depicting the speed and efficiency of SEMI-Lock in MADR-1, Lock in MADR, MADR and SEMI-Lock in “loxP” MADR-2. (Note that both conditions with mutated donors exhibited better MADR insertion.)

FIG. 19 depicts the comparison of SEMI-Lock in MADR-1 and Lock in “loxP” MADR.

FIGS. 20A and 20B depicts SEMI-Lock in MADR-1, Lock in MADR in 18, 24, 30 and 36 hours post transfection, which display a remarkable increase in MADR efficiency compared to wild type LoxP sites.

FIG. 21 depicts QUASI Lock in MADR by binding properties.

FIG. 22 depicts the comparison of SIMI-Lock in “FRT” MADR-1 and Quasi-Lock in MADR.

FIG. 23 depicts SEMI-Lock in “FRT” MADR-1, Quasi-Lock in 12, 16, and 20 hours post transfection on an IncuCyte time-lapse microscope. Note the faster and the increase of MADR insertion with pDonors carrying RE-loxP mutant+LE-FRT mutant). Arrowheads depict red fluorescent cells.

FIG. 24 depicts representative viral MADR using AAV in vitro with MADR mT/mG recipient cell line and depicted plasmid elements. Two AAV viruses were used, one expresses FlpO-2A-Cre while the other has a non-expressed (inverted) TagBFP reporter gene. When the TagBFP is transduced into cells by itself, it doesn't appear to be expressed. However, in the presence of the FlpO-2A-Cre virus, cells with the MADR recipient locus appear to lose expression of the tdTomato and EGFP transgenes and begin to express TagBFP.

FIG. 25 depicts AAV pDonor CMV RevOrientation TagBFP2 3Flag+AAV FlpO Cre. 30 days post-transduction in mTmG mice (note the presence of many blue autofluorescent neuronal cell bodies only in this condition).

FIG. 26 depicts AAV pDonor CMV RevOrientation TagBFP2 3Flag negative control (note no tagBFP autofluorescence or Cre recombination [i.e. EGFP])

FIG. 27 depicts AAV FlpO Cre negative control (note extensive EGFP from Cre recombination but no TagBFP).

FIG. 28 shows the function MADR cassette, AAVS-pACT-loxP-TagBFP-V5-nls WPRE FRT, validated in human induced pluripotent stem cells.

FIG. 29 shows the tissue-specific action of MADR, GLAST-Flp-Cre and GFAP-Flp-CRE validated in vivo in mouse brain.

### DESCRIPTION OF THE INVENTION

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

In some embodiments, “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present, so long as the selected coding sequence is capable of being replicated, transcribed and translated in an appropriate host cell.

As used herein “paired” with respect to recombinase recognition sites refers to two recombinase recognition sites, one 5′ to a recited genetic element (e.g., gene of interest, promoter or other regulatory element) and one 3′ to the stated genetic element. Paired recombinase recognition sites may be identical (e.g., LoxP-LoxP), comprise a wild-type and a variant site (e.g., LoxP-Lox71 or the reverse), or sites of two different origins whether wild-type or variant (e.g., FRT-LoxP or FRT-Lox66). Wild-type LoxP comprises the sequence ATAACTTCGTATAATGTATGCTATACGAAGTTAT (SEQ ID NO:17). Wild-type FRT comprises the sequence GAAGTTCCTATTCTCTAGAAAGTATAGGAACTTC (SEQ ID NO:18). A variant of these sequences is any sequence that varies by one or more nucleotides and can be cleaved by its recombinase (e.g., Cre for Lox sites and Flippase for FRT sites). In certain embodiments, such variants may be cleaved by their recombinase at a lower efficiency.

In some embodiments, “promoter region” is used herein in its ordinary sense to refer to a nucleotide region including a DNA regulatory sequence, wherein the regulatory sequence is derived from a gene which is capable of binding RNA polymerase and initiating transcription of a downstream (3′-direction) coding sequence.

In some embodiments, “operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

In some embodiments, “promoter-less” as used herein with reference to a donor vector refers a vector that does not have a eukaryotic promoter.

Described herein are exogenous nucleic acids and vectors for use in rendering a cell transgenic. In certain embodiments, the cell is a mammalian cell. In certain embodiments, the mammalian cell is a human cell. In certain embodiments, the mammalian cell is a human cell with pluripotent capability such as a fetal cell, an embryonic stem cell, a precursor cell or an induced pluripotent cell. In certain embodiments, these transgenic cells are useful to deploy as a therapy for neurodegenerative disease.

In some embodiments, “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. In certain embodiments, the exogenous nucleic acids are targeted to a “safe” landing site. A “safe” site is a genomic region that is devoid of genes and their associated regulatory sequences, and possess a low likelihood of disrupting normal cellular function or initiating oncogenic transformation of a cell. In certain embodiments, the known safe site is the AAVS1 locus. Exogenous elements may be added to a nucleic acid construct, for example using genetic recombination. Genetic recombination is the breaking and rejoining of DNA strands to form new molecules of DNA encoding a novel set of genetic information.

As used herein, the terms “homologous,” “homology,” or “percent homology” when used herein to describe to a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

Also described herein are polypeptides encoded by the nucleic acids of the disclosure. The terms “polypeptide” and “protein” are used interchangeably to refer to a polymer of amino acid residues, and are not limited to a minimum length. Polypeptides, including antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein the terms “individual,” “subject,” and “patient” are interchangeable, and includes individuals diagnosed with, suspected of being afflicted with a neurodegenerative disease, or selected as having one or more risk-factors for a neurodegenerative disease. In certain embodiments, the individual is a mammal. In certain embodiments, the individual is a human person.

GEMM-based approaches still entail cumbersome mouse engineering and significant cross-breeding. Conversely, electroporation and viral transgenesis has enabled quick somatic transgenic investigations of development and disease but lack the precision of GEMMS. Transposons are becoming popular for producing stable somatic transgenics in developmental studies and in vivo tumor modeling. However, these methods suffer from random genomic insertions, position effect variation including transgene shutdown, and copy number variability. MADR overcomes the intrinsic disadvantages associated with these methods, and is a robust strategy for creating somatic mosaics with predefined insertion sites and copy numbers and requiring a negligible amount of colony maintenance. We demonstrated the versatility of MADR to generate combined modes (GOF/LOF) of mutations for multiple tumor drivers expeditiously and flexibly.

In one aspect, the methods herein utilize MADR to create mosaics and tumors in a host of tissues. Additionally, non-integrating viral vectors could be employed to deliver MADR constituents to avoid insertional mutagenesis. Provided in Table 1 is a comparison of in vivo genetic manipulation approaches. In some embodiments of a MADR method, the time for engineering is about 2 weeks per plasmid. In some embodiments of a MADR method, the copy number is 1-2 depending on zygosity of recipient. In some embodiments of a MADR method, breeding is performed with one line per target strain. In some embodiments of a MADR method, expression is generally stable depending on locus silencing. In some embodiments of a MADR method, payload is governed by plasmid limits. In some embodiments of a MADR method, focality depends on electrode orientation. In some embodiments of a MADR method, efficiency can be titered to approach 100% insertion. In some embodiments of a MADR method, transgenes can potentially hop in and out before Flp/Cre dilution. In some embodiments, a MADR method is compatible/complementary with other methods, e.g., orthogonal to CRISPR/Cas variants, HITI, Slendr, and/or Base writers.

TABLE 1
Comparison of approaches for in vivo genetic manipulation

| | Method | | | | | | | |
|---|---|---|---|---|---|---|---|---|
| | | Standard | Transposition- | | CRISPR | | | Base |
| | GEMM | EP | mediated EP | Virus | Cas9/Cpf1 | HITI | SLENDR | writing |
| Time for | Months | ~2 weeks | ~2 weeks | >4-6 | ~2 weeks | ~2 weeks | ~2 weeks | ~2 weeks |
| engineering | | per plasmid | per plasmid | weeks | per plasmid | (plasmid); | (plasmid); | per plasmid |
| and | | | | | | months | months | |
| generation | | | | | | (virus) | (virus) | |
| Copy | 1-2 per | Highly | Highly | Variable | 1-2 but not | 1-2 but not | 1-2 but not | 1-2 but not |
| number | knock-in | Variable | Variable | but likely | readily | readily | readily | readily |
| | | | (up to | less than | controllable | controllable | controllable | controllable |
| | | | hundreds) | EP | | | | |
| Breeding | More | Not | Not | Only | Not | Not | Not | Not |
| | complex | necessary | necessary | necessary | necessary | necessary | necessary | necessary |
| | for | | | for | | | | |
| | conditional | | | RCAS/Tva | | | | |
| | alleles | | | | | | | |
| Stability | Generally | Prone to | Prone to | Prone to | Expression | Expression | Expression | Expression |
| of | stable | dilution | silencing | silencing | dependent | dependent | dependent | dependent |
| Expression | depending | and/or | and | and | on mutation | on insertion | on insertion | on mutation |
| | on locus | silencing | insertional | insertional | site | site or | site or | site |
| | silencing | | effects | effects | | fusion | fusion | |
| | | | | | | partner | partner | |
| Payload | Limited | Typically | Typically | Limited | Typically | Typically | Typically | Typically |
| | by | governed | governed | to viral | governed | governed | governed | governed by |
| | targeting | by plasmid | by plasmid | payloads | by plasmid | by plasmid | by plasmid | plasmid |
| | construct* | limits* | limits* | | limits but | limits but | limits but | limits but |
| | | | | | viral variant | viral variant | viral variant | viral variant |
| | | | | | is subject | is subject | is subject | is subject |
| | | | | | to viral | to viral | to viral | to viral |
| | | | | | payloads* | payloads* | payloads* | payloads* |
| Focality | Depends | Focality | Focality | Diffusion | Focality | Focality | Focality | Focality |
| | on cis | depends on | depends on | pattern | depends on | depends on | depends on | depends on |
| | regulatory | electrode | electrode | unidirectional | electrode | electrode | electrode | electrode |
| | elements | orientation | orientation | from | orientation | orientation | orientation | orientation |
| | | | | injection | (plasmid | (plasmid | (plasmid | (plasmid |
| | | | | site | version) or | version) or | version) or | version) or |
| | | | | | viral spread | viral spread | viral spread | viral spread |
| | | | | | (AAV/LV) | (AAV) | (AAV) | (AAV/LV) |
| Efficiency | Typically | 100% | 100% | 100% | approaching | Typically | Typically | up to 80% |
| | 100% | | | | 100% but | <20% but | <5% | but off- |
| | | | | | off-targets | requires | | targets and |
| | | | | | and | minicircle | | heterogeneity |
| | | | | | heterogeneity | DNA | | unclear |
| | | | | | unclear; | production | | especially |
| | | | | | largely | to reach this | | when |
| | | | | | LOF | | | multiplexing |
| Other | Least | Plasmids | Random | Random | immunogenic, | Multiplexing | Multiplexing | immunogenicity |
| notes | amenable | rarely | insertions, | insertions, | hard to | mutant | mutant | unclear, |
| | to mixing | integrate | supraphysio- | potential | definitively | alleles | alleles | challenging |
| | and | or integrate | logical | supraphysio- | lineage | challenging | challenging | to |
| | matching | unpredictably | expression, | logical | trace, low | | | definitively |
| | mutations | | can be | expression, | HDR | | | lineage trace |
| | | | silenced, | can be | efficiency | | | mutant cells |
| | | | in and out | silenced, | | | | |
| | | | hopping of | can incite | | | | |
| | | | transgenes | cellular | | | | |
| | | | | immunity, | | | | |
| | | | | RCAS/Tva | | | | |
| | | | | models | | | | |
| | | | | often use | | | | |
| | | | | injection | | | | |
| | | | | of >50,000 | | | | |
| | | | | avian virus | | | | |
| | | | | producing | | | | |
| | | | | cells- | | | | |
| | | | | causing | | | | |
| | | | | potential | | | | |
| | | | | immune | | | | |
| | | | | interactions | | | | |
| | | | | and | | | | |
| | | | | trauma | | | | |

*BAC DNA can be utilized
**-this decreases total cell yields

The MADR method entails utilization of two different recombinases. One can restrict the cell type specificity of MADR targeting by carefully choosing the combinations of promoters driving the expression of recombinases. In some embodiments, in vivo MADR is performed with bacterial artificial chromosomes. A donor plasmid harboring large chunks of genomic fragments driving the expression of fluorescent reporter or recombinases, such as VCre, can be created with loxP and FRT sites added on each end, enabling further higher-complexity lineage tracing studies. In some embodiments, described herein is a self-excising FlpO-2A-Cre, which shifts the reaction equilibrium toward the complete integration. In some cases, this maximizes MADR efficiency.

Next generation sequencing has exponentially increased the catalogue of recurrent somatic mutations seen in tumors. Further, it is now increasingly appreciated that histologically similar tumors can often have disparate genetic underpinnings with different phenotypes (e.g. K27M vs. G34R). We show proof of principle for using MADR as a platform for rapid ‘personalized’ modeling of diverse glioma types by combining GOF and LOF mutations. To our knowledge, our MADR-based model is the only one successful at recapitulating the spatiotemporal regulation of tumor growth by K27M vs G34R mutations. Further, by unambiguously comparing K27M and G34R mutant cells side-by-side in vivo in individual animals—a unique advantage of MADR—we have observed the increased ability of K27M to accelerate tumor growth compared to G34R. Thus, while our K27M and G34R models are both 100% penetrant, these distinct mutations at closely situated residues exert distinct and powerful influences over tumor growth dynamics and tumor sites of origin. We noted a similarly remarkable pattern in our novel side-by-side comparisons of YAP1-MAMLD1 and C11orf95-RELA ependymoma models, whereby synchronized MADR transgenesis in the same cell populations led to disparate survival times. This suggests that the clinical age of onset for tumor subtypes may not by reflective only of cell origin or time of mutation, but also is highly-dependent on driver-mutation dictated growth dynamics. There is a “reverse chronology” in terms of enhancers that are activated after PRC2 complex inactivation. Using our novel models combined with single-cell approaches, our observations that K27M tumor cells exhibit a protracted pre-tumor stage culminating in a primitive ES-like transcriptional and epigenetic state is consistent with the possibility that K27M mutation exhibits this same reverse chronology reactivation of developmental enhancers.

In summary, our findings establish MADR as a robust genetic methodology, one which promises to democratize the generation of high-resolution GOF and LOF mosaics, allowing a small lab to model a wide spectrum of genetic subtypes in vivo. Additionally, this genetic framework is adaptable to the thousands of mouse lines already engineered with dual recombinase recognition sites, and can easily be adapted to any cell, organoid or organism that can be engineered with a MADR recipient site. Given MADR's ability to be combined with the existing arsenal of genetic approaches, its single-cell resolution, and its compatibility with sequencing technologies, these tools allow for efficient, higher throughput investigation of gene function in development and disease.

Accordingly, embodiments of the present invention are based, at least in part, from these findings.

Described herein is a system of nucleic acids and/or vectors for rendering a cell transgenic with a transgene of interest. The transgene can be flanked by two different recombinase recognition sites, such as LoxP and FLT, allowing for introduction of the transgene of interest into a specific site of the genome of a cell. In certain embodiments, the transgene of interest comprises a neurotrophic factor. In certain embodiments, the neurotrophic factor comprises glial cell line-derived neurotrophic factor (GDNF), neurturin, growth/differentiation factor (GDF) 5, mesencephalic astrocyte-derived neurotrophic factor (MANF), cerebral dopaminergic neurotrophic factor (CDNF), or combinations thereof. In certain embodiments, the neurotrophic factor comprises GDNF. In certain embodiments, two or more neurotrophic factors may be included on the same or different nucleic acids/vectors for targeting to the genome of a cell.

In certain embodiments, the transgene of interest is under the control of an inducible promoter. An inducible promoter allows transcription, and thus production, of a polypeptide encoded by the transgene of interest to be controlled by administration of an inducing agent. The inducible promoter is one that is not activated or only minimally activated in the absence of an inducing agent. This allows for the production of a neurotrophic factor to be tuned or adjusted in an individual that has been administered a vector that comprises the transgene or cells comprising a vector that comprises the transgene. This allows for enhanced safety and increased therapeutic potential, as levels of neurotrophic factor that are too high have unwanted side effects, and levels that are too low may not be therapeutically effective. In certain embodiments, the inducible promoter is a tetracycline-regulated promoter. In certain embodiments, the transgene of interest that is under the control of an inducible promoter comprises GDNF, neurturin, GDF 5, MANF, CDNF, or combinations thereof. In certain embodiments, the transgene of interest that is under the control of an inducible promoter is GDNF.

In certain embodiments, the systems, nucleic acids and/or vectors further comprise an expression cassette that constitutively expresses a synthetic transcription factor that is activated by a small-molecule compound. In certain embodiments, the, the synthetic inducible transcription factor is the reverse tetracycline-controlled transactivator (rtTA). The rtTA transactivator is inducible by a tetracycline class antibiotic such as doxycycline. In certain embodiments, the synthetic transcription factor is supplied on a second nucleic acid/vector or the same nucleic acid/vector as that of the neurotrophic factor under control of an inducible element.

In certain embodiments, the neurotrophic factor that can be supplied by the systems, vectors, and nucleic acids described herein comprises GDNF. A GDNF gene supplies, upon transcription and translation, a GDNF polypeptide to an individual that has been administered either the naked vector or a cell(s) comprising the vector. The GDNF gene is a nucleic acid sequence that encodes a GDNF polypeptide, and includes, for example, an open reading frame (ORF) lacking at least one or all introns from an endogenous GDNF gene. In certain embodiments, the GDNF gene is at least about 85%, 90%, 95%, 97%, 98%, 99%, or 100% homologous to the DNA sequence set forth in SEQ ID NO: 1. In certain embodiments, the GDNF gene encodes a polypeptide at least about 85%, 90%, 95%, 97%, 98%, 99%, or 100% identical to the amino acid sequence set forth in SEQ ID NO: 2.

In certain embodiments, the transgene can be flanked by insulator sequences. An insulator sequence is a genetic element that prevents propagation of heterochromatin, and can be used to “insulate” a transgene and its regulatory sequences form epigenetic silencing. In certain embodiments, the insulator sequence can be the gypsy insulator of Drosophila, a Fab family insulator, or the chicken β-globin insulator (cHS4).

The systems, nucleic acids and/or vectors described herein are useful in a method for the delivering a gene product to a subject having a neurodegenerative disease or condition. In certain embodiments, the nucleic acids and/or vectors are integrated at a known safe site in the genome in a cell to be administered to an individual with a neurodegenerative disease. The neurodegenerative disease can be Alzheimer's disease, Parkinson's disease, or Amyotrophic lateral sclerosis (ALS). Additionally, these nucleic acids and/or vectors are useful in a method to increase GDNF, neurturin, GDF 5, MANF or CDNF protein levels in the brain of an individual, the midbrain of an individual, or the substantia nigra of an individual. In certain embodiments, the nucleic acids/vectors are used in a method to increase GDNF protein levels in the brain of an individual, the midbrain of an individual, or the substantia nigra of an individual.

Methods of delivering a gene product to a subject having a neurodegenerative disease or condition are also described herein. In certain embodiments, the neurodegenerative disorder comprises Parkinson's Disease, Amyotrophic Lateral Sclerosis (ALS), or Alzheimer's Disease. In certain embodiments, the method comprises administering a cell comprising the nucleic acids/vectors described herein to an individual in need thereof. In certain embodiments, the method comprises administering a cell comprising the nucleic acids/vectors comprising an inducible GDNF described herein to an individual in need thereof.

Described herein, is a method for the delivering a gene product to a subject having a neurodegenerative disease or condition, or an individual afflicted with a neurodegenerative disease or condition, including administering a quantity of cells to the individual afflicted with the neurodegenerative disease or condition, wherein the cells comprise a genomic integrated vector comprising a GDNF gene operably coupled to an inducible promoter, and wherein the GDNF gene and the inducible promoter are flanked by non-viral tandem repeats or recombinase recognition sites.

Also described herein, is a method of increasing GDNF levels in the brain of an individual afflicted with a neurodegenerative disease or condition, including a) administering a quantity of cells to the individual afflicted with the neurodegenerative disease or condition, wherein the cells comprise a genomic integrated vector comprising a GDNF gene operably coupled to an inducible promoter, and wherein the GDNF gene and the inducible promoter are flanked by non-viral tandem repeats; and b) administering an inducing agent to the individual. In certain embodiments, the inducing agent is doxycycline.

Also described herein is a method of increasing GDNF levels in the brain of an individual afflicted with a neurodegenerative disease or condition, including administering an inducing agent to the individual; wherein the individual has previously been administered a quantity of cells, wherein the cells comprise a genomic integrated vector comprising a GDNF gene operably coupled to an inducible promoter activated by the inducing agent. In certain embodiments, the inducing agent is doxycycline.

### Systems

Various embodiments of the present invention provide for a system, comprising: a promoter-less donor vector, comprising a polyadenylation signal or transcription stop element upstream from a transgene or nucleic acid encoding an RNA, the transgene or nucleic acid encoding an RNA, and paired recombinase recognition sites; and one expression vector, comprising two genes encoding recombinases specific to the paired recombinase recognition sites. In certain embodiments, the promoter-less donor vector selected from the group consisting of plasmid, viral vector, and bacterial artificial chromosome (BAC).

Other embodiments of the present invention provide for a system, comprising: a promoter-less donor vector, comprising a polyadenylation signal or transcription stop element upstream from a transgene or nucleic acid encoding an RNA, the transgene or nucleic acid encoding an RNA, and paired recombinase recognition sites; and two expression vectors, the first expression vector comprising one gene encoding a first recombinase that is specific to one of the paired recombinase recognition sites, and the second expression vector comprising one gene encoding a second recombinase that is specific to the other of the paired recombinase recognition sites. In certain embodiments, the promoter-less donor vector selected from the group consisting of plasmid, viral vector, and bacterial artificial chromosome (BAC).

In various embodiments, the promoter-less donor vector comprises at least four polyadenylation signals upstream from the transgene or nucleic acid encoding the RNA. In various embodiments, the promoter-less donor vector comprises at 2, 3, 4, 5 or 6 polyadenylation signals upstream from the transgene or nucleic acid encoding the RNA.

In various embodiments, the promoter-less donor vector further comprises a post-transcriptional regulatory element. In various embodiments, the promoter-less donor vector further comprises a polyadenylation signal downstream from the transgene or nucleic acid encoding an RNA.

In various embodiments, the promoter-less donor vector further comprises an open reading frame (ORF) that begins with a splice acceptor.

In various embodiments, the promoter-less donor vector further comprises a fluorescent reporter.

In various embodiments, the viral vector is an adeno-associated viral (AAV) vector. In various embodiments, the AAV vector is AAV1, AAV2, AAV3, AAV4, AAV5, AAV6, AAV7, AAV8, or AAV9. In various embodiments the viral AAV vector is a hybrid AAV vector; for example, wherein the capsid is derived from another serotype displaying the cell tropism of choice.

In particular embodiments, the promoter-less donor vector comprises: PGK polyadenylation signal (pA); trimerized SV40pA; the transgene or nucleic acid encoding an RNA; loxP and flippase recognition target (FRT); a rabbit beta-globin pA; and a woodchuck hepatitis virus post-transcriptional regulatory element (WPRE).

As non-limiting examples, the paired recombinase recognition sites can be loxP and flippase recognition target (FRT), and the recombinases would be cre and flp; the paired recombinase recognition sites can be VloxP and flippase recognition target (FRT), and the would be are VCre and flp; the paired recombinase recognition sites can be SloxP and flippase recognition target (FRT), and the recombinases would be SCre and flp. As a further non-limiting example, the recombinase can be PhiC31 recombinase, and PhiC31 recognition sites can be attB and attP. PhiC31 recognizes the attB and attP sites and creates attR and attL sites. Thus, a plasmid with attB and a target site with attP will catalyze insertion in the presence of PhiC31. Also, as further non-limiting examples, the recombinases can be Nigri, Panto, or Vika and their cognate sites are nox, pox, and vox, respectively.

In various aspects the paired recombinase recognition sites are chosen to increase the efficiency of integration of transgene or inducible transgene into the genome of a host cell. In certain embodiments, a variant LoxP site is paired with a wild-type or variant FRT site. In certain embodiments, a variant FRT site is paired with a wild-type or variant LoxP site. In certain embodiments, a variant Lox selected from Lox71, Lox66, lox511, lox5171, lox2272 is paired with a wild-type or variant FRT site. In certain embodiments, a Lox71 site is paired with an FRT site or variant FRT site. In certain embodiments, a Lox66 site is paired with an FRT site or variant FRT site. In certain embodiments a variant FRT selected from FRT1, FRT2, FRT3, FRT4, FRT5, FRT12, FRT13, FRT14, FRT545 is paired with a wild-type FRT. In certain embodiments a variant FRT selected from FRT1, FRT2, FRT3, FRT4, FRT5, FRT12, FRT13, FRT14, FRT545 is paired with a wild-type LoxP. In certain embodiments, the choice of paired recombination sites increases the efficiency of transgenic insertion into a cellular genome by 25%, 50%, 75%, or 100% or more.

In various embodiments, one or both of the paired recombinase recognition sites comprise a mutation. In various embodiments, the mutation for loxP is selected from lox71, lox75, lox44, loxJT15, loxJT12, loxJT510, lox66, lox76, lox43, loxJTZ2, loxJTZ17, loxKR3, loxBait, lox5171, lox2272, lox2722, m2, and combinations thereof. In various embodiments, the mutation for FRT is selected from FRT+10, FRT+11, FRT−10, FRT−11, F3, F5, F13, F14, F15, F5T2, F545, f2161, f2151, f2262, f61, and combinations thereof.

The mutation can allow for better transgenesis, and thus, new transgenic mice do not need to be generated. Furthermore, combinatorial experiments can be applied in a shorter window of time which allows for results to be obtained immediately when more than two different donor plasmids are used. This is also valuable in models wherein the organisms develop faster than mice.

In various embodiments, the RNA in the system(s) is siRNA, snRNA, sgRNA, lncRNA or miRNA. In various embodiments, the transgene or the nucleic acid encoding an RNA comprises disease associated mutations. In various embodiments, the transgene or the RNA comprise a gain-of-function (GOF) gene mutation, loss-of-function (LOF) gene mutation, or both. In various embodiments, the transgene or RNA is selected from the group consisting of an oncogene, loss-of-function (LOF) mutation of a tumor suppressor gene, gain-of-function (GOF) mutation of a proto-oncogene, pseudogene, siRNA, snRNA, sgRNA, lncRNA, miRNA, epigenetic modification, non-coding genetic or epigenetic abnormality associated with human disease, and combinations thereof.

### Donor Vectors

Various embodiments of the present invention provide for a promoter-less donor vector, comprising: a polyadenylation signal or transcription stop element upstream from a transgene or nucleic acid encoding an RNA; the transgene or nucleic acid encoding an RNA; and paired recombinase recognition sites. In certain embodiments, the promoter-less donor vector selected from the group consisting of plasmid, viral vector, and bacterial artificial chromosome (BAC).

In various embodiments, the promoter-less donor vector comprises at least four polyadenylation signals upstream from the transgene or nucleic acid encoding the RNA. In various embodiments, the promoter-less donor vector comprises at 2, 3, 4, 5 or 6 polyadenylation signals upstream from the transgene or nucleic acid encoding the RNA.

In various embodiments, the promoter-less donor vector further comprises a post-transcriptional regulatory element. In various embodiments, the promoter-less donor vector further comprises a polyadenylation signal downstream from the transgene or nucleic acid encoding an RNA.

In various embodiments, the promoter-less donor vector further comprises an open reading frame (ORF) that begins with a splice acceptor.

In various embodiments, the promoter-less donor vector further comprises a fluorescent reporter.

In various embodiments, the viral vector is an adeno-associated viral (AAV) vector. In various embodiments, the AAV vector is AAV1, AAV2, AAV3, AAV4, AAVS, AAV6, AAV7, AAV8, or AAV9. In various embodiments the viral AAV vector is a hybrid AAV vector; for example, wherein the capsid is derived from the another serotype displaying the cell tropism of choice.

In particular embodiments, the promoter-less donor vector comprises: PGK polyadenylation signal (pA); trimerized SV40pA; the transgene or nucleic acid encoding an RNA; loxP and flippase recognition target (FRT); a rabbit beta-globin pA; and a woodchuck hepatitis virus post-transcriptional regulatory element (WPRE).

As non-limiting examples, the paired recombinase recognition sites can be loxP and flippase recognition target (FRT); the paired recombinase recognition sites can be VloxP and flippase recognition target (FRT); the paired recombinase recognition sites can be SloxP and flippase recognition target (FRT). As a further non-limiting example, the recombinase can be PhiC31 recombinase. PhiC31 recognizes the attB and attP sites and creates attR and attL sites. Also, as further non-limiting examples, the recombinases can be Nigri, Panto, or Vika.

In various aspects the paired recombinase recognition sites are chosen to increase the efficiency of integration of transgene or inducible transgene into the genome of a host cell. In certain embodiments, a variant LoxP site is paired with a wild-type or variant FRT site. In certain embodiments, a variant FRT site is paired with a wild-type or variant LoxP site. In certain embodiments, a variant Lox selected from Lox71, Lox66, lox511, lox5171, lox2272 is paired with a wild-type or variant FRT site. In certain embodiments, a Lox71 site is paired with an FRT site or variant FRT site. In certain embodiments, a Lox66 site is paired with an FRT site or variant FRT site. In certain embodiments a variant FRT selected from FRT1, FRT2, FRT3, FRT4, FRT5, FRT12, FRT13, FRT14, FRT545 is paired with a wild-type FRT. In certain embodiments a variant FRT selected from FRT1, FRT2, FRT3, FRT4, FRT5, FRT12, FRT13, FRT14, FRT545 is paired with a wild-type LoxP. In certain embodiments, the choice of paired recombination sites increases the efficiency of transgenic insertion into a cellular genome by 25%, 50%, 75%, or 100% or more.

In various embodiments, one or both of the paired recombinase recognition sites comprise a mutation. In various embodiments, the mutation for loxP is selected from lox71, lox75, lox44, loxJT15, loxJT12, loxJT510, lox66, lox76, lox43, loxJTZ2, loxJTZ17, loxKR3, loxBait, lox5171, lox2272, lox2722, m2, and combinations thereof. In various embodiments, the mutation for FRT is selected from FRT+10, FRT+11, FRT−10, FRT−11, F3, F5, F13, F14, F15, F5T2, F545, f2161, f2151, f2262, f61, and combinations thereof. The mutation can allow for better transgenesis, and thus, new transgenic mice do not need to be generated. Furthermore combinatorial experiments can be applied in a shorter window of time which allows for results to be obtained immediately when more than two different donor plasmids are used. This is also valuable in models wherein the organisms develop faster than mice.

In various embodiments, the RNA in the system(s) is siRNA, snRNA, sgRNA, lncRNA or miRNA. In various embodiments, the transgene or the nucleic acid encoding an RNA comprises disease associated mutations. In various embodiments, the transgene or the RNA comprise a gain-of-function (GOF) gene mutation, loss-of-function (LOF) gene mutation, or both. In various embodiments, the transgene or RNA is selected from the group consisting of an oncogene, loss-of-function (LOF) mutation of a tumor suppressor gene, gain-of-function (GOF) mutation of a proto-oncogene, pseudogene, siRNA, snRNA, sgRNA, lncRNA, miRNA, epigenetic modification, non-coding genetic or epigenetic abnormality associated with human disease, and combinations thereof.

In particular embodiments, the promoter-less donor vector comprises: PGK polyadenylation signal (pA); trimerized SV40pA; a transgene or nucleic acid encoding an RNA; loxP and flippase recognition target (FRT); a rabbit beta-globin pA; and a woodchuck hepatitis virus post-transcriptional regulatory element (WPRE).

### Methods

Various embodiments provide for a method of genetic manipulation of a mammalian cell, comprising: transfecting or transducing the mammalian cell with a system of the present invention.

In various embodiments, the mammalian cell is a human cell and the system of the present invention targets AAVS1 locus, H11, HPRT1, or ROSA26, and the method is an in vitro or ex vivo method.

In various embodiments, the mammalian cell is a mouse cell and the system of the present invention targets ROSA26, Hipp11, Tigre, ColA1, or Hprt. In these embodiments, the method is in vitro, in vivo, or ex vivo.

### Animal Models

Various embodiments of the present invention provide for a non-human animal model, comprising: a non-human animal comprising a system of the present invention, wherein the transgene or RNA is selected from the group consisting of an oncogene, loss-of-function (LOF) mutation of a tumor suppressor gene, gain-of-function (GOF) mutation of a proto-oncogene, pseudogene, siRNA, shRNA, sgRNA, lncRNA, miRNA, epigenetic modification, non-coding genetic or epigenetic abnormality associated with human disease, and combinations thereof.

Various embodiments of the present invention provide for a non-human animal model, comprising: a non-human animal wherein a system of the present invention has been administered to the non-human animal, and wherein the transgene or RNA is selected from the group consisting of an oncogene, loss-of-function (LOF) mutation of a tumor suppressor gene, gain-of-function (GOF) mutation of a proto-oncogene, pseudogene, siRNA, shRNA, sgRNA, lncRNA, miRNA, epigenetic modification, non-coding genetic or epigenetic abnormality associated with human disease, and combinations thereof.

In various embodiments, the non-human animal model is a personalized non-human animal model of a human subject's cancer and the transgene or RNA is based on the human subject's cancer. In various embodiments, the non-human animal model is a personalized non-human animal model of a human subject's disease or condition and the transgene or RNA is based on the human subject's disease or condition. “Based on” as used in reference to “based on” a human subject's disease, condition, or cancer refers to having the transgene or RNA model the genetic profile of the human subject's disease, condition or cancer. As a non-limiting example, a transgene based on a human subject's cancer can be gene that is a gain-of-function genetic mutation that is believed to be a cause of the human subject's cancer.

In various embodiments, the non-human animal model comprises a gain of function mutation (GOF), a loss of function mutation (LOF), or both.

### Methods of Generating a Non-Human Animal Model or Human Cells

Various embodiments provide for a method of generating the non-human animal model of the present invention, comprising: transfecting or transducing the non-human animal model with a system of the present invention, wherein the transgene or RNA is selected from the group consisting of an oncogene, loss-of-function (LOF) mutation of a tumor suppressor gene, gain-of-function (GOF) mutation of a proto-oncogene, pseudogene, siRNA, shRNA, sgRNA, lncRNA, miRNA, epigenetic modification, non-coding genetic or epigenetic abnormality associated with human disease, and combinations thereof.

The system of the present invention, is as described above and herein.

### Drug Screening and Assessment

Various embodiments of the present invention provide for a method of assessing the effects of a drug candidate, comprising: providing the non-human animal model of the present invention; administering the drug candidate to the non-human animal model; and assessing the effects of the drug candidate on the non-human animal model.

In various embodiments, the method further comprises identifying the drug candidate as beneficial when the drug candidate provides beneficial results. In various embodiments, the method further comprises identifying the drug candidate and non-beneficial when the drug candidate does not provide beneficial results.

### Mammalian Cells

Various embodiments of the present invention provide for a mammalian cell comprising a system of the present invention as described herein. Other embodiments provide for a mammalian cell comprising a promoter-less donor vector of the present invention as described herein.

In various embodiments, the mammalian cell is a human cell. In various embodiments, the mammalian is a pluripotent cell. In various embodiments, the pluripotent cell is an induced pluripotent cell.

Various embodiments of the present invention provide for a mammalian cell comprising a genomic integrated transgene, wherein the genomic integrated transgene comprises a neurotrophic factor, and is integrated at a genomic site comprising a AAVS1 locus, H11 locus, or HPRT1 locus.

In various embodiments, the mammalian cell is a human cell. In various embodiments, the human cell is an induced pluripotent stem cell.

In various embodiments, the neurotrophic factor comprises glial cell line-derived neurotrophic factor (GDNF), neurturin, growth/differentiation factor (GDF) 5, mesencephalic astrocyte-derived neurotrophic factor (MANF), cerebral dopaminergic neurotrophic factor (CDNF), or combinations thereof. In various embodiments, the neurotrophic factor is GDNF.

In various embodiments, the neurotrophic factor is under the control of an inducible promoter. In various embodiments, the inducible promoter is a tetracycline inducible promoter. In various embodiments, the neurotrophic factor and or the inducible promoter are flanked by one or more of a recombinase recognition site, a tandem repeat of a transposable element, or an insulator sequence.

### Methods of Use

Various embodiments of the present invention provide for a method of delivering a gene product to an individual with a neurodegenerative disease or disorder comprising administering a mammalian cell of the present invention as described herein.

In various embodiments, the neurodegenerative disease or disorder comprises Parkinson's Disease, Amyotrophic Lateral Sclerosis (ALS), or Alzheimer's Disease.

In various embodiments, the neurodegenerative disease or disorder comprises Parkinson's Disease.

In various embodiments, the neurodegenerative disease or disorder comprises Amyotrophic Lateral Sclerosis (ALS).

Various embodiments of the present invention provide for a method of increasing a GDNF protein level in the brain of in an individual comprising administering a mammalian cell of the present invention to the individual.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1—Experimental Procedures

All mice were used in accordance with the Cedars-Sinai Institutional Animal Care and Use Committee. Embryonic day (E) 0.5 was established as the day of vaginal plug. Wild-type CD1 mice were provided by Charles River Laboratories. Gt(ROSA)26Sortm4(ACTB-tdTomato,-EGFP)Luo/J and Gt(ROSA)26Sortm1.1(CAG-EGFP)Fsh/Mmjax mice (JAX Mice) were bred with wild-type CD1 mice (Charles River) or C57BL/6J mice to generate heterozygous mice. Male and female embryos between E12.5 and E15.5 were used for the in utero electroporations, and pups between postnatal day (P) 0 and P21 for the postnatal experiments. Pregnant dams were kept in single cages and pups were kept with their mothers until P21, in the institutional animal facility under standard 12: 12 h light/dark cycles.

### Plasmid Cloning

The pDonor plasmids were derived from PGKneotpAlox2, using In-Fusion cloning (Clontech) or NEBuilder HiFi DNA Assembly Master Mix (NEB) in combination with standard restriction digestion techniques (Breunig et al., 2015, Soriano, 1999). Briefly, FRT site was created by annealing two oligos and infusing the insert into PGKneot-pAlox2. Downstream generation of donor plasmids were done by removing the existing ORF and adding a new cassette using In-Fusion or ligation, as was done for the smFP-HA ORF (Addgene 59759). PB-CAG-plasmids were previously described and created using combination of In-Fusion, NEB assembly, and ligation strategies (Breunig et al., 2015, Breunig et al., 2012). Primer sequences used for In-Fusion or assembly reactions are avail-able upon request. PCR was done using a standard protocol with KAPA HiFi PCR reagents. The original CMV Flp-2A-Cre and CMV Flp-IRES-Cre recombinase expression constructs were previously validated in the context of in vitro dRMCE (Anderson et al., 2012).

### MADR+AAVS1 Human Cell Line Generation

AAVS1 targeting MADR vector was derived from AAVS1-targeting vector AAVS1_Puro_PGK1_3×FLAG_Twin_Strep (Addgene 68375). TagBFP2-V5-nls-P2A-puroR-Cag-LoxP-TdTomato-FRT was inserted into this AAVS1 vector, and a human cell line was transfected with it and selected in puromycin. MADR-SM_FP-myc (bright) and MADR-TagBFP2-3flag WPRE was transfected into the resulting stable cell line with Cag-Flpo-2A-Cre to induce the MADR reaction.

### PCR Analysis of MADR Integration Events

KAPA HiFi PCR reagents were used to PCR genomic DNA collected from mouse MADR lines. Amplicons were run on an E-Gel apparatus to assess size.

### Mice and Electroporation

Gt(ROSA)26Sortm4(ACTB-tdTomato,-EGFP)Luo/J and Gt(ROSA)26Sortm1.1(CAG-EGFP)Fsh/Mmjax mice (JAX Mice) were bred with wild-type CD1 (Charles River) or C57BL/6J (JAX) mice to generate heterozygous mice. Postnatal lateral ventricle EPs were performed as previously described (Breunig et al., 2015). P1-3 pups were placed on ice for ˜5 min. All DNA mixtures contained 0.5-1 μg/μl of Flp-Cre expression vector, donor plasmid, hypBase, or CAG-reporter plasmids diluted in Tris-EDTA buffer, unless noted otherwise. Fast green dye was added (10% v/v) to the mixture, which was injected into the lateral ventricle. Platinum Tweezertrodes delivered 5 pulses of 120 V (50 ms; separated by 950 ms) from the ECM 830 System (Harvard Apparatus). SignaGel was applied to increase conductance. Mice were warmed under a heat lamp and returned to their cages.

### In Utero Electroporation

In utero electroporation experiments were performed according to standard methods (McKenna et al., 2011). TagBFP2-HRasG12V and Flp-Cre plasmids were EPed into E14.5 RCE mice embryos. After electroporation, the embryos were allowed to survive to P15, at which time TagBFP2-HrasG12V (MADR mediated insertion), EGFP (non-MADR Cre-mediated recombination) and Sox2 expression was analyzed by immunostaining.

### Supplementary Note on MADR Transduction

In our experimentation, we have successfully employed in vivo electroporation, in vitro electroporation (i.e. nucleofection), and lipofection to effect MADR.

In vivo electroporation is believed to work by allowing plasmid DNA to permeate the plasma membrane and enter the nuclear space of cells undergoing mitosis. Thus, it is believed to be largely specific for the proliferating populations. However, postmitotic cells may be also targeted by mixing nuclear pore dilators with the DNA.

As we have shown in our description of MADR, this approach facilitates stable expression of single-copy transgenes for studying development and disease. The number of MADR transduced cells is largely dictated by the concentration of the MADR donor, the concentration of FlpO and Cre recombinases, and the proliferation rate of the targeted populations. Specifically, as we have shown, the number of MADR cells versus Cre recombined cells can be titrated in a defined population by varying the ratio of donor plasmid to recombinase plasmid.

However, as can be seen in our postnatal electroporations, we note that under the standard conditions that we have chosen (100 ng/ul of recombinase: 1000 ng/ul of donor plasmid), a pattern emerges whereby MADR transduction inversely correlates with the initial mitotic activity of the cells. Specifically, striatal glia are readily Cre recombined but are more rarely MADR transduced. Conversely, the radial glial populations, which are relatively more quiescent as bona fide neural stem cells, make up a major population of MADR cells. Notably, ependymal cells, which have been recently reported to be the result of terminal asymmetric or symmetric divisions tend to be readily targeted by MADR—presumably due to the fact that they don't dilute the plasmids after the initial cell division targeted by electroporation. The cell cycle of the CNS lengthens over development, and postnatal cells are relatively more quiescent than their embryonic counterparts so smaller initial populations are typically transduced by postnatal electroporation. Thus, if large numbers of parenchymal glia or embryonically-generated neurons are desired, in utero electroporation may be performed targeting the local region (i.e., FIG. 4A-C).

### Size Considerations

We have not observed significant differences in MADR efficiency based on donor plasmid size between the standard ranges of plasmid DNA (4 Kb up to 18 Kb). Empirically testing using time-lapse imaging of MADR donors into proxy cells in vitro at 3 days post lipofection is in agreement with in vivo observations (data not shown). Plasmid mixes were based on identical molar ratios of individual donor variants. However, altering signaling pathways involved in cell fate, survival, proliferation, etc. will likely lead to changes in overall MADR cell numbers compared with using only genetic reporters.

### Cis-Regulatory Elements

We typically employ the strong CAG promoter due to its presence in the mouse lines that we utilize. However, there are several ways of attenuating the strength of this promoter:
1) Any IKNM mouse allele can be targeted with MADR so the transgenes could be regulated by the endogenous cis-regulatory elements.
2) We have demonstrated two orthogonal means for secondary induction of transgenes (Vcre, and Tet-On)—one of which is reversible and can be modulated by dosage of the induction agent (Tet-On). Moreover, other technologies (e.g. dimerization domains and destabilization domains) could also be employed to vary transgene function or expression.
3) Changes in the non-coding portion of the transcripts can have significant effects on transgene expression, including but not limited to WPRE removal, stuffer sequences, and miR-recognition sequences. WPRE has a potent effect on transcript perdurance and protein expression so removal will decrease expression of transgenes upstream. Also, one can specifically increase the number of elements in cistrons to create longer transcripts, which often leads to decreased overall expression. Finally, endogenous (or exogenous) miR-recognition sites can be used to tune expression in precise cell types (endogenous) or miR-hairpins with cognate or slightly mismatched targeting sequences can attenuate expression.
4) As is shown with our Akaluc plasmid (FIG. 8), a secondary cistron with an attenuated promoter can be inserted with MADR.

### Injection Site Inflammation

1) The pulled glass capillary tube has a very minute diameter-much smaller than a 30 G syringe. We have performed serial sectioning of several animals and have been unable to identify any needle track. Also, there is rarely bleeding induced by the injection. Thus, postnatal electroporation is considered a minimally invasive technique and a robust means of in vivo gene transduction.
2) One obvious concern is a possible microglial or astroglial reaction to the exogenous DNA at the injection site. However, we have not observed any significant inflammation compared to the control brain hemisphere (uninjected) in the days post-EP in the sections from our needle track analysis (data not shown). However, going too deep with the needle can lead to hydraulic trauma from the plasmid mixture, which can denude the surrounding ventricular walls.
3) For tumor-modeling purposes, there is a lengthy pre-tumor process (often spanning a few months), which gives substantial time for any tissue-injury-related inflammatory process to recede. This is still arguably better than viral-induced tumors or transplants into immunodeficient mice.
4) In utero electroporation (i.e. FIG. 4A-C) can be used as an alternate MADR delivery approach to additionally mitigate such issues by facilitating delivery into ventricles with a larger relative size and into embryos with a more immature immune system.

### Tissue Preparation

After anesthesia, mouse brains were isolated and fixed in 4% paraformaldehyde on a rotator/shaker overnight at 4° C. Brains were embedded in 4% low-melting point agarose (Thermo Fisher) and sectioned at 70 μm on a vibratome (Leica).

### Immunohistochemistry

Immunohistochemistry (IHC) was performed using standard methodology as previously described (Breunig et al., 2015). Agarose sections were stored in Phosphate Buffered Saline (PBS) with 0.05% sodium azide until use. Details on the primary antibodies can be found in the Table 3. All primary antibodies were used in PBS-0.03% Triton with 5% normal donkey serum. All secondary antibodies (Jackson ImmunoResearch) were used at 1:1000. Care was taken when including fast green dye for ventricle targeting in shorter duration experiments. Though the dye rapidly diluted in longer survival experiments, it confounded early (0-2 day) single-copy reporter detection and was omitted in these cases because of fluorescence in the far red wavelengths.

### Immunohistochemistry with Bleaching

For pre-bleached immunohistochemistry, 70 μm tissue sections were dehydrated with increasing concentrations of methanol (20%, 40%, 60%, 80%, 100%) for 15 minutes each in water at RT, and then treated overnight with 5% H₂O₂ in 100% methanol at 4° C. Tissue was then rehydrated using methanol (100%, 80%, 60%, 40%, 20%), 15 minutes each in water, and then washed with PBS before proceeding with normal immunostaining.

### Cell Culture and Nucleofection

Three heterozygous P0 mTmG pup brains were dissociated to establish the mouse neural stem cell line used in the study. The cell line was maintained as previously described (Breunig et al., 2015). Cells were grown in media containing Neurobasal®-A Medium (Life Technologies 10888-022) supplemented with B-27 without vitamin A (Life Technologies 12587-010), GlutaMAX (Life Technologies 35050), Antibiotic-Antimycotic (Life Technologies 15240), human epidermal growth factor (hEGF) (Sigma E9644), heparin (Sigma H3393), and basic fibroblast growth factor (bFGF) (Millipore GF003). Mouse NSC nucleofection was performed using the Nucleofector 2b device and Mouse Neural Stem Cell Kit according to manufacturer's recommendations (Lonza AG). The nucleofection mixtures contained plasmids with equal concentrations of 10 ng/μl.

### Live Cell Imaging

N2A proxy cells expressing PIP-Venus/mCherry-hGEM1/110 were plated in a 96-well format and imaged with at 20× objective lens under phase, red and green fluorescence using an Incucyte S3 System (Essen Bioscience, Ann Arbor, Mich.). Images were collected every 30 min using Incucyte S3 Software.

In high-throughput drug testing experiments, 10.000 cell from the cell lines generated from tumor dissociation and non-tumor control cells were plated in 96 well plates. 24 hours after the seeding appropriate concentration of each drug (1 μM for Vacquinol-1(Sigma-Aldrich, SML1187) and 0.5 μM for AKT 1/2 kinase inhibitor (Sigma- Aldrich, A6730)) was added to the media and cells were imaged for 92 hours in phase contrast using Incucyte S3 System. Images were collected every 2 hours using Incucyte S3 Software. Cell proliferation images analysis was done with Incucyte S3 software and normalized results presented and analyzed with Graphpad Prism 7.

### Imaging and Processing

All fixed images were collected on a Nikon A1R inverted laser confocal microscope. The live image of mNSCs was obtained on an EVOS digital fluorescence inverted microscope. For whole brain images, the automated stitching function of Nikon Elements was used. ND2 files were then imported into ImageJ to create Z-projection images, which were subsequently edited in Adobe Photoshop CS6. In several rotated images (e.g. FIG. 3F), rotation led to colorless space in the empty area completely outside of the tissue section and black fill was added. Adobe Illustrator CS6 was used for the final figure production.

### Quantification of In Vivo MADR Efficiency

For each condition, two pups were EPed with pCAG-TagBFP2-nls, pDonor-smFP-HA, and Flp-2A-Cre. The brains were taken two days post-EP, and two non-adjacent sections from each brain were stained with HA-Tag antibody and EGFP. For each section, ˜25 BFP+ cells were randomly selected, among which HA+ and EGFP+ cells among BFP+ cells were counted. The proportions were averaged over four sections for each group.

### Flow Cytometry

Cells were collected as previously described (Breunig et al., 2015). Cells were briefly rinsed in PBS, removed by enzymatic dissociation using Accutase (Millipore), pelleted at 250 g for 3 min, and resuspended in the media. FACS was done on a Beckman Coulter MoFlo at the Cedars-Sinai Flow Cytometry Core.

### Western Blot

The cell pellets were resuspended in laemmli buffer and boiled for 5 min at 95° C. Protein concentrations were measured on a ThermoScientific NanoDrop 2000. After SDS-PAGE separation and transfer onto nitrocellulose membranes, proteins were detected using the antibodies listed in the Table 3, diluted in 5% milk in 0.1% PBS-Tween. All secondary antibodies (Li-cor IRDye®) were used at 1:15000. Proteins were visualized by infrared detection using the Li-Cor Odyssey® CLX Imaging System.

### Single-Cell Western Blot

mTmG mNSCs were nucleofected (Lonza VPG-1004) with 6 μg of either piggybac or MADR TagBFP plasmid and 6 mg of FlpO 2A Cre in a T75 flask. After 4 days, cells were sorted through FACS, and 100,000-200,000 BFP+ cells were seeded onto Milo scWestern chips (ProteinSimple C300). Each chip was stained for guinea pig mKate (Kerafast EMU108) at 1:20 in Cy3 and rabbit histone H3 (Cell Signaling 4499) at 1:20 in 647. Imaging was performed using the Innoscan 710 microarray scanner.

### Doxycycline and Puromycin Administration

Doxycycline (Clontech 631311) was added to culture media at the final concentration of 100 ng/ml. Puromycin (Clontech 631305) was used at 1 μg/ml.

### Multi-miR-E Knockdown Efficiency Quantification

We have previously used FlEx-based transgene expression, specifically Cre-mediated inversion and activation of EGFP cassette (FlEx-EGFP). To test our multi-miR-E targeting Nf1, Pten, and Trp53, we made a CAG-driven FlEx-based construct harboring the multiple miR-Es (FlEx-multi-miR-E). Postnatal mNSC line was established by dissociating CD1 pup brains, transfected with EGFP or FlEx-multi-miR-E and Cre-recombinase vector. Fluorescent cells were sorted and subjected to mRNA extraction and SYBR-based Fluidigm BioMark dynamic array using qPCR probes for Nf1, Pten, and Trp53.

### Tissue Clearing

For whole mount imaging, the iDisco tissue clearing method was used (Renier et at 2014). Fixed samples were gradually dehydrated in 20%, 40%, 60%, 80%, 100%, 100% methanol/H₂O, 1 hour each at RT, and then bleached overnight in 5% H₂O₂ in 100% methanol overnight at 4° C., followed by a gradual rehydration (80%, 60%, 40%, 20% methanol/H2O, then PBS with 0.2% Triton X-100, 1 hour each at RT). Samples were then incubated in PBS with 0.2% Triton X-100, 20% DMSO, and 0.3M glycine for 2 days at 37° C. to permeabilize tissue, and then incubated in PBS with 0.2% Triton X-100, 10% DMSO, and 6% normal donkey serum for 2 days at 37° C. to block the tissue for staining. Samples were then incubated with primary antibodies in PBS with 0.2% Triton and 10 μg/ml heparin (PTwH), at 37° C. for 5 days, followed by 5 washes of PTwH, 1 hour each at RT, plus 1 overnight wash at RT. Samples were then incubated in secondary antibodies in PTwH, at 37° C. for 5 days, followed by 5 washes of PTwH, 1 hour each at RT, plus 1 overnight wash at RT.

Following staining, samples were again dehydrated gradually in 20%, 40%, 60%, 80%, 100%, 100% methanol/H₂O, 1 hour each at RT, and then stored overnight in 100% methanol at 4° C. Samples were then incubated in a solution of 66% dichloromethane (DCM, Sigma 270997) in methanol for 3 hours at RT, followed by 2 washes with 100% DCM, 15 minutes each at RT, and then placed directly into dibenzyl ether (DBE, Sigma 108014) for clearing and imaging. Cleared samples were stored in DBE in glass containers at RT in the dark. Samples were imaged in DBE using a light sheet microscope (Ultramicroscope II, LaVision Biotec) equipped with an sCMOS camera (Andor NEO 5.5) and a 2×/0.5 objective lens with a 6 mm WD dipping cap.

Light sheet datasets were imported into Imaris 9.1 (Bitplane) for 3D visualization. To digitally remove artifacts and fluorescent debris, the surface tool was used to create surface renderings of unwanted fluorescence, and the ‘mask all’ function in the surface menu was used to create fluorescence channels with debris removed. To create a digital surface of the whole sample, the volume-rendering tool was set to ‘normal shading’ and the color was set to gray. Movies of 3-D datasets were generated using the ‘animation’ tool.

### Expansion Microscopy

Samples were generated for expansion microscopy following the Pro-ExM protocol (Tillberg et al. 2015). Briefly, 100 μm sections were stained for EGFP and HA-tag. Before expansion, samples were imaged in water using a confocal microscope (Nikon A1R) for pre-expansion imaging.

Samples were anchored in 0.1 mg/ml Acryloyl-X, SE ((6-((acryloyl)amino)hexanoic acid, succinimidyl ester; Thermo-Fisher) in PBS with 10% DMSO, overnight at RT. After washing with PBS (3×10 minutes), samples were incubated for 30 minutes at 4° C. in monomer solution (PBS, 2 M NaCl, 8.625% (w/w) sodium acrylate, 2.5% (w/w) acrylamide, 0.15% (w/w) N,N-methylenebisacrylamide), immediately after addition of 0.2% (w/w) tetra- methylethylenediamine (TEMED), 0.2% (w/w) ammonium persulfate (APS), and 0.1% (w/w) 4-hydroxy-2,2,6,6-tetramethylpiperidin-1-oxyl (4-hydroxy-TEMPO). Slices were then incubated for 2 hours at 37° C. for gelation. After incubation, samples were incubated overnight in a 6-well plate at RT with no shaking in a digestion solution containing Proteinase K (New England Biolabs) diluted to 8 units/ml in digestion buffer (50 mM Tris pH 8, 1 mM EDTA, 0.5% Triton X-100, 1 M NaCl). Following digestion, samples were washed with excess H₂O 4 times, 1 hour per wash at RT, and then stabilized in 2% low melting agarose in H₂O before imaging. Images were acquired using a confocal microscope (Nikon A1R) with a 40× long WD objective (Nikon CFI Apo 40xw NIR).

### Pathology

After bleaching, immunohistochemistry was performed to stain for EGFP in the 405 channel. After incubation in secondary antibody, sections were incubated in 50 μM Draq5 (Cell signaling 4084S) in PBS for 2 minutes at RT, followed by washes of PBS (3×5 minutes). Sections were then incubated in 2% w/w Eosin Y (Sigma E4009) in 80% ethanol for 2 minutes at RT, followed by washes with PBS (3×5 minutes). Finally, sections were incubated in another Draq5 solution (50 μM in PBS) for 3 minutes, before washing with PBS, mounting, and imaging.

### In Vivo dRMCE Efficiency Titration

For each condition, pups were EPed with pDonor-smFP-Myc and Flpo-2A-Cre. The brains were taken two days post-EP, and two non-adjacent sections from each brain were stained with Myc-Tag antibody and EGFP. For each section, cells were quantified for insertion (Myc expressed) and cre excision (only EGFP expressed) using Syglass VR with an Oculus Rift system. Quantifications were indicated as percentages of total cells counted per section. The proportions were averaged over two sections from different animals for each group. Fast green was omitted from these assays as the dye was found to fluoresce in the same wavelengths as Alexa647. Though the dye rapidly diluted in longer survival experiments, it confounded early (0-2 day) single-copy reporter detection.

### PCR-Generation of U6-sgRNA Fragments

Reverse scaffold and forward primers (IDT DNA) were combined in a PCR reaction and subsequent purification to make concentrated sgRNAs (Ran et al., 2013). 100 ng of each fragment was combined with plasmid DNA for EP. We used previously-validated target sites for tumor modeling (Xue et al., 2014, Heckl et al., 2014) (Table 3).

### Sequencing InDel Mutations in Murine Tumor Cells

A pure population of tumor cells was obtained by FACS and genomic DNA was isolated (Qiagen DNeasy). Using primers flanking the gRNA target site, we PCR amplified the regions expected to contain InDel mutations for Nf1, Trp53, and Pten. The PCR amplified fragments were topo cloned using the Thermo Fisher Zero Blunt TOPO kit and transformed into One Shot MAX Efficiency DH5-T1R cells.

### Confirmation of CRISPR Base Edits

For premature stop codon base conversions, EGFP+ cells were obtained by FACS, and genomic DNA was isolated (Qiagen DNeasy). Using primers flanking the sgRNA target site, we PCR-amplified the regions expected to contain base conversions for Nf1, Trp53, and Pten. The amplicons were normalized to 20 ng/ul and sent for sequencing to the AMPLICON-EZ service (Genewiz).

Fastq files for each gene-primer pair were aligned to a custom genome file containing that gene locus using STARlong, and bwa-mem with default parameters, which all gave similar results. The BAM files were up-loaded to IGV for visualization.

### Akaluc In Vivo Bioluminescence Imaging

Stock Akalumine-HCL resuspended in dH₂O at 10 mM and stored in −80. Aliquot diluted in dH₂0 to a final con-centration of 5 mM and a final quantity of 10 uL/g w/v mouse, and IP injected prior to imaging. Mice were anaesthetized with isofluorane according to IACUC protocol, and imaged using IVIS Ilumina XRMS at 1.5 FOV and 60 s exposure rate.

### Tissue Dissociation

Mice were euthanized in CO₂ chamber and brains were collected in PBS. Immediately, EGFP+ tissue was micro-dissected under a Revolve Hybrid Microscope (Echo Labs, San Diego, Calif.). If allowed by the size of the tumor, some remains of the brain with residual tumor tissue was fixed in 4% PFA for tissue analysis. Microdissected tissue was mechanically dissociated into <1 mm pieces and further digested with Collagenase IV (Worthington Biochemical, Lakewood, N.J.), and DNAse I (Worthington Biochemical, Lakewood, N.J.). The resultant single cell suspension was filtered through 40 mm cell strainer (Stellar Scientific, Baltimore, Md.) and erythrocytes were lysed with ACK lysis buffer (Thermo Fisher Scientific, Waltham, Mass.). Single cell suspensions were split into 3 parts: First, for scRNAseq or sc-ATACseq experiments, GFP+ cells from single cell samples were FACS sorted (into 1.5 ml tubes for 10× Chromium). A secondary fraction was used for in vitro cell line establishment. Specifically, cells were resuspended in Neurobasal media (Thermo Fisher Scientific, Waltham, Mass.) supplemented with penicillin-streptomycin-amphotericin (Thermo Fisher Scientific, Waltham, MA), B-27 supplement without Vitamin A (Thermo Fisher Scientific, Waltham, Mass.), Glutamax (Thermo Fisher Scientific, Waltham, Mass.), EGF (Shenandoah Biotechnology, Warwick, Pa.), FGF (Shenandoah Biotechnology, Warwick, Pa.), PDGF-AA (Shenandoah Biotechnology, Warwick, Pa.) and heparin (StemCell Technologies, Cambridge, Mass.); and cultured in a CELLstart CTS (Thermo Fisher Scientific, Waltham, Mass.) treated T25 Flask. Finally, the last third of the single cell suspensions were fixed in 80% methanol-PBS and stored at −80 C.

### ScRNA-seq Library Generation

Single-cell RNA-seq libraries were prepared per the Single Cell 3′ v2 Reagent Kits User Guide (10× Genomics, Pleasanton, Calif.). Cellular suspensions were loaded on a Chromium Controller instrument (10× Genomics) to generate single-cell Gel Bead-In-EMulsions (GEMs). GEM-reverse transcription (RT) was performed in a Veriti 96-well thermal cycler (Thermo Fisher Scientific, Waltham, Mass.). After RT, GEMs were harvested and the cDNAs were amplified, cleaned up with SPRIselect Reagent Kit (Beckman Coulter, Pasadena, Calif.). Indexed sequencing libraries were constructed using Chromium Single-Cell 3′ Library Kit for enzymatic fragmentation, end-repair, A-tailing, adapter ligation, ligation cleanup, sample index PCR, and PCR cleanup. The barcoded sequencing libraries were quantified by quantitative PCR using the KAPA Library Quantification Kit (KAPA Bio-systems, Wilmington, Mass.). Sequencing libraries were loaded on a NovaSeq 6000 (Illumina, San Diego, Calif.) with a custom sequencing setting (26 bp for Read 1 and 91 bp for Read 2).

### ScRNA-seq Read Alignment

The demultiplexed raw reads were aligned to the transcriptome using STAR (version 2.5.1) (Dobin et al., 2013) with default parameters, using a custom UCSC mouse reference with mm10 annotation, containing all protein coding and long non-coding RNA genes. Expression counts for each gene in all samples were collapsed and normalized to unique molecular identifier (UMI) counts using Cell Ranger software version 2.0.0 (10× Genomics). The result is a large digital expression matrix with cell barcodes as rows and gene identities as columns.

To obtain 2-D projections of the population's dynamics, principal component analysis (PCA) was firstly run on the normalized gene-barcode matrix of the top 5,000 most variable genes to reduce the number of dimensions using Seurat package version 2.1-3 (Butler et al, 2018) in R v3.4.2-4.

### Nuclei Isolation for sc-ATACseq

GFP+ FACS sorted cells were processed following manufacture instruction for sc-ATACseq (10× Genomics, Pleasanton, Calif.). Specifically, sorted cells were filtered through a 40 mm cell strainer, pelleted and resuspended in one volume of lysis buffer (Tris-HCl 10 mM, NaCl 10 mM, MgCl2 3 mM, Tween-20 0.1% (Bio-Rad, 1610781), Nonidet P40 substitute 0.1% (Sigma-Aldrich, 74385), digitonin 0.01% (Sigma-Aldrich, 300410) and BSA 1% in Nuclease-fre water), cells were incubated on ice until optimal cell lysis. Then, lysis buffer was blocked by adding 10 volumes of Wash buffer (Tris-HCl 10 mM, NaCl 10 mM, MgCl₂ 3 mM BSA 1%, Tween-20 0.1% in Nuclease-free water). Isolated nuclei were pelleted and resuspended in 1× nuclei buffer (10× Genomics, Pleasanton, Calif.), Finally, nuclei concentration was calculated with an hematocytometer and proceeded immediately with sc-ATACseq library construction protocol.

### scATAC-seq Library Construction

scATAC sequencing library was prepared on the 10× Genomics Chromium platform following the manufacturer's protocol (10× Genomics 1000110). The isolated nuclei suspension was diluted and then incubated with trans-position mix for a targeted nuclei recovery of 10,000 cells. GEMs were then captured on the Chromium Chip E (10× Genomics 1000082). Following GEM incubation, clean up was performed using Dynabeads MyOne Silane beads (10× Genomics 2000048) and SPRIselect reagent (Beckman Coulter B23318). Finally, the library was amplified for a total of 10 SI PCR cycles.

### Human Single-Cell RNA-seq Data Processing

Three public processed data (GSE70630, GSE89567, and GSE102130) were obtained from their respective GEO websites. GSE70630 and GSE89567 were back-converted to TPM values. GSE102130 was divided into K27M (GSE102130_K27M) and GBM (GSE102130_GBM) datasets (6 and 3 patients, respectively). To identify the non-malignant microglia and mOGs in the datasets, we used PCA-tSNE and Louvain clustering as implemented in Scanpy (Wolf et al., 2018). The clusters containing the markers of microglia (CSF1R, LAPTM5, CD74, TY-ROBP) or mOGs (MBP, MOG, PLP1), as double-checked by t-test and Wilcoxon, were removed. For each dataset, the number of malignant tumor cells matched closely with those determined by the original authors (GSE70630: 4044 vs 4050, GSE89567: 5157 vs 5097, GSE102130: 2270 vs 2259). GSE102130 GBM did not contain any microglia or mOGs. For processing in Seurat, GSE102130_K27M was divided into 6 samples. All datasets, including the MADR mouse datasets, were normalized to have the library size of 10e5. For the comparative analysis across the tumor types, we used the relative expression as defined by (Filbin et al., 2018) to make the heatmap in FIG. 6K.

### Mouse Single-Cell RNA-seq Data Processing

The three 10×UMI count matrices (mK27M1, mK27M2, mK27M3) were normalized to have the library size of 10e5 for each cell. Then, we clustered in the same way as the public dataset to distinguish microglia and mOGs in Scanpy (Wolf et al., 2018). Cells that had more than 10% mitochondrial reads, less than 1000 unique reads, or more than 5000 unique reads were filtered out in Seurat (2.3.3) (Butler et al., 2018). After filtering, there were 2761, 562, and 3469 cells in mK27M1, mK27M2, and mK27M3, respectively.

### Seurat Processing

P1-4 genes were obtained from (Filbin et al., 2018) and used as the highly variable genes argument (genes.use) to identify the common substructures in each human and mouse dataset. The cells were clustered using CCA-UMAP (RunMultiCCA and DimPlot with ‘umap’), and the cluster-specific marker genes were identified using the Seurat function “find_all_markers” with the default arguments. To merge the mouse and human CCA-UMAPs, the mouse gene names were converted to their orthologous human counterparts using Ensembl BioMart (www.ensembl.org/biomart). For module scoring, the functions CellCycleScoring and AddModuleScore were used. The four gene lists (OC, AC, OPC, and Cycle) correspond to P1-4 genes. DoHeatmap function with at most top 50 genes for each cluster was used to make the heatmaps.

### SCENIC on Mouse and Human Dataset

SCENIC (1.0.0-02) was run with all default settings as described in (Aibar et al., 2017). We used the two default databases for each species (500 bp-upstream and tss-centered-10 kb). The raw matrices with the library size of 10e5 for each cell and the metadata dataframe from Seurat processing were used as inputs for SCENIC. For the heatmap and tSNE plotting, we used the binary regulon output. The package component AUCell was used to select a threshold for each regulon and then score each regulon for their enrichment in each cell (Aibar et al., 2017). The scores were then binarized (on vs off), and the outputs clustered according to this binary activity matrix (Aibar et al., 2017).

### Mouse Single-Cell ATAC-seq Data Processing

CellRanger was used to identify and annotate open chromatin regions and perform aggregation of samples and initial clustering of cells and motif analysis. CellRanger outputs were used as inputs for cisTopic and SnapA-TAC and samples were processed according to recommended settings (Bravo Gonzalez-Blas et al., 2019, Fang et al., 2019) for annotating clusters, Topics, ontology, gene accessibility, and motifs. The Harmony package (Korsunsky et al., 2018) was used according default settings in conjunction with SnapATAC to align E18 datasets.

### ChIP-seq Preparation

We completed the H3K27me3 ChIP reactions using 30 μg of mouse pediatric brain tumor chromatin and 4 μg of antibody (Active Motif, cat #39155). The ChIP reactions also contained a drosophila chromatin spike in for the normalization of the sequencing data. We diluted a small fraction of the ChIP DNA and performed qPCR using positive control primer pairs that worked well in similar assays. For H3K27me3, the primer pair targeted to the promoter region of the active gene ACTB serves as a good negative control.

### Histological Analyses

Nikon Elements and ImageJ software was used to analyze images. All results are shown as mean±SEM, except when indicated otherwise. For statistical analyses, the following convention was used: *: p<0.05, **: p<0.01, ***: p<0.001. “Student's t-test” refers to the unpaired test.

### Transcriptomic Analyses

The three 10×UMI count matrices (mK27M1, mK27M2, mK27M3) were normalized to have the library size of 10e5 for each cell. Then, we clustered in the same way as the public dataset to distinguish microglia and mOGs in Scanpy. Cells that had more than 10% mitochondrial reads, less than 1000 unique reads, or more than 5000 unique reads were filtered out in Seurat (2.3.3). After filtering, there were 2761, 562, and 3469 cells in mK27M1, mK27M2, and mK27M3, respectively. After filtering, there were 2761, 562, and 3469 cells in mK27M1, mK27M2, and mK27M3, respectively.

### ChIP-seq Analysis

ChIP-seq reads were aligned to the mouse reference genome mm10 using bwa. BigWig tracks were generated for each sample.

H3K27me3 clustering was performed using ngs.plot (version 2.61) (Shen et at, 2014) for each sample with mm10 mouse genome build. The list of genes associated with 7 clusters were imported to Seurat, and the expression for each cluster of genes was calculated using Seurat AddModuleScore.

### Base Editor Genotyping

The cells expressing EDITOR were subject to PCR amplification (list primers). Fastq files for each gene-primer pair were aligned to a custom genome file containing that gene locus using STARlong and bwa-mem with de-fault parameters, both of which gave similar results. The BAM files were uploaded to IGV for visualization.

TABLE 3

| | | |
|---|---|---|
| REAGENT or RESOURCE | SOURCE | IDENTIFIER |

Antibodies

| | | |
|---|---|---|
| chicken anti-EGFP | Abcam | Cat# ab13970, RRID: AB_300798 |
| goat anti-V5 | Abcam | Cat# ab95038, |
| | | RRID: AB_10676056 |
| rabbit anti-Sox9 | Abcam | Cat# ab185230, |
| | | RRID: AB_2715497 |
| rabbit anti-ALDH1L1 | Abcam | Cat# ab56149, RRID: AB_879534 |
| human anti-C-Myc Epitope Tag | Absolute Antibody | Cat# Ab00100-10.0 |
| rabbit anti-H3.3S31ph | Active Motif | Cat# 39637 |
| chicken anti-C-Myc Epitope Tag | Aves | Cat# ET-MY100, |
| | | RRID AB_2313514 |
| rat anti-CD44 | BD Biosciences | Cat# 550538, RRID: AB_39373 |
| rat anti-PDGFRα | BD Pharmingen | Cat# 558774, RRID: AB_397117 |
| mouse anti-Foxj1 | Invitrogen | Cat# # 14-9965-82 RRID: |
| | | AB_1548835 |
| rabbit anti-AU1 Epitope Tag | Biolegend | Cat# 903101, RRID: AB_256502 |
| sheep anti-p53 | Calbiochem | Cat# PC35, RRID: AB_2240806 |
| rabbit anti-H3K27Me3 | Cell Signaling | Cat# 9733, RRID: AB_2616029 |
| sheep anti-V5 | LSBio | Cat# LS-C136566, RRID: |
| | | AB_10915392 |
| rat anti-GFAP | Invitrogen | Cat# 13-0300, RRID: AB_2532994 |
| rabbit anti-HA | Cell Signaling | Cat# 3724, RRID: AB_1549585 |
| rabbit anti-pRB1 | Cell Signaling | Cat# 8516S, RRID: AB_11178658 |
| rabbit anti-Sox2 | Cell Signaling | Cat# 3579, RRID: AB_2195767 |
| rabbit anti-Bmi1 | Cell Signaling | Cat# 6964P, RRID: AB_10839408 |
| rabbit anti-H3K27Ac | Cell Signaling | Cat# 8173P, RRID: AB_10949887 |
| mouse anti-TetR | Clontech | Cat# 631132 |
| rabbit anti-Dsred | Clontech | Cat# 632496, RRID: AB_10013483 |
| mouse anti-V5 | Invitrogen | Cat# R960-25, RRID: AB_2556564 |
| rabbit anti-mCherry | Kerafast | Cat# EMU-106 |
| guinea pig anti-mKate2 | Kerafast | Cat# EMU108 |
| rat anti-Tdtomato | Kerafast | Cat# EST203, RRID: AB_2732803 |
| rabbit anti-H3F3A | Lifespan | Cat# LS-C148509-100, |
| | Biosciences | RRID: AB_11135921 |
| rabbit anti-H3F3A K27M | Millipore | Cat# ABE419, RRID: AB_2728728 |
| rabbit anti-NG2 | Millipore | Cat# AB5320, |
| | | RRID: AB_11213678 |
| sheep anti-Dll1 | R&D Systems | Cat# AF5026, RRID: AB_2092830 |
| goat anti-Olig2 | R&D Systems | Cat# AF2418, RRID: AB_2157554 |
| rabbit anti-H3.3G34R | Revmab | Cat# 31-1120-00, |
| | | RRID: AB_2716433 |
| rabbit anti-Atrx | Sigma | Cat# HPA001906, |
| | | RRID: AB_1078249 |
| mouse anti-Flag | Sigma Aldrich | Cat# F1804, RRID: AB_262044 |
| guinea pig anti-GFAP | Synaptic Systems | Cat# 173 004, RRID: AB_10641162 |

Bacterial and Virus Strains

| | | |
|---|---|---|
| One Shot MAX Efficiency DH5-T1R | Invitrogen | Cat# 12297016 |
| cells | | |
| Stellar chemically competent cells for | Clontech | Cat# 636766 |
| cloning | | |

Chemicals, Peptides, and Recombinant Proteins

| | | |
|---|---|---|
| Tris-EDTA buffer | Sigma-Aldrich | Cat# E8008-100ML |
| Fast Green Dye | Sigma Aldrich | Cat# F7258-25g |
| SignaGel Electrode Gel | Medline Industries | Cat# PLI1525CSZ |
| Low-Melting Point Agarose | Fisher Bioreagents | Cat# bp1360-100 |
| Human Epidermal Growth Factor | Sigma-Aldrich | Cat# E9644 |
| Heparin | Sigma-Aldrich | Cat# H3393 |
| Basic Fibroblast Growth Factor (bFGF) | Millipore | Cat# GF003 |
| Doxycycline | Clontech | Cat# 631311 |
| Puromycin | Clontech | Cat# 631305 |
| Methanol | Sigma Aldrich | Cat# 179337 |
| Hydrogen peroxide solution | Sigma Aldrich | Cat# H1009 |
| Triton X-100 | Sigma Aldrich | Cat# X-100-500ML |
| Dimethyl sulfoxide (DMSO) | Sigma Aldrich | Cat# D2650-5X10ML |
| Glycine | Sigma Aldrich | Cat# 410225-50g |
| Normal Donkey Serum | Jackson | Cat# 017-000-121 |
| | ImmunoResearch | |
| Dichloromethane | Sigma Aldrich | Cat# 270997 |
| Dibenzyl Ether | Sigma Aldrich | Cat# 108014 |
| Acryloyl-X, SE, 6- | Thermo-Fisher | Cat# A20770 |
| ((acryloyl)amino)hexanoic | | |
| Acid, Succinimidyl Ester | | |
| NaCl | Sigma-Aldrich | Cat# S9888 |
| Sodium Acrylate | Sigma-Aldrich | Cat# 408220 |
| Tetramethylethylenediamine | Sigma-Aldrich | Cat# T9281 |
| Ammonium Persulfate | Sigma-Aldrich | Cat# A3678-25g |
| 4-hydroxy-2,2,6,6-tetramethylpiperidin- | EMD Millipore | Cat# 840130 |
| 1-oxyl | | |
| Proteinase K | New England | Cat# P8107S |
| | Biolabs | |
| Draq5 | Cell Signaling | Cat# 4084S |
| Eosin Y | Sigma-Aldrich | Cat# E4009 |
| Collagenase IV | Worthington | Cat# LS004189 |
| | Biochemical | |
| DNAse I | Worthington | Cat# LS002007 |
| | Biochemical | |
| ACK Lysis Buffer | Thermo Fisher | Cat# A1049201 |
| | Scientific | |
| Neurobasal media | Thermo Fisher | Cat# 21103049 |
| | Scientific | |
| Penicillin-Streptomycin-Amphotericin | Thermo Fisher | Cat# 15240096 |
| | Scientific | |
| B-27 supplement without | Thermo Fisher | Cat# A3353501 |
| Vitamin A | Scientific | |
| Glutamax | Thermo Fisher | Cat# 35050061 |
| | Scientific | |
| Human EGF | Shenandoah | Cat# 100-26-500ug |
| | Biotechnology | |
| Human FGF (Shenandoah | Shenandoah | Cat# 100-146-100ug |
| Biotechnology, Warwick, PA), | Biotechnology | |
| PDGF-AA (Shenandoah | Shenandoah | Cat# 100-16-100 ug |
| Biotechnology, Warwick, PA) | Biotechnology | |
| Heparin Solution 0.2% | StemCell | Cat# 07980 |
| | Technologies | |
| CELLstart | Thermo Fisher | Cat# A10142-01 |
| | Scientific | |
| Akalumine-HCL | Sigma Aldrich | Cat#: 808350 |

Commercial Assays

| | | |
|---|---|---|
| DNeasy | Qiagen | Cat# 69504 |
| Zero Blunt TOPO kit | Thermo Fisher | Cat# 450159 |
| Chromium ™ Single Cell 3′ Library & | 10X Genomics | Cat# 120237 |
| Gel Bead Kit v2 | | |
| SPRIselect Reagent Kit | Beckman Coulter | Cat# B23318 |
| Chromium Single-Cell 3′ Library Kit | 10X Genomics | Cat# PN-120237 |
| KAPA Library Quantification Kit | Roche | Cat# 07960140001 |
| KAPA HiFi PCR kit | Kapabiosystems | Cat# KR0368 |
| In-Fusion cloning | Clontech | Cat# 638920 |
| Vacquinol-1 | Sigma-Aldrich | SML1187 |
| AKT 1/2 kinase inhibitor | Sigma-Aldrich | A6730 |
| Tween20 | Bio-Rad | 1610781 |
| digitonin | Sigma-Aldrich | 300410 |
| Nonidet P40 substitute | Sigma-Aldrich | 74385 |
| NEBuilder HiFi DNA Assembly | New England | Cat# E2621L |
| Master Mix | Biolabs | |
| Deposited Data | | |
| Mice raw and analyzed data | Herein | GEO: GSE117154, GSE131675, |
| | | GSE131672 |
| Human data | GEO website | GEO: GSE70630, GSE89567, |
| | | GSE102130 |
| P50 and E18 mouse scATAC data | 10X Genomics | www.10xgenomics.com/resources/ |
| | | datasets/ |
| Experimental Models: Cell Lines | | |
| Mouse MADR cell line: K27M-1 | Herein | N/A |
| Mouse MADR cell line: K27M-2 | Herein | N/A |
| Mouse MADR cell line: K27M-3 | Herein | N/A |
| Human: HEK293T | ATTC | Cat# CRL-3216 |
| Experimental Models: Organisms/Strains | | |
| Mouse: CD1 | Charles River | Strain Code 022 |
| | Laboratories | |
| Mouse: C57BL/6J | The Jackson | JAX: 000664 |
| | Laboratory | |
| Mouse: Gt(ROSA)26Sortm4(ACTB- | The Jackson | JAX: 007676 |
| tdTomato, -EGFP)Luo/J | Laboratory | |
| Mouse: Gt(ROSA)26Sortm1.1(CAG- | The Jackson | JAX: 32037 |
| EGFP)FshMmjax | Laboratory | |
| Oligonucleotides | | |
| sgRNA targeting sequence: Pten: | Herein | SEQ ID NO: 36 |
| gcCTCAGCCATTGCCTGTGTG | | |
| sgRNA targeting sequence: Trp53: | Herein | SEQ ID NO: 37 |
| GCCTCGAGCTCCCTCTGAGCC | | |
| sgRNA targeting sequence: Nf1: | Herein | SEQ ID NO: 38 |
| GCAGATGAGCCGCCACATCGA | | |
| sgRNA targeting sequence (BE): Pten: | Herein | SEQ ID NO: 39 |
| CCTcAGCCATTGCCTGTGTG | | |
| sgRNA targeting sequence (BE): | Herein | SEQ ID NO: 40 |
| Trp53: | | |
| CTGAGCcAGGAGACATTTTC | | |
| sgRNA targeting sequence (BE): Nf1: | Herein | SEQ ID NO: 41 |
| TCCTcAGTCACACATGCCAG | | |
| Recombinant DNA | | |
| plasmid: pDonor-TagBFP2-3XFlag | Herein | N/A |
| (cyto) WPRE | | |
| plasmid: pCag TagBFP2-V5 Cyto PB | Herein | N/A |
| plasmid: pDonor rtTA-V10-AU1-P2a- | Herein | N/A |
| puro-WPRE TRE-SM_FP-HA | | |
| plasmid: pDonor rtTA-V10-AU1-P2a- | Herein | N/A |
| puro-WPRE TRE-SM_FP-Myc | | |
| plasmid: pDonor rtTA-V10-AU1-P2a- | Herein | N/A |
| puro-WPRE TRE-SM_FP-Flag | | |
| plasmid: pDonor rtTA-V10-AU1-P2a- | Herein | N/A |
| puro-WPRE TRE SM_TagBFP-V5 | | |
| (weakly-fluorescent) | | |
| plasmid: pCag-FlpO-2A-Cre | Herein | N/A |
| plasmid: pGLAST-FlpO-2A-Cre | Herein | N/A |
| plasmid: pGFAP-FlpO-2A-Cre | Herein | N/A |
| plasmid: pCag-F5-FlpE-2A-Cre-F5 | Herein | N/A |
| plasmid: CMV FlpO-2a-Cre | Herein | N/A |
| plasmid: pAAV-Ef1a-flpo-2a-cre-wpre | Herein | N/A |
| plasmid: pAAV-(inverted; | Herein | N/A |
| promoterless) | | |
| TagBFP2-3Flag cyto-wpre | | |
| plasmid: CMV Flp-Ires-Cre | Herein | N/A |
| plasmid: AAVS1_Tagbfp2-V5-nls- | Herein | N/A |
| P2A-Puro_Cag LoxP myrTdtomato | | |
| FRT | | |
| plasmid: AAVS1_Bactin-loxP- | Herein | N/A |
| Tagbfp2-V5-nls- FRT | | |
| plasmid: AAVS1_Bactin-lox71- | Herein | N/A |
| Tagbfp2-V5-nls- FRT | | |
| plasmid: pDonor-SM_FP-myc (bright) | Herein | N/A |
| WPRE | | |
| plasmid: pDonor-SM_FP-myc (bright) | Herein | N/A |
| WPRE | | |
| plasmid: pDonor-SM_FP-Flag (bright) | Herein | N/A |
| WPRE | | |
| plasmid: pDonor-SM_FP-Myc (dark) | Herein | N/A |
| WPRE | | |
| plasmid: pDonor-SM_FP-HA (dark) | Herein | N/A |
| WPRE | | |
| plasmid: pDonor-SM_FP-flag (dark) | Herein | N/A |
| WPRE | | |
| plasmid: pDonor-mScarlet-3XSpot | Herein | N/A |
| WPRE | | |
| plasmid: pDonor-lox66-mScarlet- | Herein | N/A |
| 3XSpot WPRE-FRT | | |
| plasmid: pDonor-mScarlet-3XSpot | Herein | N/A |
| WPRE-FRT-10 | | |
| plasmid: pDonor-lox66-mScarlet- | Herein | N/A |
| 3XSpot WPRE-FRT-10 | | |
| plasmid: pDonor-mScarlet-3XSpot | Herein | N/A |
| WPRE-FRT-11 | | |
| plasmid: pDonor-lox66-mScarlet- | Herein | N/A |
| 3XSpot WPRE-FRT-11 | | |
| plasmid: pDonor-EGFP WPRE | Herein | N/A |
| plasmid: pDonor-lox66 EGFP WPRE- | Herein | N/A |
| FRT | | |
| plasmid: pDonor- EGFP WPRE-FRT- | Herein | N/A |
| 10 | | |
| plasmid: pDonor-lox66- EGFP WPRE- | Herein | N/A |
| FRT-10 | | |
| plasmid: pDonor- EGFP WPRE-FRT- | Herein | N/A |
| 11 | | |
| plasmid: pDonor-lox66- EGFP WPRE- | Herein | N/A |
| FRT-11 | | |
| plasmid: pDonor-SM_TagBFP2-V5 | Herein | N/A |
| (weakly-fluorescent) WPRE | | |
| plasmid: pDonor-SM_TagBFP2-V5- | Herein | N/A |
| (cyto)-2A-Vcre WPRE | | |
| plasmid: pCag FlEx Vlox SM_FP-myc | Herein | N/A |
| (dark) WPRE | | |
| plasmid: pCag TagBFP2-V5 Cypo PB | Herein | N/A |
| triple miR-E | | |
| shNf1.789:shTrp53.8914:shPten.1524 | | |
| WPRE | | |
| plasmid: pDonor-SM-TagBFP2-V5- | Herein | N/A |
| P2A-SpCas9 WPRE | | |
| Plasmid: pDonor-SM_FP- | Herein | N/A |
| mycBRIGHT- | | |
| pTVl FNLS-Cas9-BW WPRE | | |
| pC0043-SpCas9 BbsI (Empty) crRNA | Herein | N/A |
| backbone (episomal) | | |
| pC0043-SpCas9 sg.Trp53 (episomal; | Herein | N/A |
| for use with FNLS base editor) | | |
| pC0043-SpCas9 sg.Nf1 (episomal; for | Herein | N/A |
| use with FNLS base editor) | | |
| pC0043-SpCas9 sg.Pten (episomal; for | Herein | N/A |
| use with FNLS base editor) | | |
| plasmid: pDonor-SM_FP-myc-P2A-Es- | Herein | N/A |
| pCas9 WPRE | | |
| plasmid: pDonor-SM_FP-myc-P2A- | Herein | N/A |
| Cas 13b WPRE | | |
| plasmid: pDonor-SM_FP-myc-P2A- | Herein | N/A |
| CasRXWPRE | | |
| pU6 BsmBi Empty SpCas9- crRNA | Herein | N/A |
| Cag | | |
| miRFP670-3X-HA WPRE PB | | |
| pU6 BsmBi Empty CasRX- crRNA | Herein | N/A |
| Cag | | |
| miRFP670-3X-HA WPRE PB | | |
| pU6 BsmBi Empty Cas13b- crRNA | Herein | N/A |
| Cag | | |
| miRFP670-3X-HA WPRE PB | | |
| plasmid: pDonorRCE TagBFP2-Hras | Herein | N/A |
| G12V Wpre (RCE donor compatible) | | |
| plasmid: pDonor TagBFP2-Hras G12V | Herein | N/A |
| Wpre (mtmg donor compatible) | | |
| Plasmid: Ubi-EGFP-HRasG12VPB | Breunige et al. Cell | 10.1016/j.celrep.2015.06.012 |
| | Reports, 2015 | |
| plasmid: pDonor- | Herein | N/A |
| SM_FP_Myc_p2a_YAP1-MAM11D | | |
| plasmid: pDonor- | Herein | N/A |
| SM_FP_Myc_p2a c11orf95-RELA | | |
| plasmid: pDonor | Herein | N/A |
| SM_FP_Myc_p2a_Kras G12A | | |
| plasmid: pDonor-H3F3A-K27M-EGFP | Herein | N/A |
| pTV1 Pdgfra D842V COTv1 Trp53-V5 | | |
| WPRE | | |
| plasmid: pDonor-H3F3A-G34R-EGFP | Herein | N/A |
| pTV1 Pdgfra D842V COTv1 Trp53-V5 | | |
| WPRE | | |
| plasmid: pDonor-H3F3A-WT-EGFP | Herein | N/A |
| pTV1 Pdgfra D842V COTv1 Trp53-V5 | | |
| WPRE | | |
| plasmid: pDonor-SM_FP- | Herein | N/A |
| mycBRIGHT- | | |
| pTV1 Pdgfra D842V COTv1 Trp53 | | |
| 270h-P2ACO3-H3F3A K27M WPRE | | |
| plasmid: pDonor-SM_FP- | Herein | N/A |
| mycBRIGHT- pTV1 Pdgfra D842V | | |
| COTv1 Trp53 | | |
| 270h-P2ACO3-H3F3A G34R WPRE | | |
| plasmid: pDonor-SM_FP- | Herein | N/A |
| mycBRIGHT- pTV1 Pdgfra D842V | | |
| COTv1 Trp53 | | |
| 270h-P2ACO3-H3F3A WT WPRE | | |
| plasmid: pDonor-SM_FP- | Herein | N/A |
| mycBRIGHT- pTV1 Pdgfra D842V | | |
| COTv1 Trp53 270h-P2ACO3-H3F3A | | |
| K27M | | |
| WPRE::Ef1a- Akal uc(Inverted) | | |
| plasmid: pDonor-PIP-NLS-Venus- | Herein | N/A |
| P2A- | | |
| mCherry-hGEM1/110 | | |
| plasmid: pDonor-PIP-NLS-Venus- | Herein | N/A |
| P2A- | | |
| mIRFP670-hGEM1/110 | | |
| plasmid: pDonor-PIP-NLS-mIRFP709- | Herein | N/A |
| P2A-mIRFP670-hGEM1/110 (NIR- | | |
| FUCCI) | | |
| plasmid: pDonor-SM_FP- | Herein | N/A |
| mycBRIGHT- pTV1 Pdgfra D842V | | |
| COTv1 Trp53 | | |
| 270h-P2ACO3-H3F3A K27M | | |
| WPRE::Ef1a-NIR-FUCCI (Inverted) | | |
| plasmid: pDonor-SM_FP- | Herein | N/A |
| mycBRIGHT- pTV1 Pdgfra D842V | | |
| COTv1 Trp53 270h-P2ACO3-H3F3A | | |
| K27M WPRE::Ef1a-NIR-FUCCI* (*- | | |
| hGEM C- term NLS mutant; Inverted) | | |
| plasmid: pDonor rtTA-V10-AU1-P2a- | Herein | N/A |
| puro-WPRE | | |
| plasmid: pDonor rtTA-V10-AU1-P2a- | Herein | N/A |
| puro-WPRE TRE-EGFP | | |
| plasmid: pDonor rtTA-V10-AU1-P2a- | Herein | N/A |
| puro-WPRE TRE-EGFP/mDll1 | | |
| Plasmid: pX330-dual U6-p16-p19- | Herein | N/A |
| cdkn2a-Chimeric_BB-CBh- | | |
| eSpCas9(1.1) | | |
| plasmid: pX330-U6-sg.ATRX- | Herein | N/A |
| Chimeric BB-CBh-eSpCas9(1.1) | | |
| plasmid: pX330-U6-sg.AAVS1- | Herein | N/A |
| Chime-ric_BB-CBh-eSpCas9(1.1) | | |
| plasmid: AAVS1-TALENs | Gift: Conklin and | (Mandegar et al., 2016) |
| | Mandegar | |
| plasmid: T7 FlpO-2A-Cre | Herein | N/A |
| plasmid: MC-FlpO-2A-Cre (parental) | Herein | N/A |
| minicircle: MC-FlpO-2A-Cre | Herein | N/A |
| plasmid: CMV Flp-2A-Cre | Gft: Y. Voziyanov | (Anderson et al., 2012) |
| plasmid: mT/mG | Addgene Plasmid | (Muzumdar et al., 2007) |
| | #17787 | |
| plasmid: CAG LF mTFP1 | Gift: I. Imayoshi | (Imayoshi et al., 2012) |
| Software and Algorithms | | |
| Nikon's Confocal NIS-Elements | Nikon | www.microscope.healthcare.nikon.com/ |
| Package | | products/software |
| Imaris 9.1 | Bitplane | imaris.oxinst.com/ |
| ImageJ software | NIH | imagej.nih.gov/ij/ |
| Syglass VR | IstoVisio | www.syglass.io/ |
| STAR/STARlong (version 2.5.1) | Dobin A et al. 2012 | github.com/alexdobin/STAR |
| Cell Ranger software version 2.0.0 | 10X Genomic | support.10xgenomics.com/single-cell- |
| (scRNA-seq) and 3.0.2 (snATAC-seq) | | gene-expression/software/downloads/ |
| Seurat | Butler et al. 2018 | satijalab.org/seurat/ |
| Scanpy | Wolf F A et al. 2017 | scanpy.readthedocs.io/en/latest/ |
| SCENIC (1.0.0-02) | Aibar S et al. 2017 | github.com/aertslab/SCENIC |
| cisTOPIC | Bravo et al. 2019 | github.com/aertslab/cisTopic |
| SnapATAC | Fang et al. 2019 | github.com/r3fang/SnapATAC |
| Harmony | Korsunsky et al. | github.com/immunogenomics/harmony |
| | 2019 | |
| ngs.plot v2.61 | Shen et al. 2014 | github.com/shenlab-sinai/ngsplot |
| IGV v.2.5.0 | Robinson et al. 2011 | software.broadinstitute.org/software/igv |
| bwa-mem | Li, H et al. 2009 | github. com/lh3/bwa |

### Example 2—Results

### MADR Strategy and Reaction Validation

mTmG is a mouse line that constitutively expresses membrane tdTomato and switches to EGFP expression upon Cre-mediated recombination. To effect MADR in mTmG, we created a promoter-less donor plasmid encoding TagBFP2 flanked by loxP and FRT sites (FIG. 1A). We used the minimal 34-bp FRT, which is refractory to Flp-mediated integration, preventing repeated integration at the FRT site. Moreover, the open reading frame (ORF) is preceded by PGK and trimerized SV40 polyadenylation signals (FIG. 1A) to circumvent spurious transcription from unintegrated episomes and randomly integrated whole-plasmids. The ORF is followed by woodchuck hepatitis virus post-transcriptional regulatory element (WPRE), which increases expression, and a rabbit beta-globin pA (FIG. 1A). We crossbred mTmG homozygous and WT mice to generate heterozygous Rosa26^{WT/mTmG} mice (mTmG^{Het}), from which a heterozygous mouse neural stem cells line (mNSC) was derived. We then made two MADR lines by nucleofecting TagBFP2 or TagBFP2-Hras^{G12V} donors (10 ng/μl) and Flp-Cre expression vector (Flp-Cre) (10 ng/μl), each of which was mixed with 3 genetically distinctly colored cells: tdTomato+, EGFP+, and TagBFP2+ (FIG. 1B-C and 8A). A week after nucleofection, FACS analysis indicated MADR efficiency at —1% in the case of TagBFP2. Hras^{G12V} proliferated significantly faster (FIG. 8B). About 5% of TagBFP2+ cells retained either tdTomato or EGFP. More cells retained tdTomato, which can be explained by its slower degradation kinetics (FIG. 8B-C). After another week of culturing the sorted cells, we confirmed the absence of residual EGFP or tdTomato and single-band Hras^{G12V} by western blot, indicating that the recombined Rosa26 locus expressed a single correctly-sized poly-peptide at the aggregate, polyclonal population level without antibiotic selection (FIG. 8D). In order to assess protein production on a per-cell basis, we compared the TagBFP2 protein levels in mNSCs carrying piggybac-TagBFP2 and heterozygous TagBFP2+ MADR cells. The intensity of TagBFP2 in MADR cells had a tight distribution, whereas piggyBac cells had a broad dynamic expression range extending an order of magnitude (FIGS. 1D-F).

To validate the single-copy insertion, we created a donor plasmid carrying puromycin N-acetyl-transferase (PAC) and enriched the cells that correctly express the transgene via antibiotic selection. (FIG. 8E, row 2). We confirmed the correct recombination at Rosa26 locus in these cells (FIG. 8E-G). In selected cells, the tdTomato cassette no longer resided downstream of the CAG-promoter upon dRIVICE, indicating the PAC cells were not tdTomato+ cells actively expressing a promoter-less PAC ORF from unknown chromosomal locations (FIG. 8F, rows 1-2). Additionally, PCR screening revealed the continued presence of the EGFP cistron (though EGFP expression was not detected in these populations [data not shown]) in a small subset of cells, which might happen in a few cells that had Cre-mediated integration but not Flp-mediated excision of EGFP cassette (FIG. 8E, row 4). However, this EGFP cassette was blocked by several polyA elements and situated far downstream from the CAG promoter, which mitigates EGFP expression (FIG. 8F, row 5). To verify this, we used another plasmid carrying TRE-responsive EGFP element (FIG. 8G)). Using this plasmid and selecting for puromycin-resistant cells, we did not observe EGFP fluorescence or expression by western blot, and EGFP expression occurred only with doxycycline (Dox) treatment (FIG. 8H-J).

### MADR-Mediated “One Shot” Generation of Multiple Inducible In Vitro Systems With the Same Genetic Background

Assays for gene function are often performed using transduced or transfected cell lines in vitro, but the constitutive expression of some transgenes can hinder stable cell line generation if the mutations decrease fitness. To avoid this, inducible genetic systems, such as TRE, may be employed to make the cell line first and then start expressing the gene(s) of interest. To showcase the utility of single-allele mTmG^{Het} mNSCs, we established a pipeline for inducible cell line production by nucleofecting these cells with a MADR-compatible vector containing rtTA-V10 and TRE-Bi element (FIGS. 8H). This colorless TRE-Bi-EGFP cell line was enriched with puromycin selection and confirmed using standard in vitro Dox treatment (FIGS. 8I-J).

This in vitro pipeline is beneficial to interrogating the consequences of GOF mutations in various primary cell lines derived from any animal carrying loxP and Frt by providing more homogeneous, inducible stable cell lines. As proof-of-principle for this, and to determine whether the 3′ cistron of the TRE-Bi element was sporadically expressed because of distal promoter/enhancer regions, we generated a cell line that inducibly expresses the Notch ligand, Dll1, with a bi-cistronic TRE-Bi-Dll1/EGFP donor vector (FIG. 8K). This line showed only minute physiological levels of Dll1 without Dox, whereas both EGFP and Dll1 were expressed at similar levels by all cells with Dox treatment (FIG. 8L-M). Notch signaling is one of many molecular pathways that are gene-dosage sensitive, and MADR can be purposed for studying such pathways.

From the mTmG^{Het} mNSCs, we also generated distinct cell lines with 4 different “spaghetti monster” reporter proteins (SM-FPs) in a single nucleofection (Viswanathan et al., 2015). We used this pipeline, which we name MADR with multi-ply-antigenic XFPs (MADR MAX) (FIG. 1G), to assess whether more than one copy of each plasmid could be expressed per cell. SM-FPs were expressed in virtually all cells after antibiotic selection and Dox addition in proportionate ratios (FIG. 1H). Furthermore, we did not observe any cell expressing more than one SM-FP, showing one-transgene-to-one-cell integration (FIG. 1I). This “one-shot” generation of stable, inducible cell lines can thus enable multiplex analysis of multiple transgenes in a common genetic background without causing differential genetic drift during antibiotic selection. We would note that testing MADR plasmids in vivo or with hard to transfect lines can be labor-intensive and thus we have created a host of mouse N2a “proxy” lines of various configurations in addition to the aforementioned mTmG HEK293 and mouse NSCs for in vitro prototyping (FIG. 8N-O).

### MADR Reaction in Human Cell Line

To check that MADR works in human cells, we engineered a MADR-compatible recipient site (FIG. 1J) and using TALENs, we created a human HEK293T cell line with this cassette inserted at the AAVS1 locus. Here, the MADR reaction will replace a CAG-driven tdTomato flanked by loxP and FRT sites (FIG. 1J). To test the function of MADR in human cells, we transfected the cell line with a SM-FP(bright)-myc donor and an alternate TagBFP2-3XFlag donor. Immunofluorescent analysis confirmed that the cell lines that lost tdTomato via excision expressed either the TagBFP2-3Flag or SM-FP(bright)-myc donor transgene (FIGS. 1K-M). These results demonstrate the ability to port MADR to studies involving human cells.

### In Vivo MADR Functional Validation

To effect MADR in vivo, we electroporated (EPed) donor plasmids containing fluorescent protein reporters (TagBFP2 or membrane-tagged SM_FP-myc) and Flp-Cre (0.5 μg/μl each) into the neural stem/progenitor cells lining the ventricular/subventricular zone (VZ/SVZ) of postnatal day 2 (P2) mTmG^{Het} pups (FIG. 2A). Two days after EP, we noted the presence of TagBFP2+ cells along the VZ though some cells expressed detectable EGFP as well (FIG. 9A). At 7 days post-EP, many VZ radial glia and recently-migrated olfactory bulb neurons expressed the SM_FP-myc reporter.

By two weeks, differentiated striatal glia and olfactory bulb neurons appeared (FIGS. 2B and 9B-C). At this time point, we noticed some rare TagBFP2+ cells with persistent EGFP expression at the VZ with the morphological characteristics of ependymal-lineage cells (i.e. multi-ciliated with cuboidal morphology; FIG. 9B). We confirmed that these double-positive cells are indeed Foxj1⁺ ependymal cells (FIG. 9C-G) and noted the inverse correlation between MADR reporter and EGFP. These cells may have minimal levels of protein translation and thus could have slow protein kinetics in general, leading to perdurant EGFP expression. However, most TagBFP2+ cells lacked tdTomato and EGFP expression after the first few days post-EP (FIG. 9B, 2H).

To test the effect of plasmid concentrations on the in vivo recombination efficiencies, we varied the concentrations of Flp-Cre plasmid and SM_FPY-myc for high-sensitivity detection of recombined cells (FIG. 2C and FIG. 9I). We found that increasing recombinase dosages led to increasing EGFP+ cells while higher donor plasmid concentrations had a similar effect (FIG. 2C and FIG. 9I). However, since EGFP and the insertion donor were competing for the same locus, there is a zero-sum effect. Further, due to the perdurance of EGFP, at 2-days many cells expressed both transgenes. Notably, this was likely an inevitable consequence of the half-life of these fluorescent proteins and is similar to the overlap seen between tdTomato and EGFP cells at short survival time points after recombination in the mTmG where the reporter decay was estimated at over 9 days.

To rule out the possibility that transgene expression was due to the expression from randomly integrated or non-recombined episomes, we performed a series of control EPs (FIG. 9J). First, EP of highly concentrated Hras^{G12V} (˜5 μg/μl) and piggyBac (PB)-EGFP reporter into WT pups resulted in no abnormal growth, hyperplasia, or tumorigenesis regardless of Flp or Cre presence (FIG. 9J; for examples of observed phenotypes after MADR of HRas^{G12V} phenotypes see below). In addition, we assessed EPed mTmG pups with Hras^{G12V} harboring an inverted loxP and failed to detect any blue recombined cells or hyperplasia by immunostaining, illustrating the specificity of MADR recombination reaction in vivo (FIG. 9K). Several independent EPs of the Hras^{G12V} donor plasmid and Cre recombinase alone failed to produce tumor formation when examined at 2 weeks post-EP, indicating that Cre cannot induce marked stable integration of MADR donors without Flp-excision (data not shown).

Although MADR is compatible with many existing mice, mTmG presented us with the drawback of being unable to use the red color channel (e.g. FIG. 2B) because of the native tdTomato. We solved this limitation with two methods: by employing a fifth laser channel with >750 nm wavelength fluorophores (FIG. 9L) or by bleaching and immunostaining the now available red channel (FIG. 9M-N). With bleaching, we tested for multiplex labeling of cell lineages in vivo by electroporating 4 SM-FP vectors simultaneously in mTmG^{Het} pups (FIG. 2D). This resulted in four groups of distinctly colored olfactory neurons by 2 weeks, confirming one-transgene-to-one-cell stable integration (FIGS. 2E-F) similar to the in vitro observations (FIGS. 1H-I). These experiments suggest that MADR is a reliable method that depends on a well-known biochemical reaction specifically catalyzed at the target locus. MADR is ideal for expansion microscopy approaches which enable super resolution-like detail of the fine cellular details including astrocytic processes due to the in-creased cell size combined with the excellent signal properties of the SM-FP-myc and EGFP reporters (FIGS. 2G-L).

### Mosaic Analysis with a Tertiary Recombinase (MATR)

One potential limitation of MADR is its utilization of two commonly used recombinases, Flp and Cre. Thus, we tested overlaying conditional VCre-mediated activation of another transgene. To do this, we created a plasmid expressing VCre downstream of TagBFP2-P2A (FIG. 2M). Then we employed an SM-FP-myc-based VCre FlEx reporter (FIG. 2M) to look for recombination with and without TagBFP2-P2A-VCre donor. Notably, SM-FP-myc was not detected when an alternate TagBFP2-3flag was inserted but was readily expressed when the VCre-containing donor was inserted (FIG. 2N-O). Thus, MADR orthogonal recombinases can enable activation of secondary conditional elements.

### MADR to Compare Triple KD vs KO Models

Given the stable genomic insertion and transgene expression that MADR provides, we sought to exploit MADR for generating single-copy in vivo tumor models. LOF tumor suppressor gene mutations such as Nf1, Pten, and Trp53 are some of the most prevalent driver genes in glioma patients. Mouse glioma models show that knocking out these tumor suppressors leads to high-grade gliomas. For example, dual Trp53/Nf1-KOs promote the pre-malignancy hyperproliferation of oligodendrocyte progenitors (OPCs). We wanted to test whether miR-E shRNAs against tumor suppressors are sufficient for tumorigenesis as this approach can be made reversible.

First, we created a donor construct harboring TagBFP2 followed by 3 validated miR-Es targeted at Nf1, Pten, and Trp53 (FIG. 3A). We tested this multi-miR-E construct and observed mRNA-level knockdown efficiency at around 80%, comparable their standard knockdown efficiency (FIG. 3B). We observed the selective overgrowth of TagBFP2+/Pdgfra+ OPCs in vivo. Notably, the EGFP+ population with only Cre-excision yielded a smaller, mixed population of astrocytic cells (FIG. 3C). These recombined EGFP+ cells could serve as an internal control cell population. Notably, we did not detect any tumors at 200 days post-EP, indicating that the complete ablation of Nf1, P53, and/or Pten is necessary for highly penetrant, early-onset tumorigenesis.

To further test this, we switched to CRISPR/Cas9-based knockout of these suppressors. CRISPR/Cas9 has been demonstrated to be highly efficacious for mutating genes in vivo using EP. Using episomal plasmids, we observed that sgRNAs against all Nf1, Trp53, and Pten resulted in the formation of white matter-associated, high grade, Olig2⁺tumors in agreement with GEMM, MADM, and in utero EP-based CRISPR models (FIG. 10C-D). A shortcoming of transposon-delivered CRISPR/Cas9 studies is the lack of a definitive way to lineage trace modified cells because the transposon-delivered Cas9 can catalyze the indels but there is significant chance that the transposon can subsequently “hop” back out, leading to an unlabeled tumor. To address this issue, we created a SM_BFP2-P2A-SpCas9 donor plasmid to simultaneously label and mutate cells, enabling faithful tracing of mutant cells in vivo (FIG. 3D). sgRNAs to target Nf1 and Trp53 were enough to cause terminal morbidity in EPed animals by 5 months, and pathological analysis diagnosed glioblastoma multiforme (GBM). Successful targeting in EPed cells was confirmed by genotyping (FIG. 3E).

Confocal imaging demonstrated that the tumor was largely devoid of tdTomato-labeled populations, whereas the vasculature stayed red (FIG. 3F-F1, 10E). A small EGFP population was observed near where the original targeting site was expected to reside (FIG. 3F, F2; arrowhead). Most of tumor was Olig2+ though CD44+/Olig2-negative regions were observed near the origin site suggesting in situ tumor evolution from proneural to mesenchymal (FIG. 3G-I; arrowhead; 3G2).

To complement these Cas9-based LOF methods, we added the CRISPR/Cas base editor (FNLS) to MADR (FIG. 3J), which catalyzes C-to-T mutation near sgRNA-target site. We introduced SM_FP-myc reporter, FNLS, and sgRNAs de-signed such that they would create premature stop codons in Nf1, Trp53, and Pten (FIG. 3K). Amplicon sequencing of GFP-sorted MADR cells confirmed that the base editors could induce premature stop codons (FIG. 10F). Two months later, we noted a dramatic expansion of OPCs similar to the mir-E and Cas9 LOF studies (FIG. 3L-M). All of these KD vs KO studies were done in the same mouse line (mTmG) and demonstrated MADR's various means for multiplexing LOF analysis with combined lineage tracing. Moreover, we have generated MADR elements for CRISPR/Cas variants for gene knockdown/knockout (FIG. 10G).

### GOF Oncogene Dosage Sensitivity Revealed by MADR

We made a Hras^{G12V}-based MADR donor compatible with RCE reporter mouse and performed in utero EP (IU-EP) in E14 RCE-heterozygous embryos (FIG. 4A-B). PiggyBac-mediated Hras^{G12V}-overexpression in mouse embryos has been shown to induce high-grade tumors within 15-20 days of birth (Glasgow et al., 2014). In contrast, we did not observe tumor growth when the MADR×RCE-het animals were examined at P15. However, we noted a marked cell-fate switch of TagBFP2-Hras^{G12V} cells to the astroglial lineage (FIG. 4C, 11A). EGFP+ Cre-excised cells consisted of a mixed population of neurons and glia (FIG. 4C, 11A). This is an important case where MADR disagrees with multicopy-transgene based transposon models, highlighting the consequence of GOF oncogenes depending on gene dosage. Besides the mTmG and RCE lines, MADR can be employed with any off-the-shelf GEMM harboring dual recombinase sites, including Ai14, R26-CAG-LF-mTFP1, Ribotag lines and the thousands of IKMC mouse lines using a splice acceptor to investigate the effects of substituting transgenes under the native cis-regulatory sequences (FIG. 11B).

We previously studied a PB-tumor model based on Hras^{G12V}, which results in 100% penetrant glioma when EPed in post-natal WT pups. When the MADR TagBFP2-Hras^{G12V} transgene was delivered postnatally to mTmG^{Het}, Hras^{G12V}+ cells similarly overproliferated when compared with EGFP+populations (FIG. 11B-C). To definitively examine the effects of Hras^{G12V} dosage, we EPed Hras^{G12V} in homozygous mTmG, in which we expected to be able to differentiate Hras^{G12V}×1 or Hras^{G12V}×2 cells (FIG. 4D-E). All mice rapidly developed glioma and reached terminal morbidity within 3-4 months (data not shown).

Interestingly, in homozygous mTmG mice, blue-only cells (Hras^{G12V}×2) occupied a bigger patch of tumor cross-section than cells expressing both blue and green (Hras^{G12V}×1) (FIG. 4F-G). Using PB-EP, we also observed that the patch of brighter EGFP-tagged Hras^{G12V} cells expressed phosphorylated Rb1 (pRb1) more than the dimmer EGFP+ cells (FIG. 11D). In MADR, where the copy number is unambiguous, most of the Rosa26^{HrasG12V×2} cells seemed to express pRb1, whereas it was expressed in fewer hemizygous Rosa26^{HrasG12V×1} cells (FIG. 4G-H). MADR mosaics enable one to genetically distinguish these two groups of cells and examine their differences, whereas PB tumor models cannot, and confirms that the copy number of oncogenes—which is uncontrollable in many somatic transgenic methods—can significantly alter the profile of resulting tumors.

### MADR Ependymoma Models Based on Fusion Proteins

Many tumor drivers are fusion proteins, but it can be difficult to make a conditional GEMM mimicking chromosomal rearrangement. For example, the fusion protein drivers YAP1-MAML1D and C11orf95-RELA are recurrently seen in supratentorial ependymomas, and we made MADR vectors to express them (FIG. 4I). Compared to MADR-KrasG12A tumor models—a genetic driver of glioma, YAP1-MAML1D and C11orf95-RELA MADR tumor cells showed remarkably different initiation patterns. Whereas KrasG12A cells rap-idly invaded the striatum and proliferated (FIG. 11E), YAP1-MAML1D tumors delaminated into rosette-like structures and induced a non-cell autonomous reactive gliosis in the surrounding EGFP+ control cells (FIG. 11F-G). C11or95-RELA cells displayed a mixed phenotype, whereby they often stayed along the VZ wall or formed small clusters near the ventral VZ (FIG. 11H-I). To mimic the coincident loss of Cdkn2a that is frequently seen in ependymomas, we used Cas9 with sgRNAs against p16 and p19. YAP1-MAML1D×p16/19-KO animals reached terminal morbidity within roughly 1.5 months (FIG. 4J-K). However, the C11orf95-RELA×p16/19-KO tumors showed a more protracted survival, reaching terminal morbidity at approximately 3 months (FIG. 4K-L). Unlike the infiltrative margins of our glioma models and human glioma, the ependymomas exhibited defined margins with a lack of invading cells (FIG. 11J-K) akin to pushing margins seen in patients Taken together, this data demonstrated MADR's ability to model diverse tumor types, including those driven by fusion proteins.

### Direct Comparison of H3f3a G34R and K27M Pediatric Glioma Drivers Using MADR

Almost all human tumors present with a distinct set of somatic and germline mutations, either passenger or directly contributing to cancer. With the ability to pick and choose mutations and to compare these sets of mutations, MADR can serve as a personalized tumor model platform tailored for studying nuanced idiosyncrasies with important implications to drug resistance and survival that are unique to each tumor subtype. As a proof-of-principle, we chose to model pediatric GBM where H3F3A K27M or G34R mutations are observed in more than 50% of patients, but co-occur with a variety of other mutations. For example, H3F3A mutations are often coincident with recurrent dominant-active Pdgfra (D842V), and dominant-negative Trp53 (R270H) To demonstrate MADR's utility in this context, we made donor plasmids for modeling simultaneous H3f3a, Pdgfra, and Trp53 mutations—with variants differing only by missense mutations for G34R or K27M to study the differential effects of these driver genes (FIG. 5A).

First, we checked for appropriate expression of H3f3a, Pdgfra, and Trp53 by immunohistochemistry in vivo and in vitro and noted coincident expression of all proteins (FIG. 12A-B). Next, we introduced these plasmids by postnatal EP into sibling pups over several litters. To transfect the stem/progenitor cells in both cortical and striatal VZs, the electrodes were swept as shown (FIG. 5B-C). For the first 2-4 months, there was a diffuse expansion of EGFP+cells in both G34R and K27M mice but no tumors were identifiable by clinical pathology (FIG. 12C), similar to the extensive pretumor phase seen with MADM glioma models.

Patient tumors bearing either K27M or G34R/V mutations exhibit different transcriptomes as well as clinical features. Human K27M gliomas cluster along the midline, whereas G34R occur in the cerebral hemispheres. K27M tumors manifest in younger patients than G34R/V. Seemingly in agreement with their earlier clinical presentation, some K27M+ mice exhibited midline gliomas by P100, at which time G34R+ displayed diffuse glial hyperplasias and very rare, small tumors (FIG. 5D-E and data not shown). At P120, K27M tumors predominantly localized to the sub-cortical structures but cells could be observed in the white matter tracts with a few cells in the deeper cortical layers (FIG. 5F). In contrast, G34R tumors localized to the corpus callosum and deeper cortical layers, often forming “butterfly” gliomas across the midline (FIG. 5G) in a pattern akin to the hemispheric localization seen in patients. This happened despite the aforementioned targeting of the striatal VZ (and observable hyperplasia of some of these cells; yellow arrow in FIG. 5G).

Pathological features included high cell density, microvascular proliferation, and necrosis at late stages (FIGS. 5H-J). Both K27M, and G34R tumors were 100% penetrant and showed accelerated endpoints compared with H3f3a WT tumors containing Pdgfra and Trp53 mutations (FIG. 5K), but consistently exhibited a tumor “site-of-origin” (i.e. midline vs. cortical) matching to their patient counterparts (FIG. 5L). To ascertain the expression of the appropriate H3f3a mutation we employed monoclonal antibodies against the respective mutant residues with no cross reactivity (FIG. 12D-G).

To compare the cell autonomous properties of these cells we exploited unique properties of MADR whereby each allele can receive only one transgene insertion, and co-delivered K27M and G34R plasmids at a 1:1 ratio (FIG. 5M-N). The use of the aforementioned anti-K27M and anti-G34R antibodies in serial sections confirmed the co-expression of the respective transgenes (FIG. 5O-P) in individual tumors. Further, using a biotin-conjugated K27M antibody and rabbit serum-mediated blocking to allow for simultaneous G34R mutant cells, we confirmed that each SM_FP-myc+ cell expressed only one H3f3a mutant variant (FIG. 12H). Quantification of K27M and G34R cells demonstrated a highly significant increase in K27M, indicating their ability to out proliferate their G34R counterparts (FIG. 5Q). These findings indicate that the K27 and G34 residues given the same genetic background—or even animal—can alter the time and location of onset of these glioma subtypes similar to human phenotypes.

Several studies have shown that K27M mutations lead to hypomethylation at the H3K27 residue, and we confirmed the hypomethylation of K27M mutant cells by H3K27me3 antibody (FIG. 12I-J). The invasive tumor cells exhibited perineural satellitosis as has been described in human K27M tumors, and the juxtaposed EGFP+ K27M glia and neurons showed markedly different H3K27me3 levels at high resolution (FIG. 12K). Hypomethylation was not an artifact of tumor growth because in our CRISPR/Cas9-based Nf1/Trp53-KO models, gliomas were normal or hypermethylated (FIG. 12L). Acetylation at H3K27 did not seem grossly altered (FIG. 12M-N).

### MADR K27M Recapitulates Human Tumor Heterogeneity and Developmental Hierarchy

Immunohistological analysis demonstrated that tumor cells upregulated Bmi1 (FIG. 12O-P), which had recently been identified as being enriched in K27M glioma. As a population, K27M cells broadly expressed glial marker such as Aldh1l1—a canonical marker of astroglial lineages. Aldh1l1 co-localized with EGFP+ tumor cells most prominently at the margins of the tumor (FIG. 12R). These cells tended to have a larger size, akin to reactive astrocytes. Conversely, NG2-labeled EGFP+ cells tended to be smaller, with morphologies similar to OPCs (FIG. 12S). To enable future non-invasive imaging and observation of tumor progenitor dynamics, we generated secondary constitutive cistrons for both non-invasive imaging, and cell cycle phase reporting with FUCCI (FIG. 12T-V). Further, MADR naturally lends itself to separating normal and tumor populations by the fluorescent markers (FIG. 12W). We used this feature to demonstrate that of two previously identified kinase inhibitors— Akt1/2 inhibitor and Vacquinol-1—that were found to be selectively toxic to K27M tumor cells; the Akt1/2 inhibitor similarly inhibited NPC proliferation (FIG. 12X). Our confirmation that Vacquinol-1 does not alter NPC culture growth yet inhibits K27M growth provides evidence for continued investigation of this compound in the context of these tumors.

This heterogeneity of glial markers was ostensibly similar to recent findings in human K27M tumors, which demonstrated a significant degree of intratumor heterogeneity by single-cell RNA sequencing (scRNA-seq). Given the availability of this analogous human K27M data we took the unique opportunity to credential the MADR model cells against their human counterparts and gain deeper insight in to the heterogeneity through the use of scRNA-seq.

We subjected EGFP+ sorted tumor cells from 3 independent K27M tumors to droplet-based scRNA-seq (FIG. 6A, Table 2). Copy-number variation (CNV) analysis demonstrated chromosomal abnormalities (FIG. 13A) as is observed in human K27M glioma. Following sequencing, alignment, and quality control, we clustered the mouse K27M cells using Seurat. For the choice of gene set for CCA-alignment, we used the four programs termed P1-4 that were identified in the human dataset as this dataset and associated analysis represented a unique opportunity to credential our tumors at single-cell resolution against their human counterparts (FIG. 6B-C, 13B-D).

TABLE 2
Mouse Tumor Samples

| Type of | | | Time | Cell Line | ChIP- | |
|---|---|---|---|---|---|---|
| Sample | Protocol | Area | from EP | Created* | Seq | pDonor variant |
| K27M-1 | 10X 3′ | Disseminated | 150 days | X | X | pDonor-H3F3A-K27M- |
| | scRNA-seq | | | | | EGFP pTV1 Pdgfra D842V |
| | | | | | | COTv1 Trp53-V5 WPRE |
| K27M-2 | 10X 3′ | Striatal | 106 days | X | X | pDonor-SM_FP- |
| | scRNA-seq | | | | | mycBRIGHT- pTV1 Pdgfra |
| | | | | | | D842V COTv1 Trp53 270h- |
| | | | | | | P2ACO3-H3F3A K27M |
| | | | | | | WPRE |
| K27M-3 | 10X 3′ | Striatal | 149 days | | X | pDonor-H3F3A-K27M- |
| | scRNA-seq | | | | | EGFP pTV1 Pdgfra D842V |
| | | | | | | COTv1 Trp53-V5 WPRE |
| K27M-4 | 10X | Disseminated | 222 days† | X | | pDonor-SM_FP- |
| | snATACseq | | | | | mycBRIGHT- pTV1 Pdgfra |
| | | | | | | D842V COTv1 Trp53 270h- |
| | | | | | | P2ACO3-H3F3AK27M |
| | | | | | | WPRE |
| K27M-5 | 10X | Striatal | 251 days† | | | pDonor-SM_FP- |
| | snATACseq | | | | | mycBRIGHT- pTV1 Pdgfra |
| | | | | | | D842V COTv1 Trp53 270h- |
| | | | | | | P2ACO3-H3F3AK27M |
| | | | | | | WPRE |

*Cell lines created from parallel processing of additional GFP+ cells. All 10X scRNA- or snATAC-sequencing was done acutely from the dissociated brain tissue.
†Initial EPed population size was decreased compared with typical results in this group leading to increased tumor formation span

The “Cycle” cluster consisted of cells expressing markers of proliferation, including Top2a, mKi67, and Ccnb1 (FIG. 6B-C; FIG. 13E). AC and OC clusters expressed genes associated with more differentiated astrocytes and oligodendrocytes, respectively (FIG. 6B-C; 13D), while the largest cluster, termed “OPC” based on the human P4 cluster, expressed genes including Olig1, but did not seem to clearly fall into a differentiated cell lineage (FIG. 6B-C; 13D). Scoring clusters based on gene lists identified in human K27M con-firmed the enrichment of astroglial markers in AC and the enrichment of oligodendroglial markers in OC (FIG. 6B-D).

To conduct cross-species analysis of K27M gliomas, we repeated the Seurat clustering with all the cells from mouse and human K2M tumors (FIG. 6E-G; 13F-I) and saw that the 9 combined single-cell datasets continued to yield the four clusters seen in the individual mouse and human CCA alignments (FIG. 6H-J). By splitting the combined 9 sample UMAP into each respective sample, we noted relatively similar—though not uniform—contributions of cells from each sample to each individual cluster (FIG. 6J; 13I). Our specific combination of mutations closely matched patient MUV10, and this patient contained less AC cells than other patients, as our mouse K27M cells did (FIG. 13M).

We also performed clustering with the more common practice of employing highly-variable-genes for CCA, clustering, and UMAP analysis. This approach led to some almost identical clusters (e.g. cycling populations) but division of other populations into sub-clusters (e.g. OPC), which varied by the parameters chosen (FIG. 13N). This variability of clustering is an inherent issue in scRNA-seq due to batch effects, patient-specific transcriptome alterations, and in challenges associated with cross-species comparison.

We also used the differentially expressed genes identified across human K27M, GBM, IDH astrocytoma, IDH oligodendroglioma to plot a heatmap comparing our 3 mouse K27M tumors. Our MADR K27M tumors were more similar to the human counterparts than to other glioma subtypes (FIG. 6K). Further, human K27M cells are characterized by a high proportion of cycling cells, as our mouse tumors did (FIG. 6L).

### MADR K27M Regulatory Network Analysis

We have shown a global matching between the MADR-based K27M mouse and the human K27M glioma transcriptomes, especially in that they show similar developmental hierarchies and overrepresentations of cycling cells. To our knowledge, our K27M scRNA-seq dataset is one of the first created to validate a mouse tumor model. Therefore, we subjected the datasets to further analysis to gain novel insights. The K27M mutation leads to widespread epigenetic perturbation, which led us to focus on whether similar transcription factor (TFs) networks underlie human and mouse tumors.

SCENIC is a method that applies random-forest regression to scRNA-seq datasets to identify regulons (a regulon is a curated, known co-expression module based on a TF and its positively correlated target genes). This type of regulon-based analysis is robust because of its holistic nature, and minimizes the batch and patient-specific effects, which can confound scRNA-seq (FIG. 14A-J).

In tSNE-plots derived from parallelly processing the mouse and human K27M cells in SCENIC, the cells were clustered along their cell types, indicating that these cell clusters have differential TF-networks (FIG. 7A-B). We observed that the cycling cells in both our model and human data showed the enrichment of E2F family modules (E2F1, E2F7, E2F8), EZH2, MYBL1, and BRCA1 (FIG. 7A-D). These TFs have no significant differential expression among cell clusters, indicating that their activity is largely not transcriptionally regulated (FIG. 7E-F). EZH2 is diagnostically and functionally associated with K27M mutations. MYBL1 is a driver gene in pediatric gliomas, indicating its functional importance. E2F members are known to act in concert, especially during the embryonic stages. Given the dramatically enhanced activity of these proteins in the mitotic clusters, we decided to look for additional cell-cycle associated gene networks that might not be found in the SCENIC regulon sets. GBMs and K27M pediatric gliomas are characterized by poorly differentiated cell classes. NANOG, OCT4, SOX2, MYC2, and Embryonic Stem-expressed (exp1) gene sets and the under-expression of PRC2, SUZ12, EED, and H3K27-bound gene sets have shown to indicate this poorly differentiated state (FIG. 7G-H). In both human and mouse datasets, this embryonic stem-signature seemed to be strongest in the cycling cell types (FIG. 7G-H). As a further evidence, we performed Chip-seq on the three tumors, identified the genes that are specifically hypomethylated, and found that this subgroup of genes is highly expressed in the cycling cells (FIG. 7G; 14K-M).

To examine the underlying epigenetic state through the examination of differentially accessible genome regions (DARs), we performed single-nucleus ATAC-seq of K27M mouse tumors and compared them to normal P50 and E18 mouse brains (FIG. 7I-L, FIG. 14N-W). While the P50 brain exhibited well-spaced, canonical marker gene defined clusters FIG. 7I, FIG. 14N-O); both the E18 brain (an alignment of 3 independent datasets; FIG. 7K; FIG. 14P-S) and tumor cells (but not the co-captured tumor microglia—which create distinct clusters) exhibited less well-defined DARs (FIG. 7L, FIG. 14T-W). Moreover, pathway analysis of K27M tumor clusters (FIG. 7M) was notably altered when compared with the pure P50 astrocyte and OPC clusters (FIG. 14Y), including a BRCA1-associated term consistent with the SCENIC findings.

Finally, alignment of DARs from these scATAC samples and analogous bulk datasets further supported the tSNE findings that glial lineage-associated transcription factors like Olig2, Sox9, and Sox10 exhibit reduced relative accessibility when compared with P50 glial lineages and mutual exclusivity in terms of Sox9 and Sox10 (FIG. 7N). The K27M scRNA-seq data was consistent with this as Sox9 and Sox10 mRNA were co-expressed in each tumor cluster and often in individual cells, which is exceedingly rare in the normal adult brain. However, DARs found in the bulk samples were recapitulated in the scATAC datasets (i.e. Cacng8 in K27M tumors—6.322 log2 ratio K27M:NPCS and Hes5 in NPCs—3.248 log2 ratio NPC:K27M tumors; FIG. 14N). Further, co-captured microglia retained robust DARs, arguing against dominant batch effects; FIG. 7N). Finally, the K27M tumor cells exhibited a preponderance of immediate early gene motifs associated with cancer and motifs for many of the ES-associated TFs (FIG. 14Z) previously identified in aggressive tumors. Taken together, the K27M oncohistone leads to altered activity of a subset of TFs in the actively cycling subsets of these tumors by generating a primitive epigenetic state.

### Example 3

We designed two AAV viruses. One expresses FlpO-2A-Cre while the other has a non-expressed (inverted) TagBFP reporter gene. When the TagBFP is transduced into cells by itself, it doesn't appear to be expressed. However, in the presence of the FlpO-2A-Cre virus, cells with the MADR recipient locus appear to lose expression of the tdTomato and EGFP transgenes and begin to express TagBFP (FIG. 26).

The significance of this is because it would obviate the need for proliferation to facilitate MADR and thus make it easy to target postmitotic cells and other tissues with single-copy transgenesis. Many types of disease models or safer gene therapy dosing can thus be made.

### Example 4

We modified AAVS1-pAct-GFPnls to AAVS-pACT-loxP-TagBFP-V5-nls WPRE FRT and have MADR-ready iPSCs. The function of this MADR cassette was validated in HEK293T cells (FIG. 27) and thus, we are able to exchange pDonor transgene elements in induced pluripotent stem cells (iPSCs) and sublineages.

### Example 5

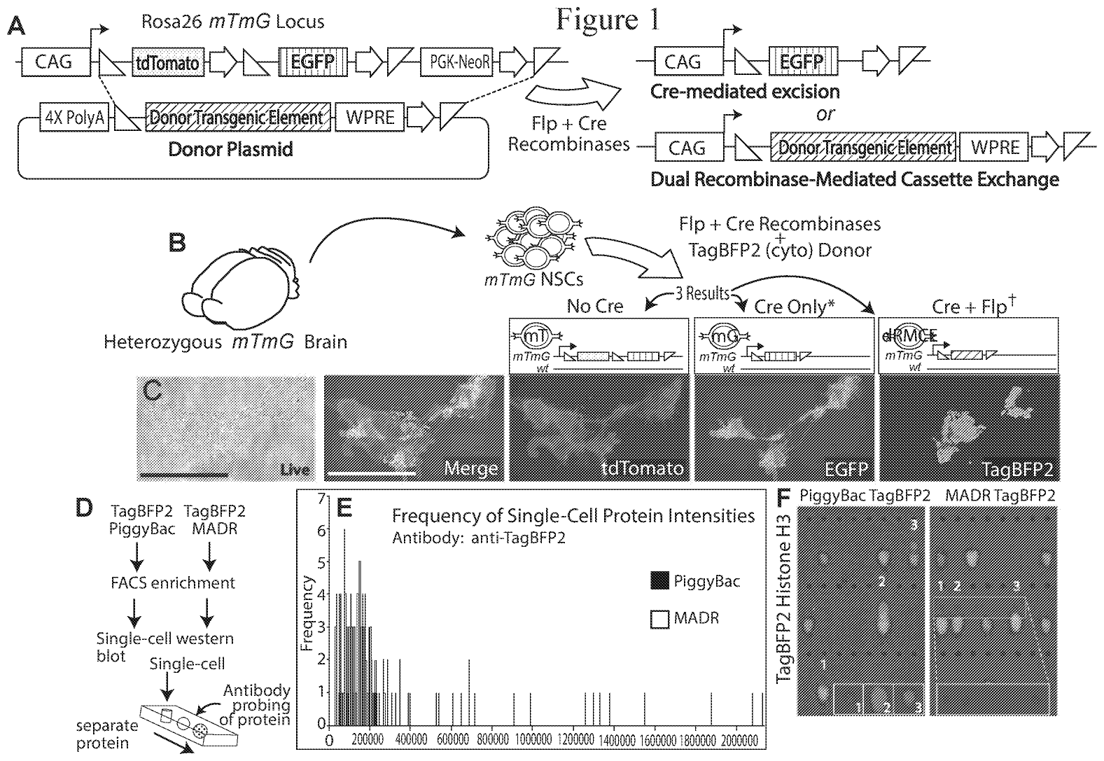

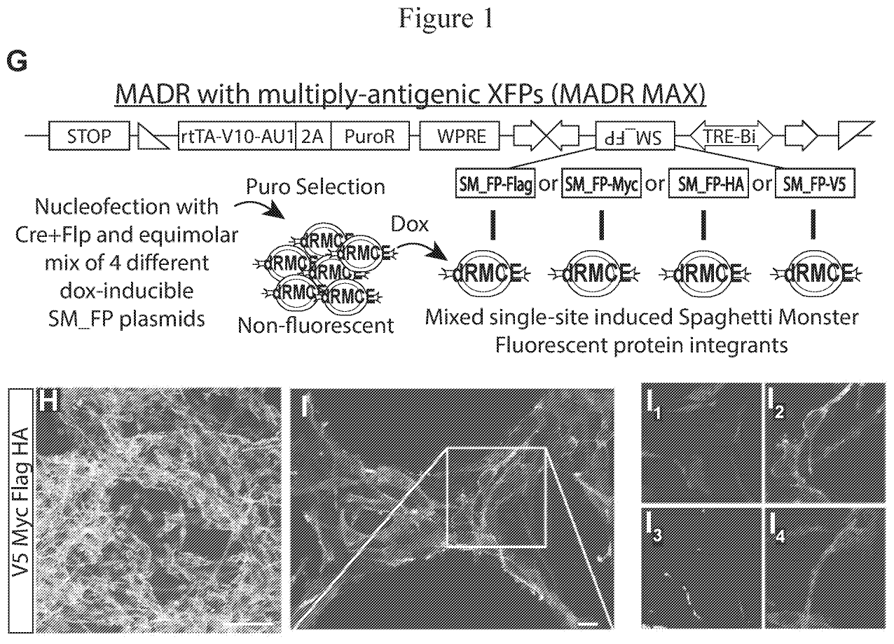

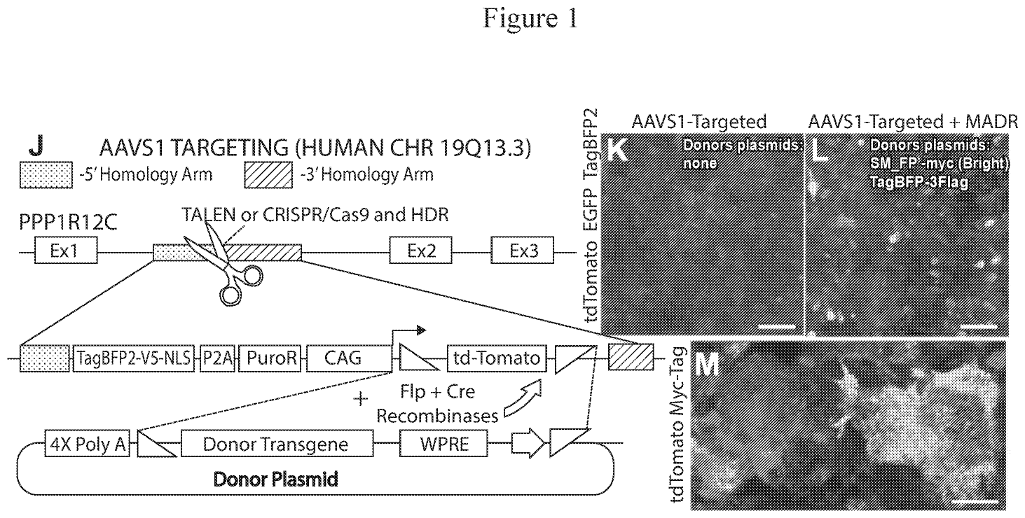

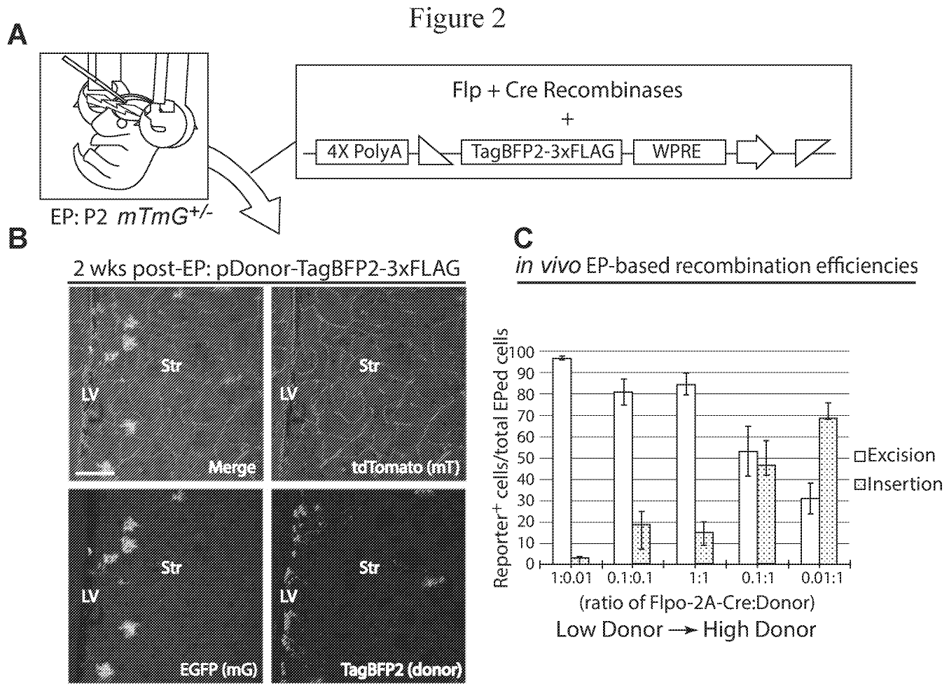

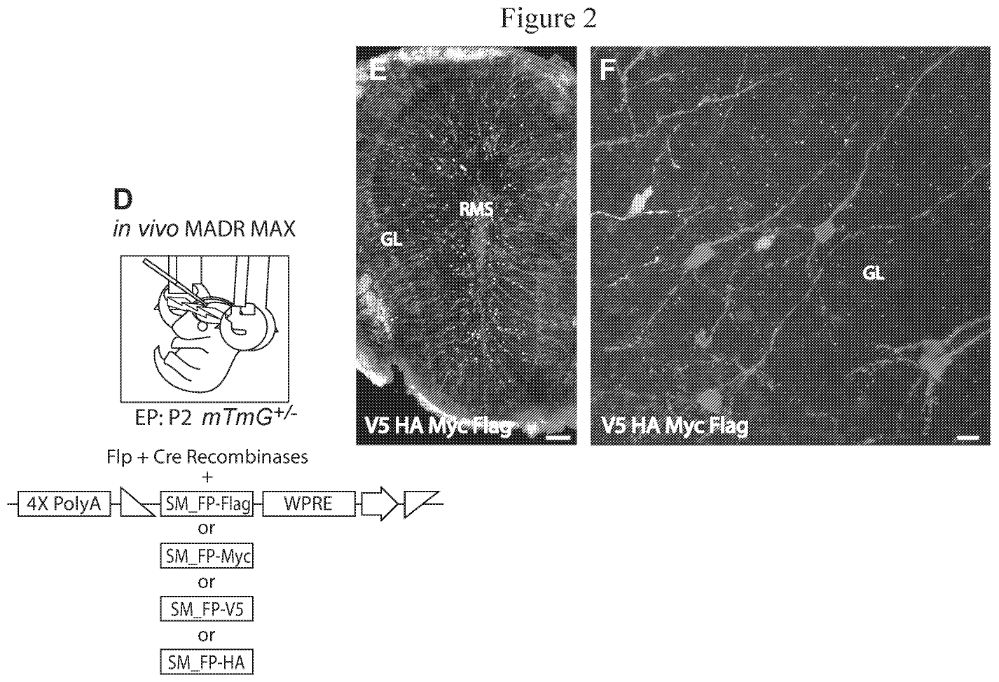

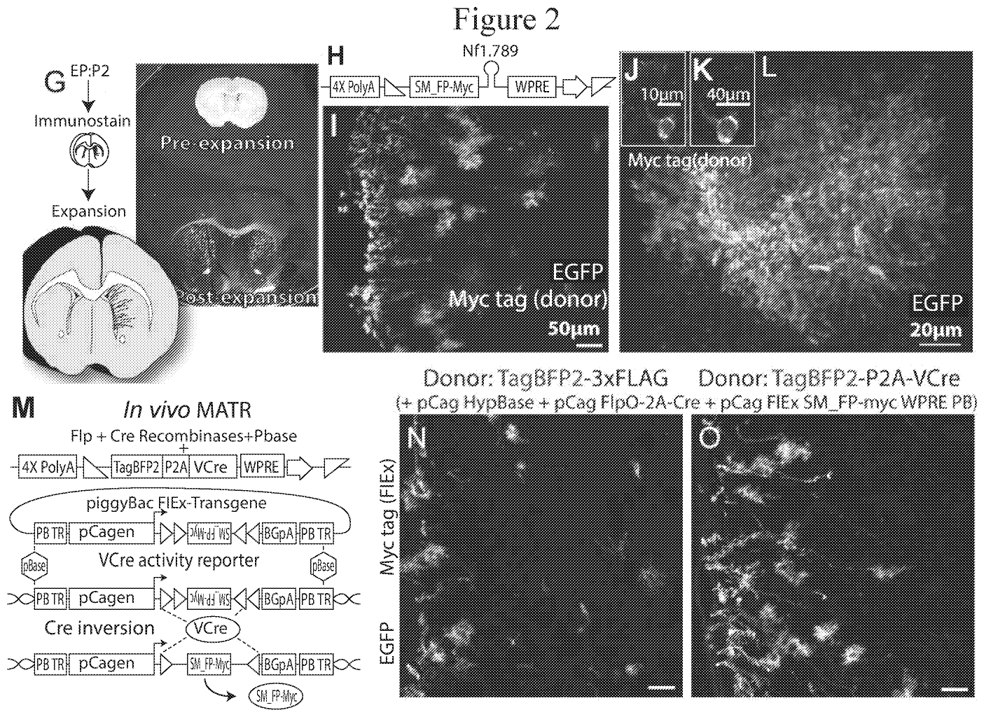

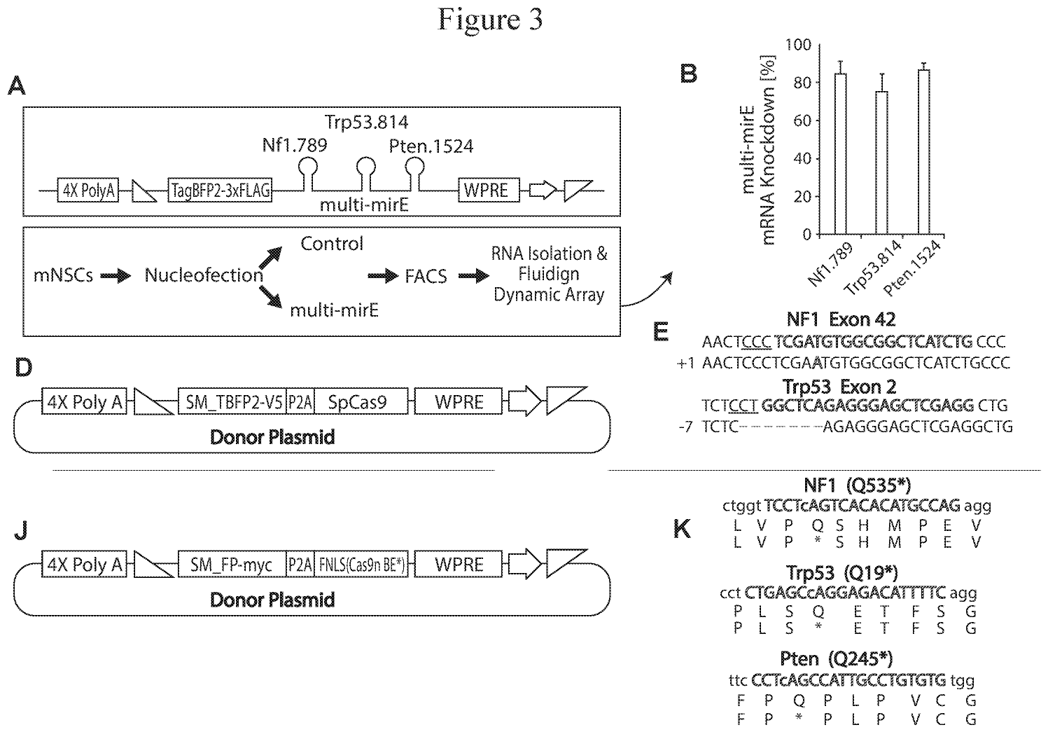

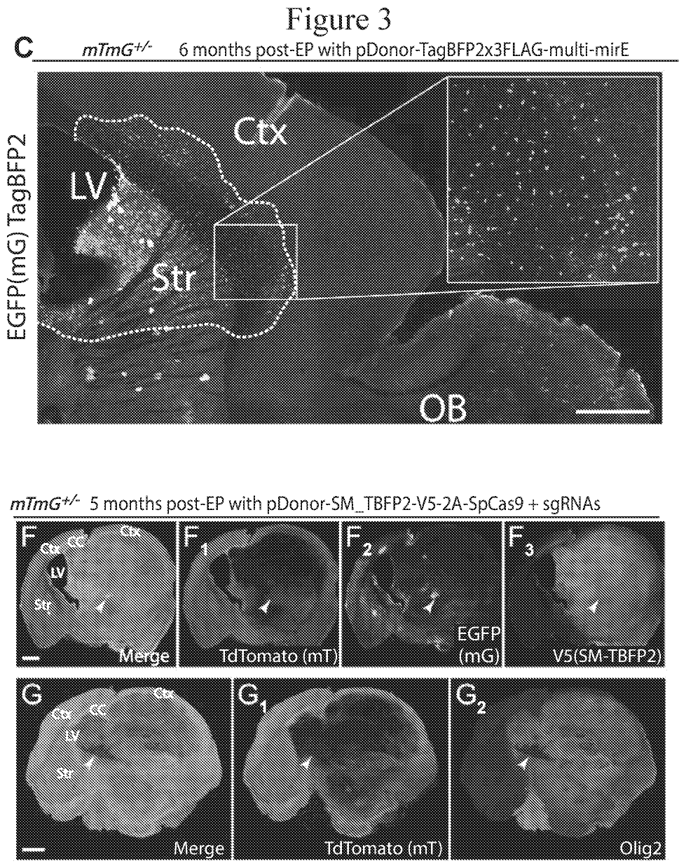

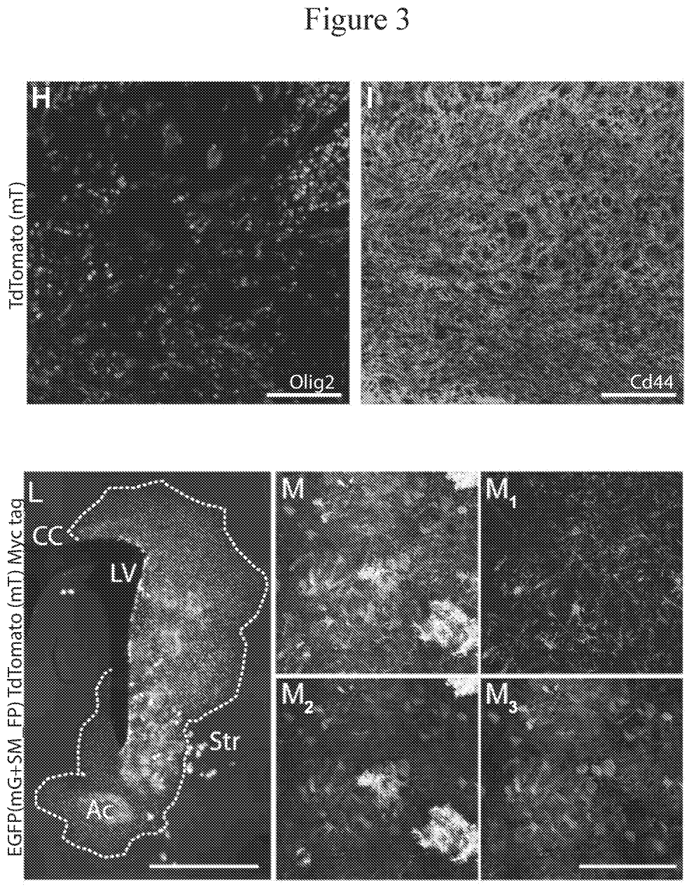

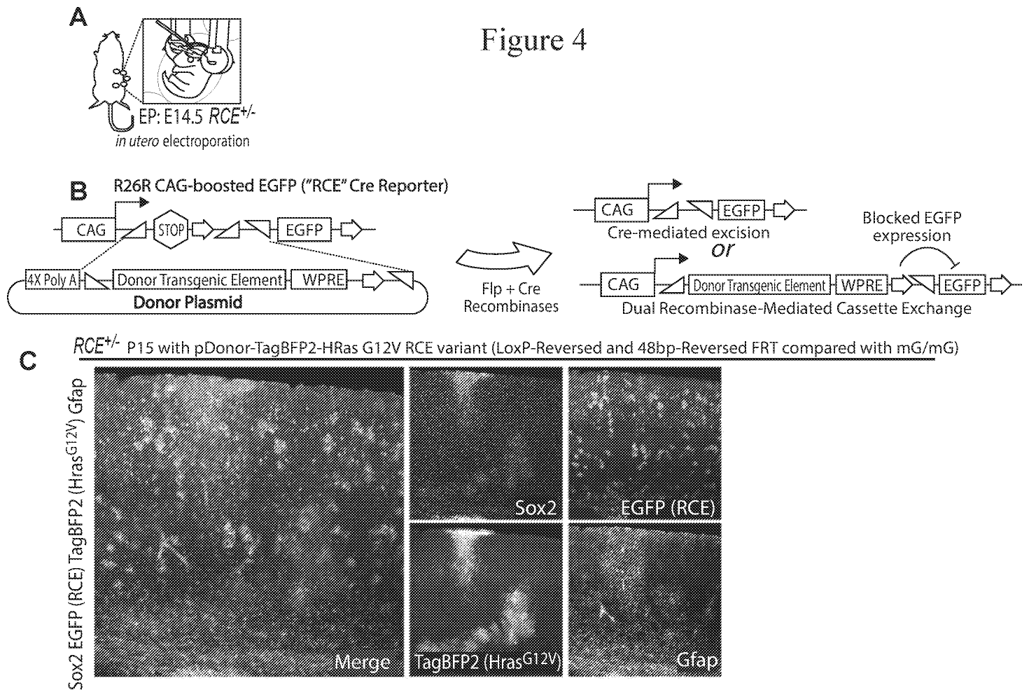

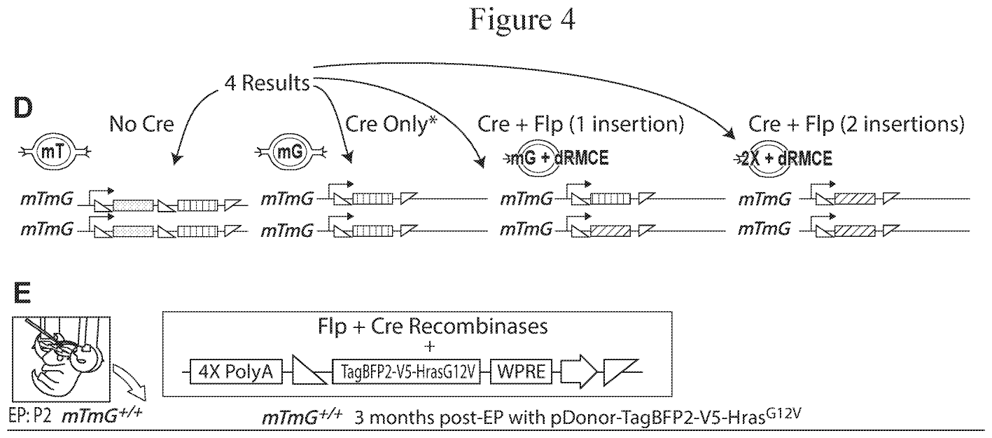

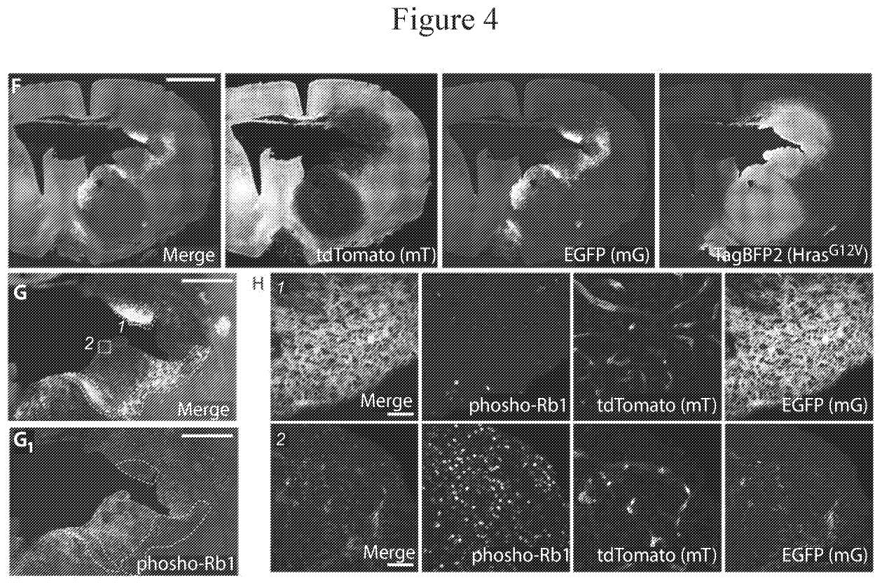

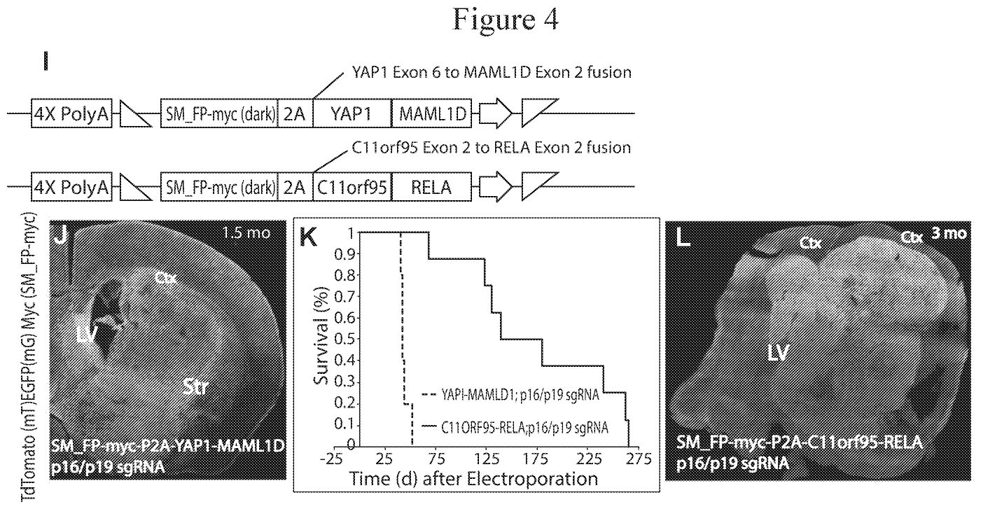

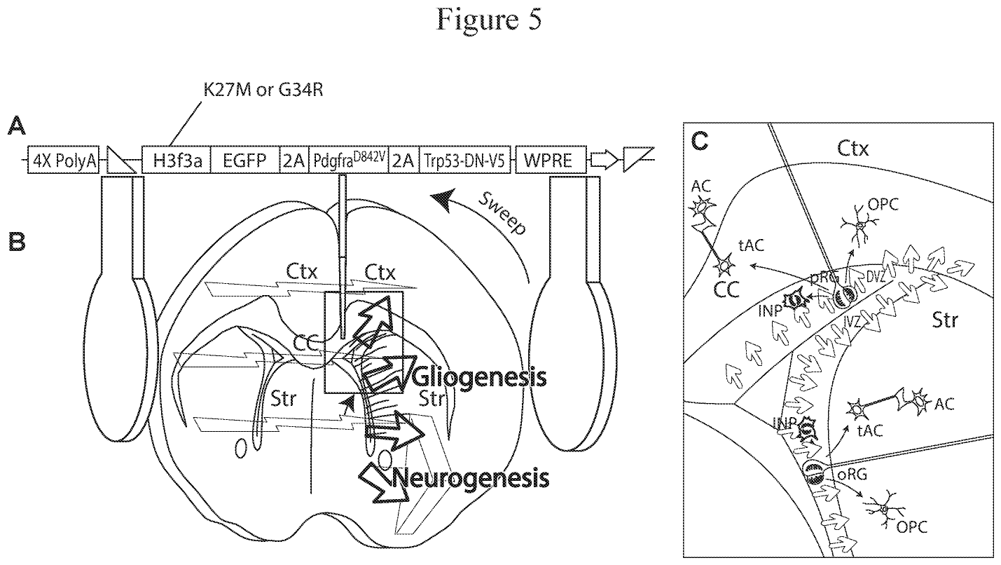

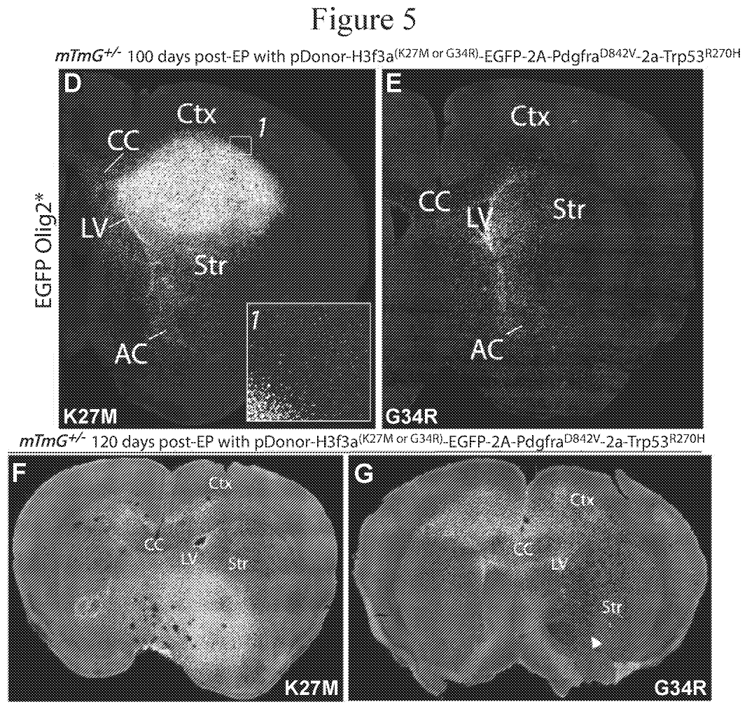

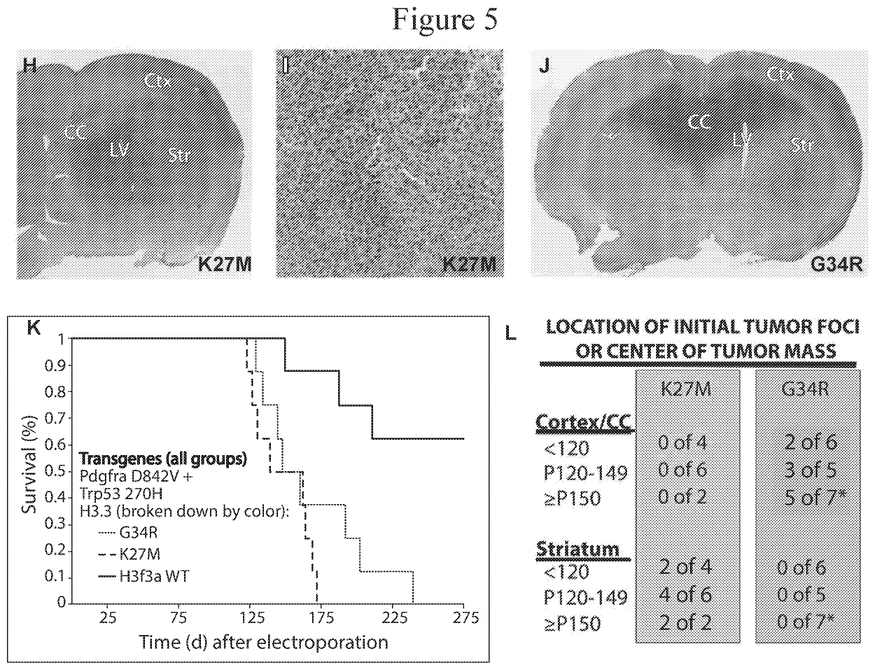

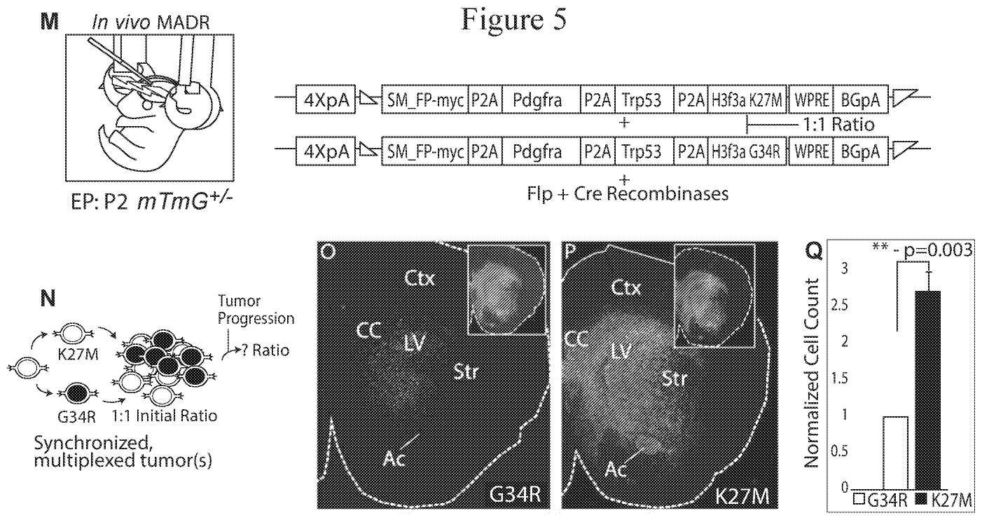

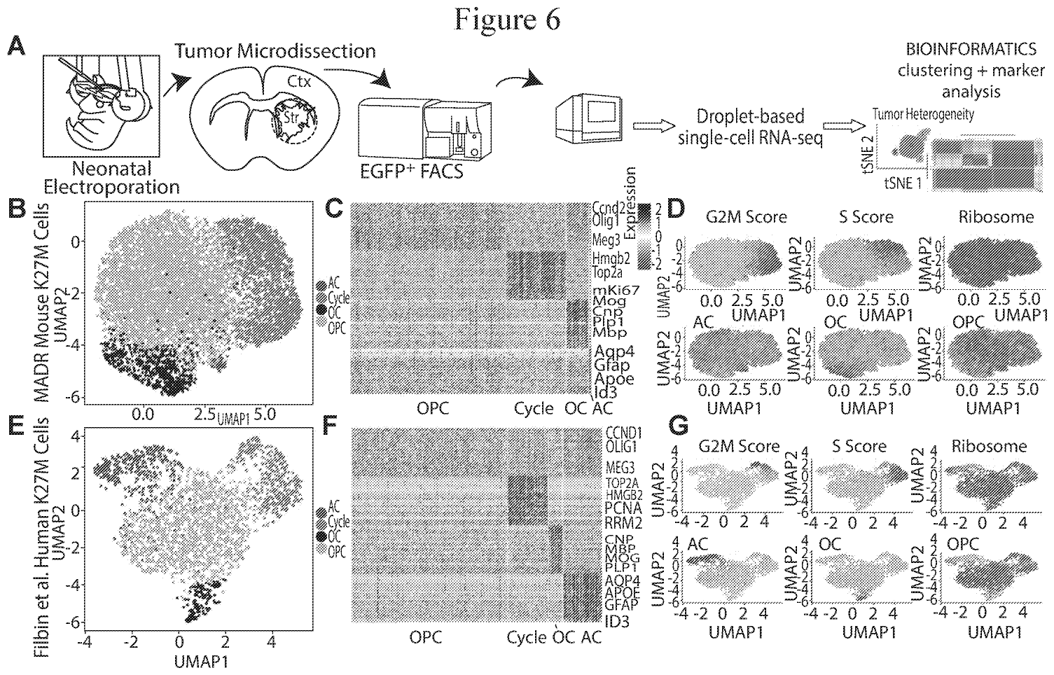

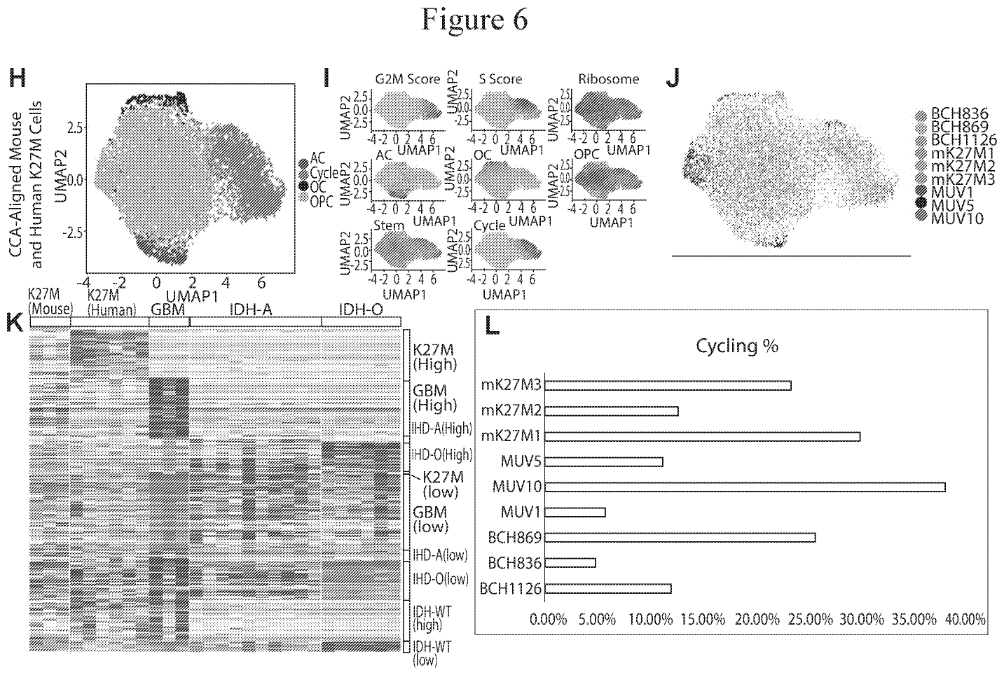

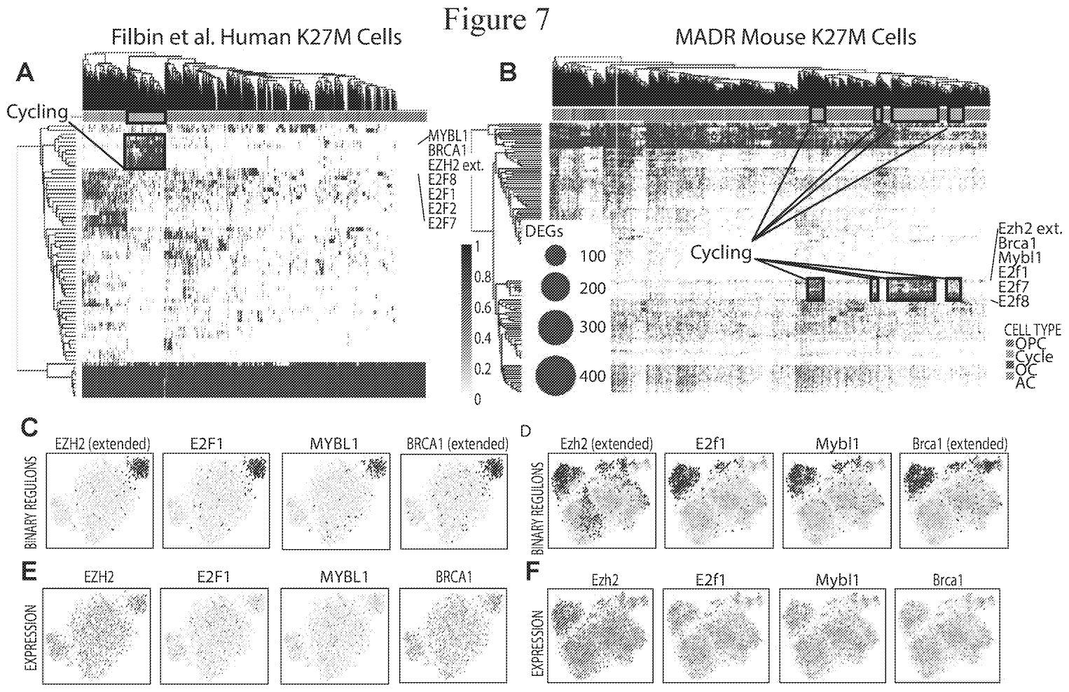

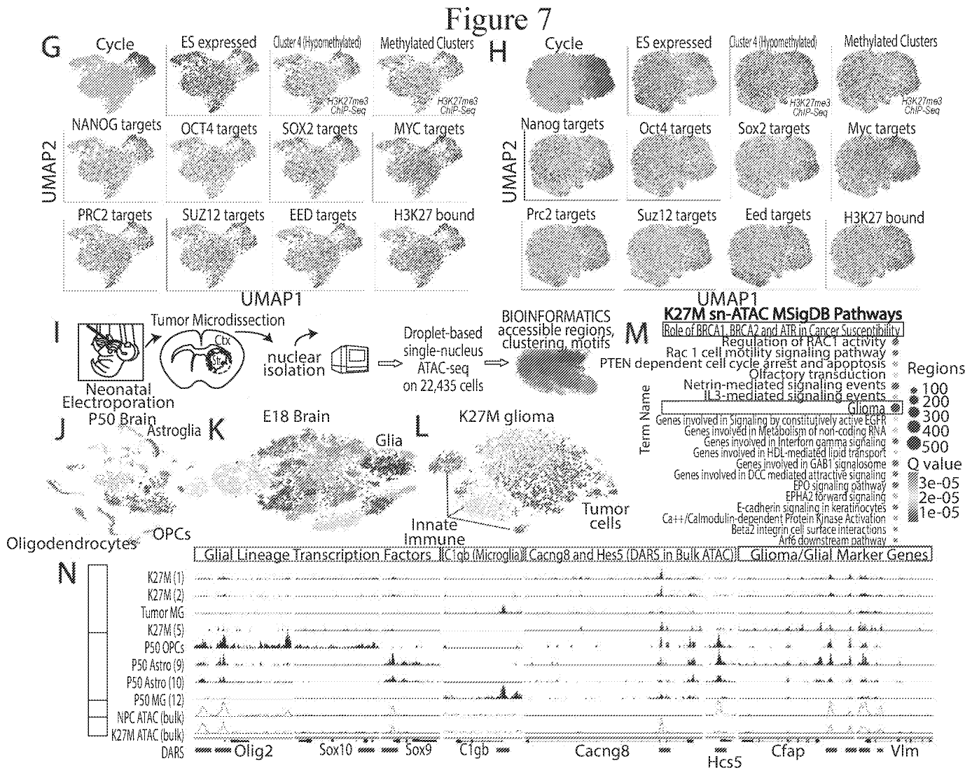

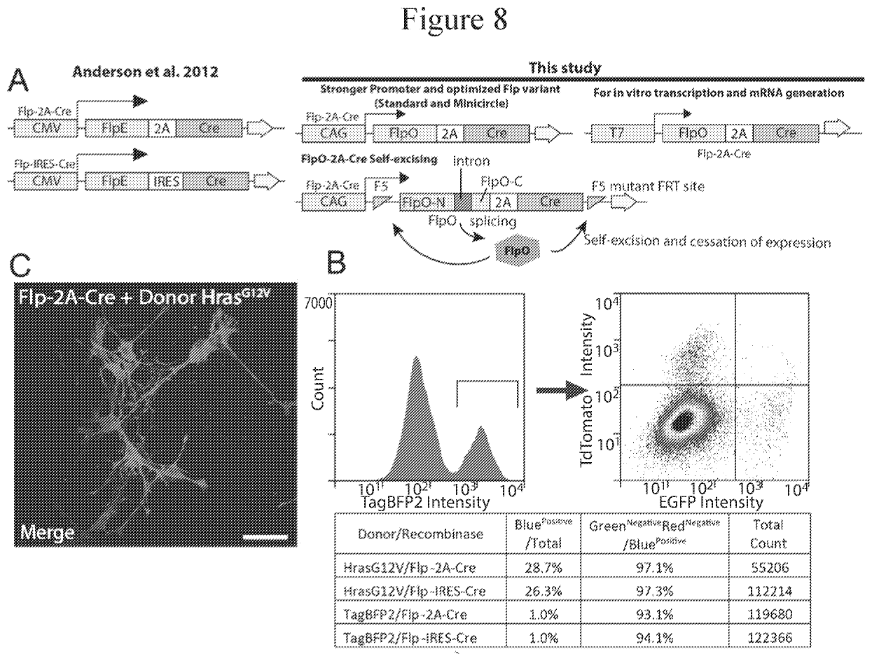

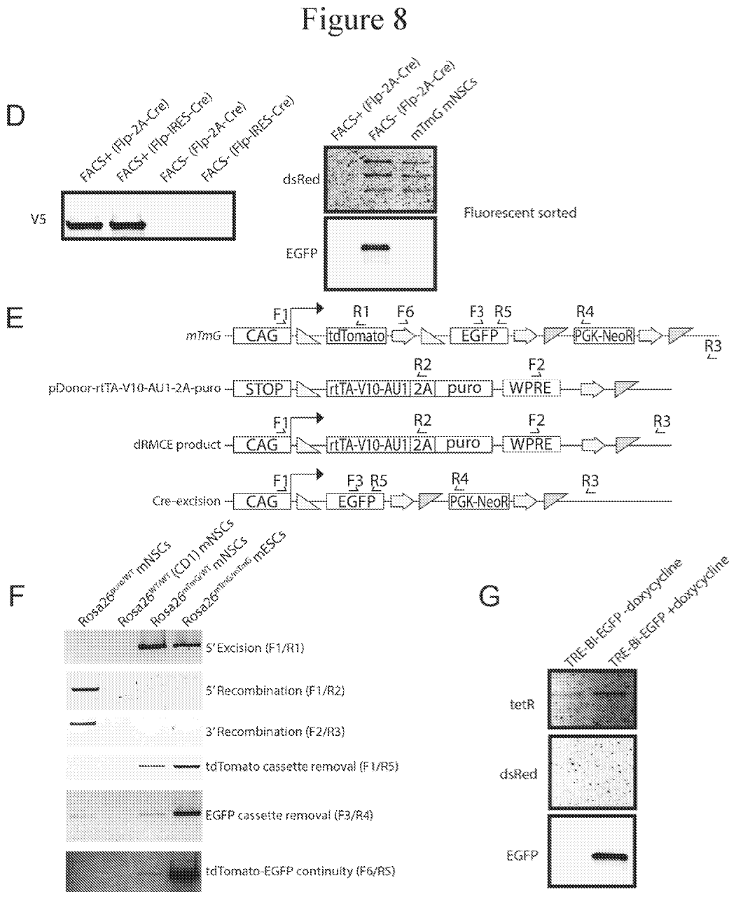

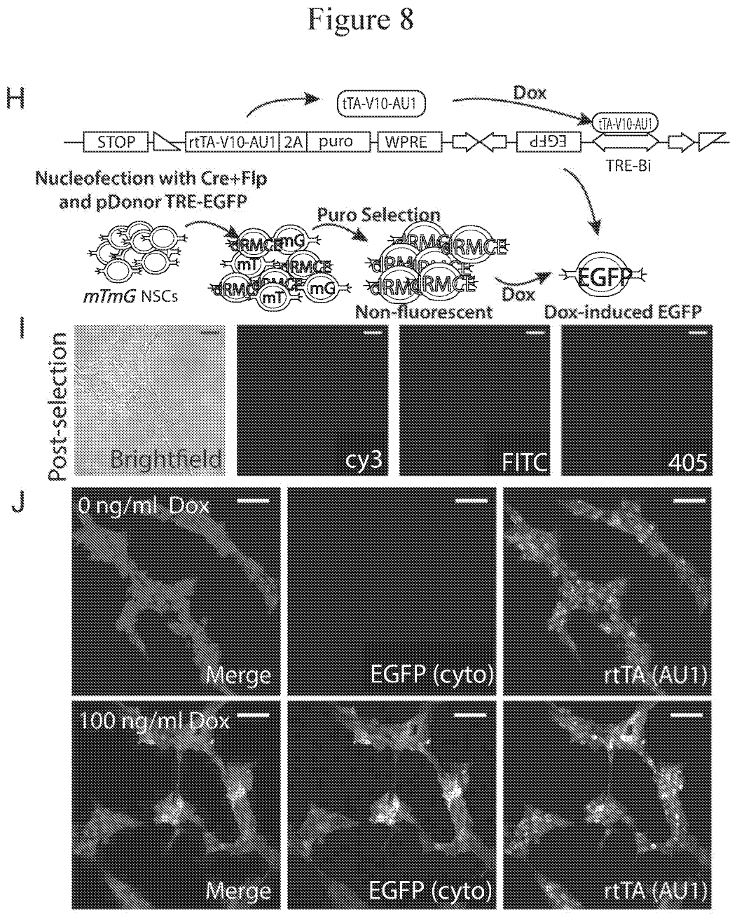

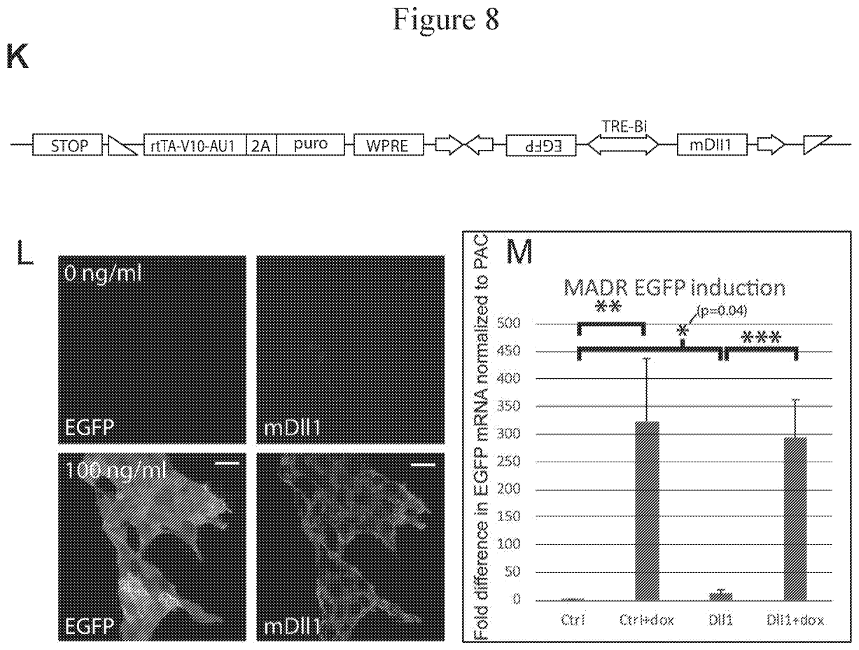

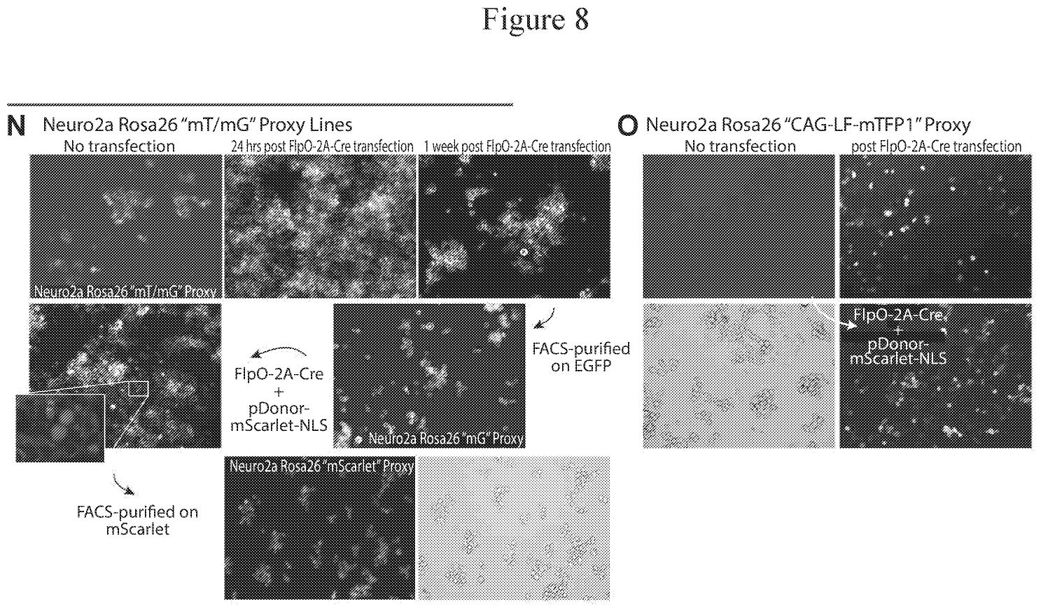

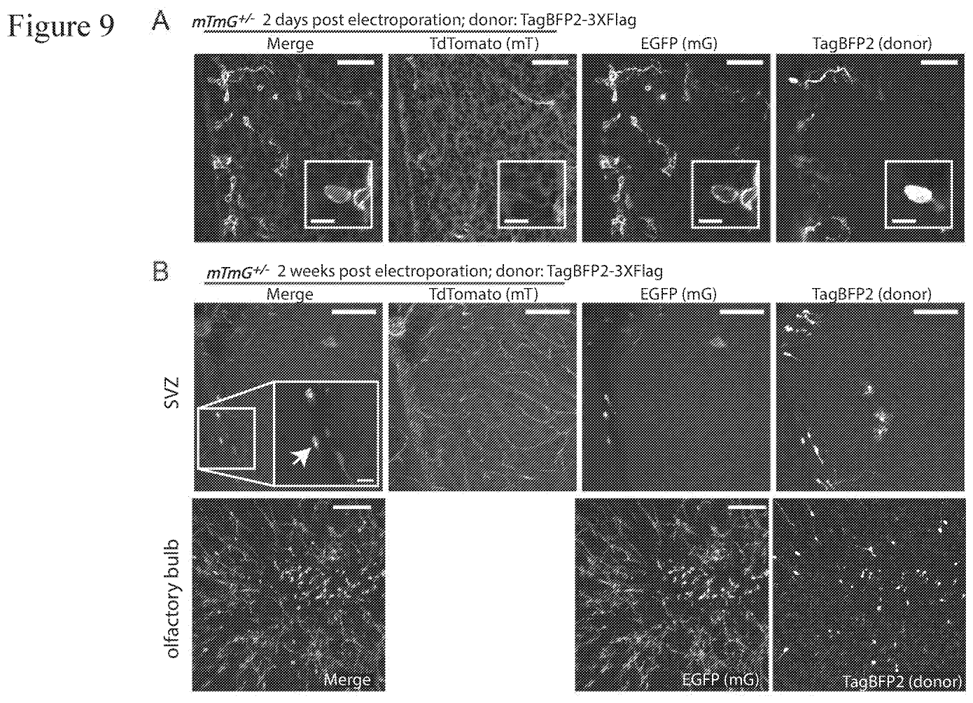

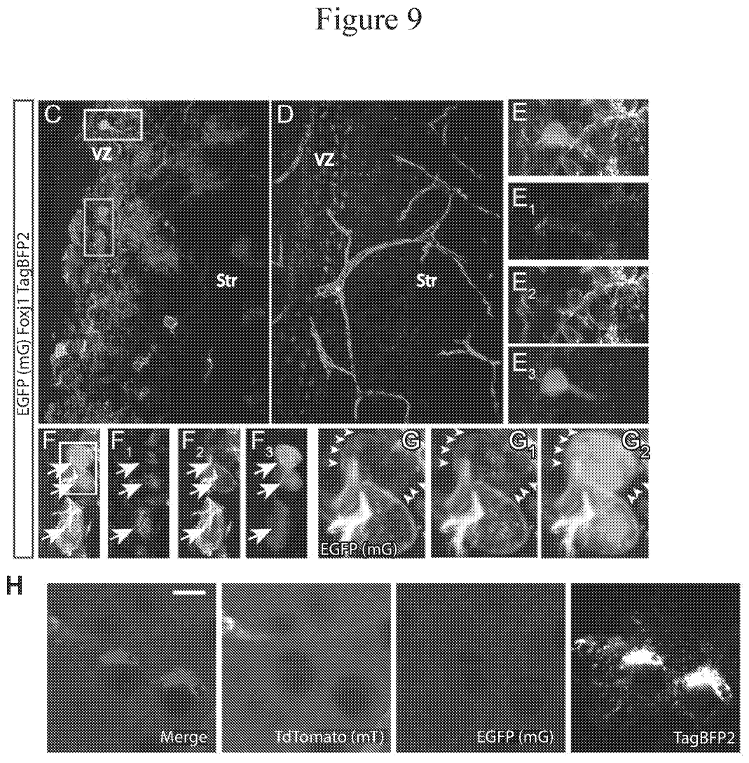

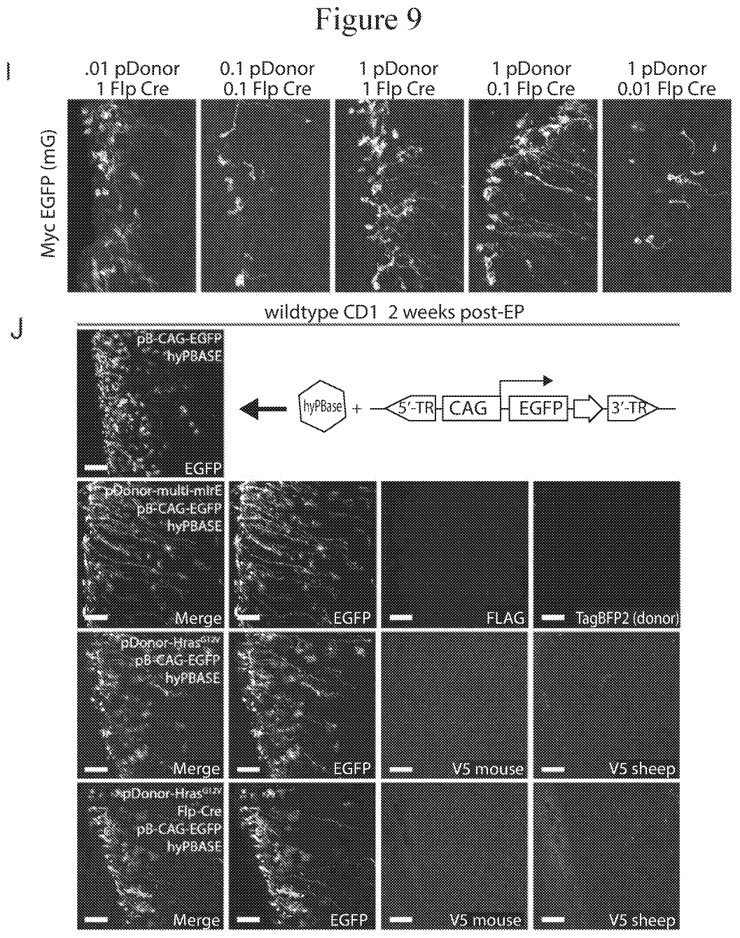

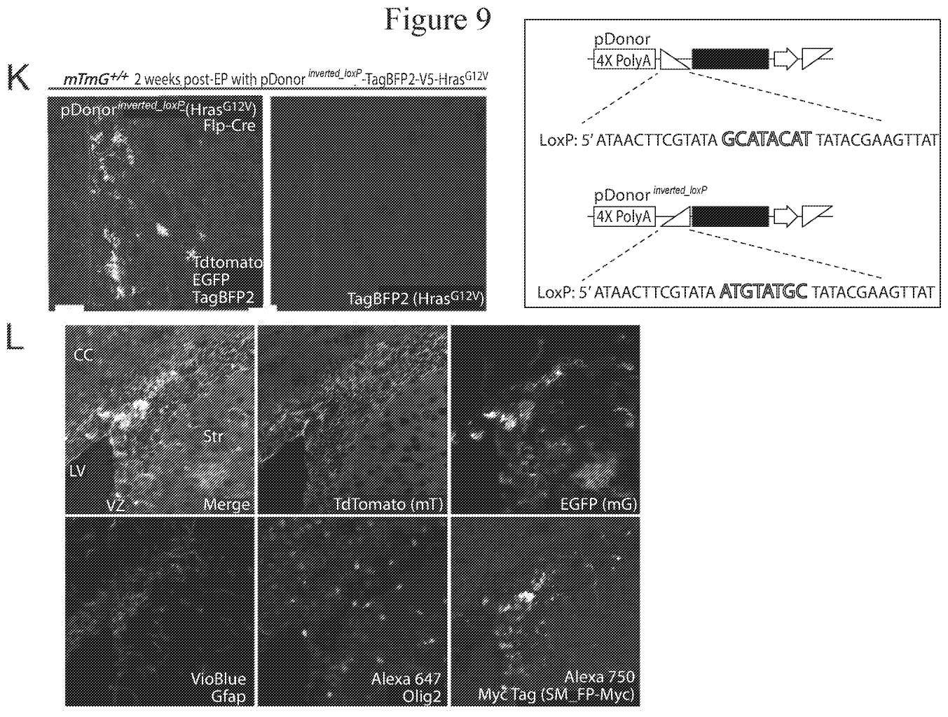

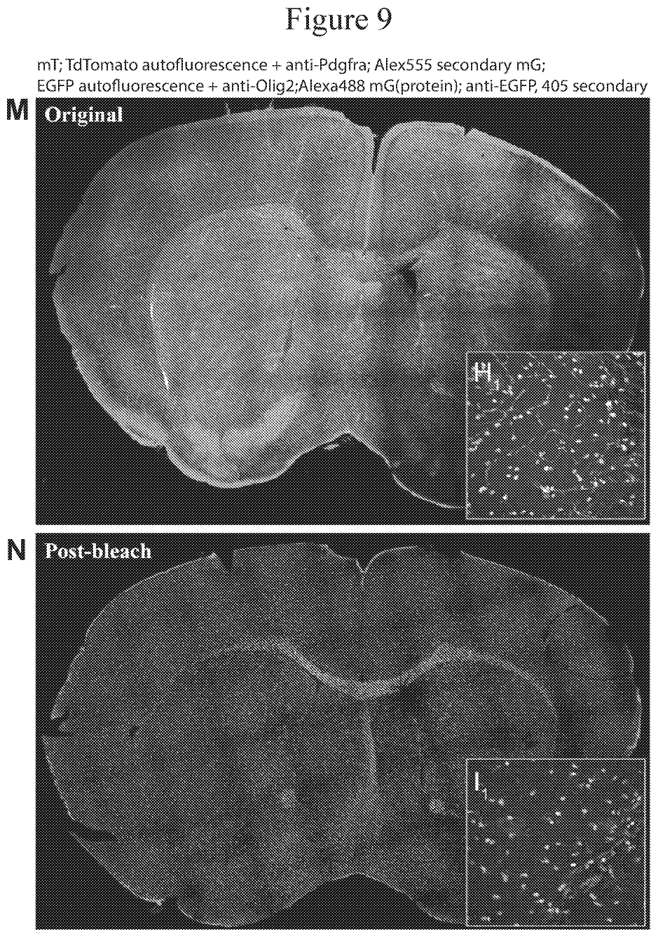

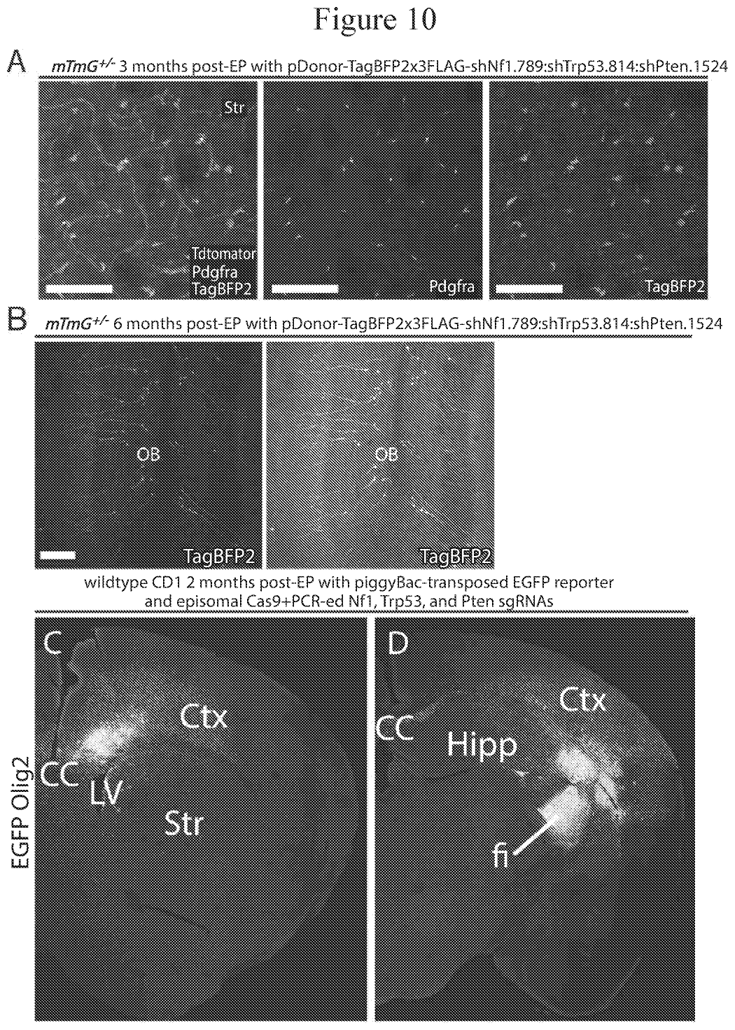

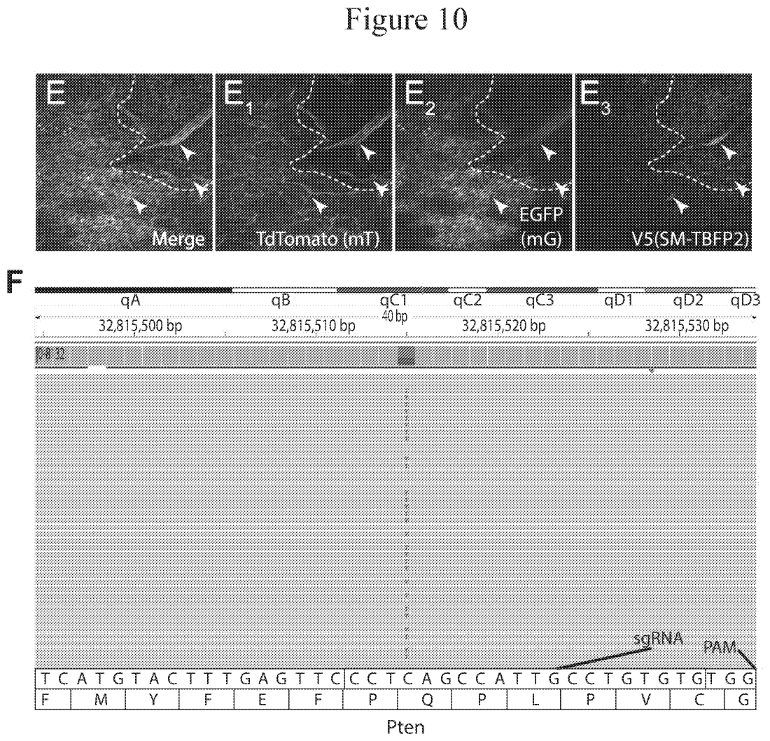

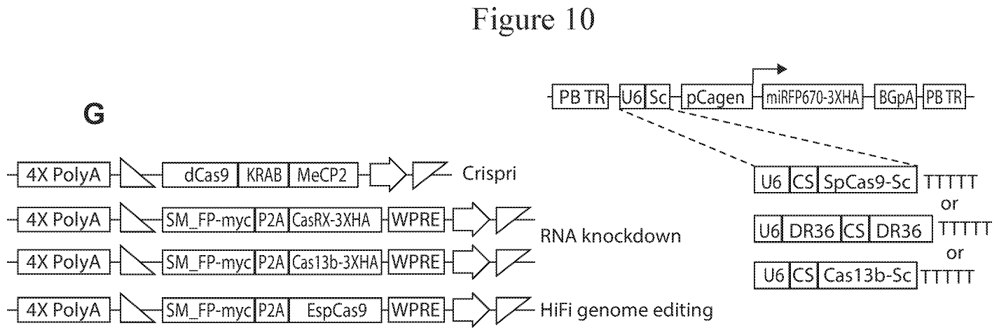

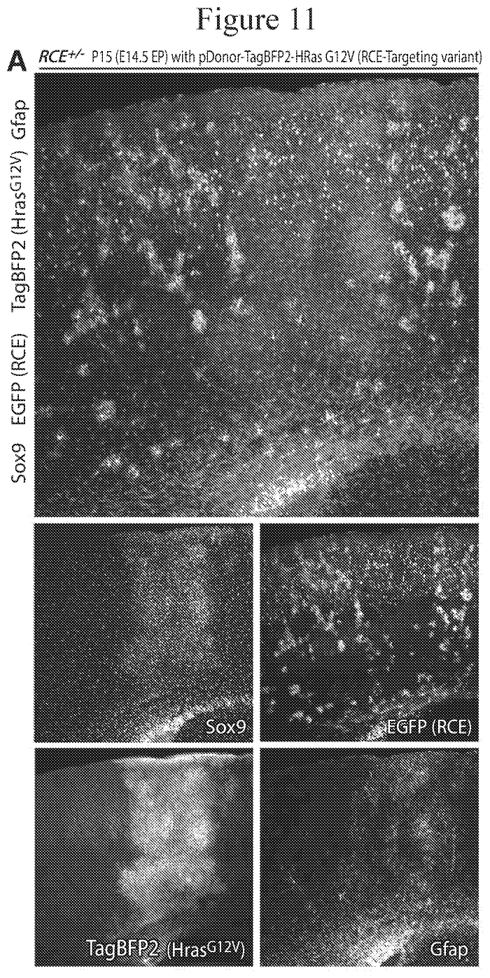

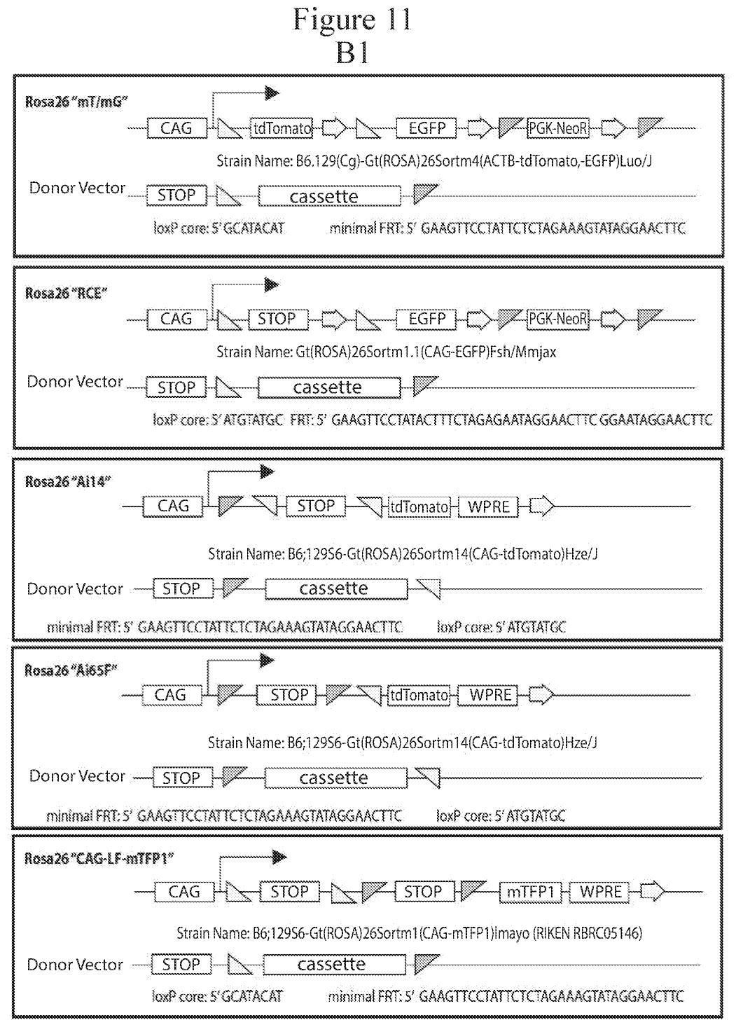

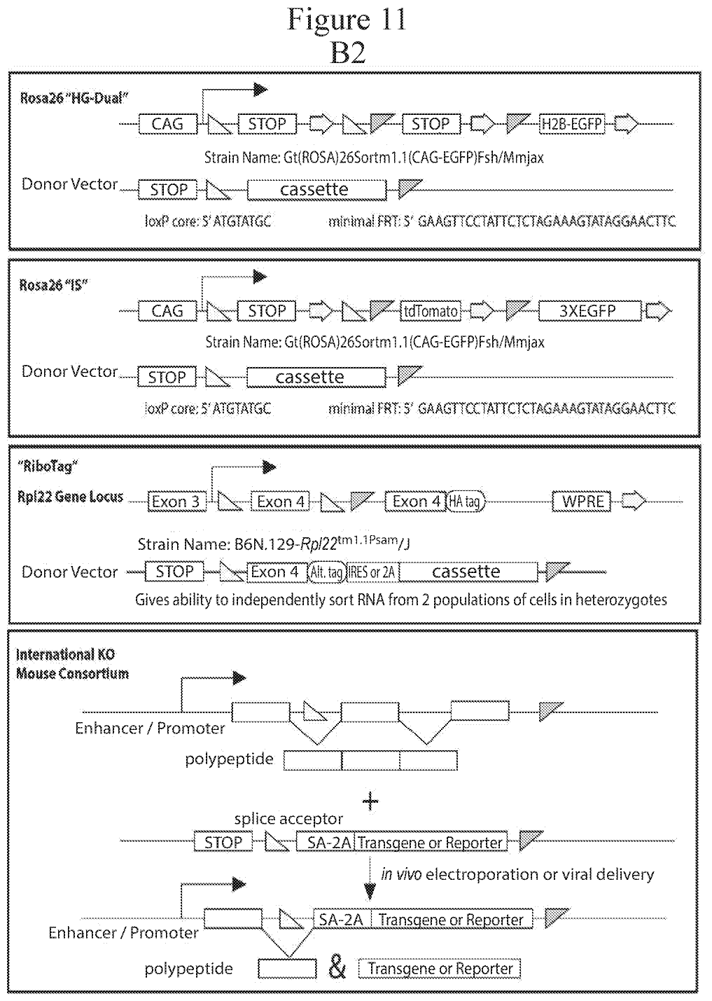

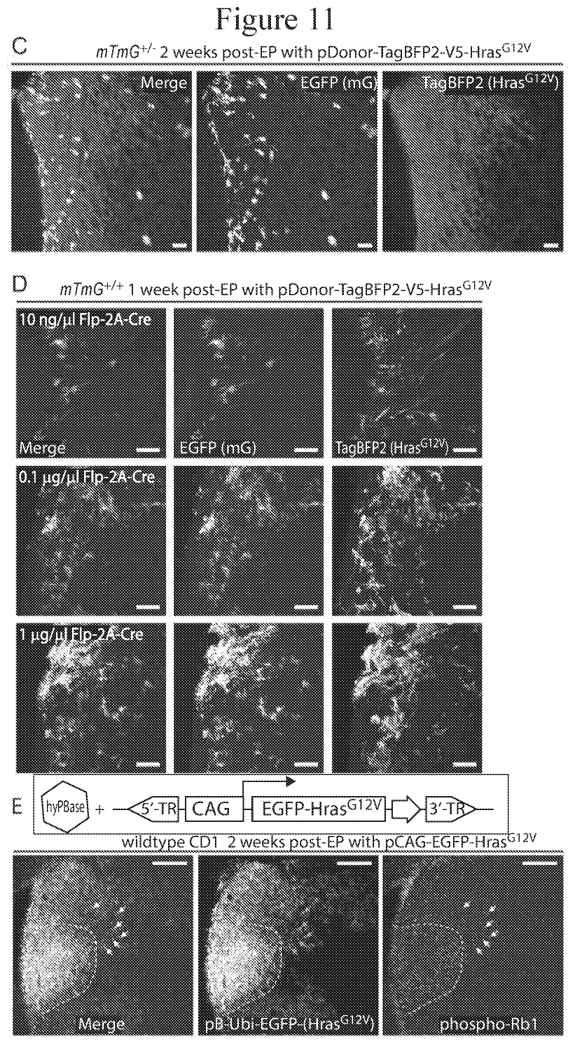

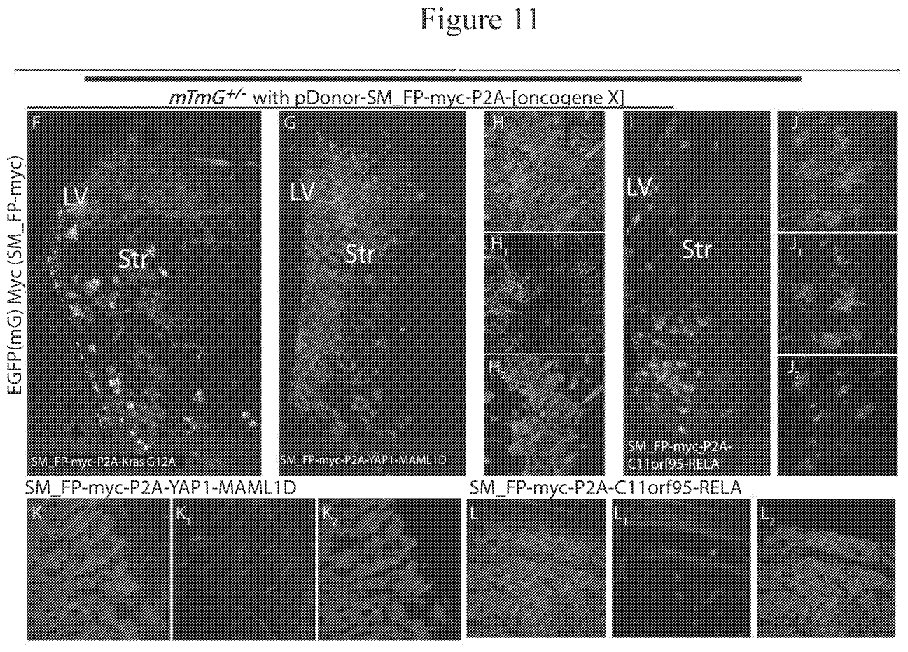

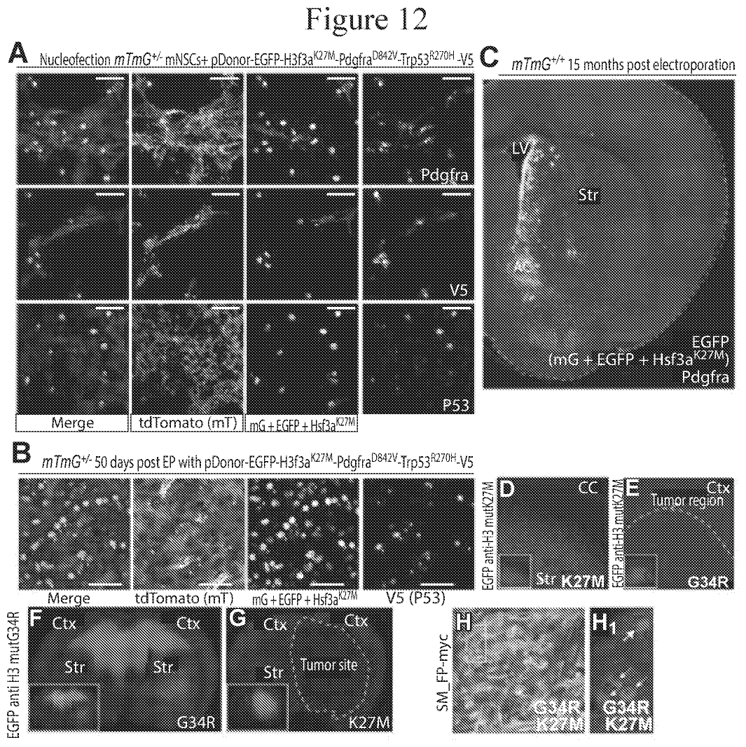

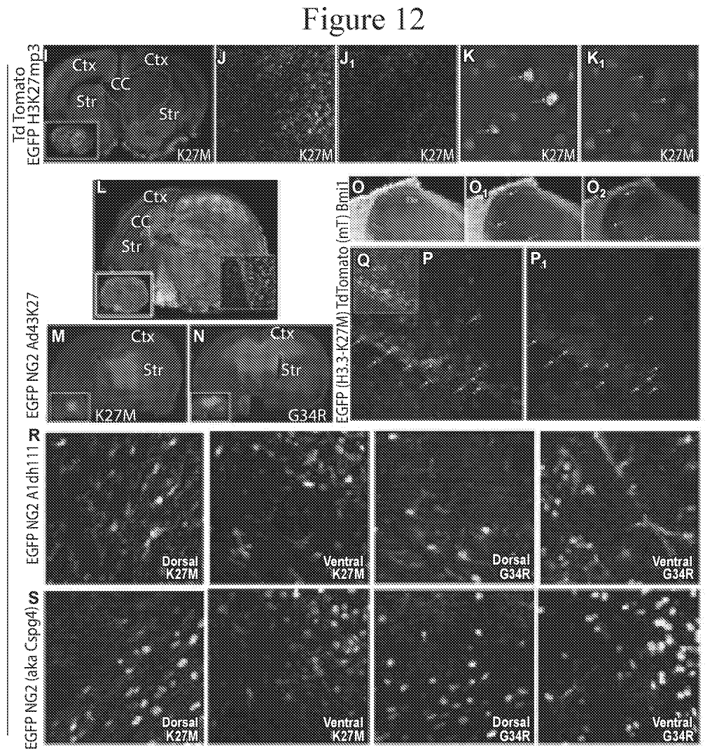

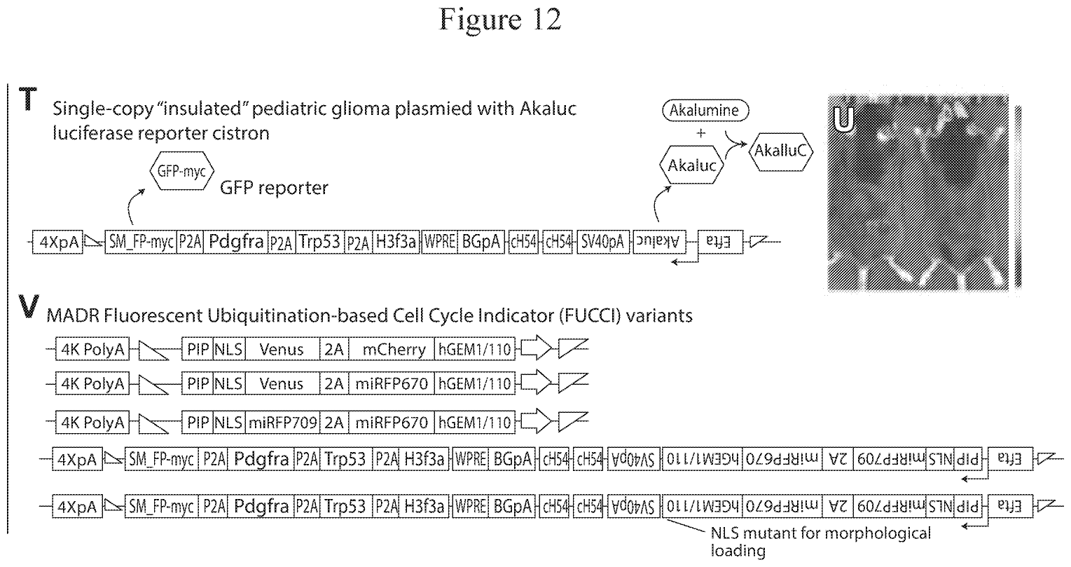

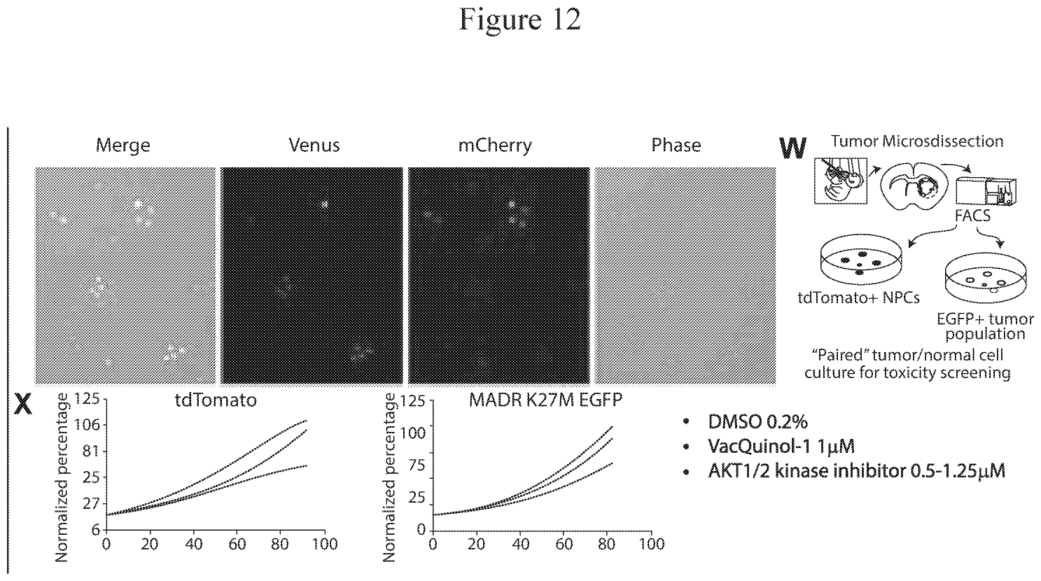

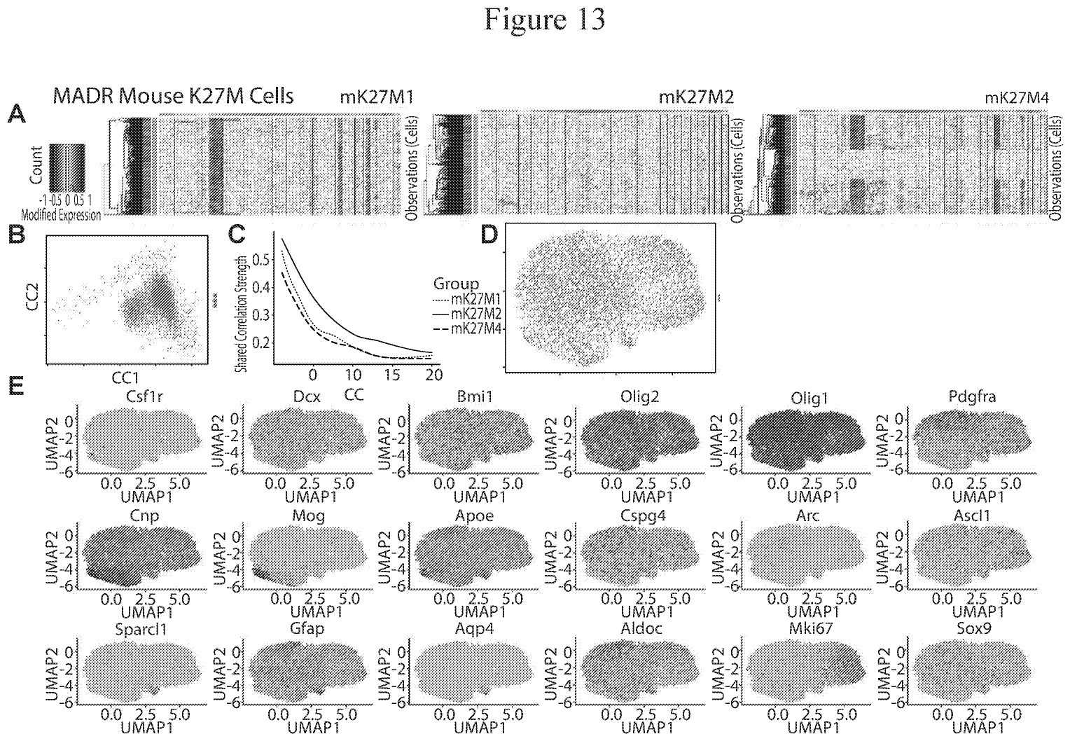

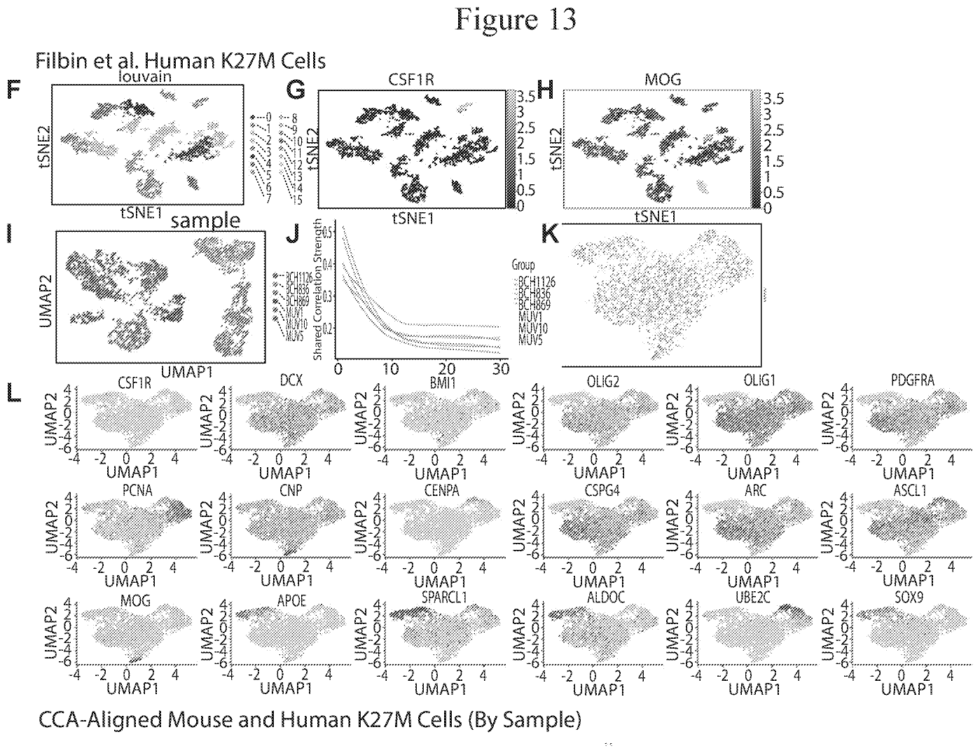

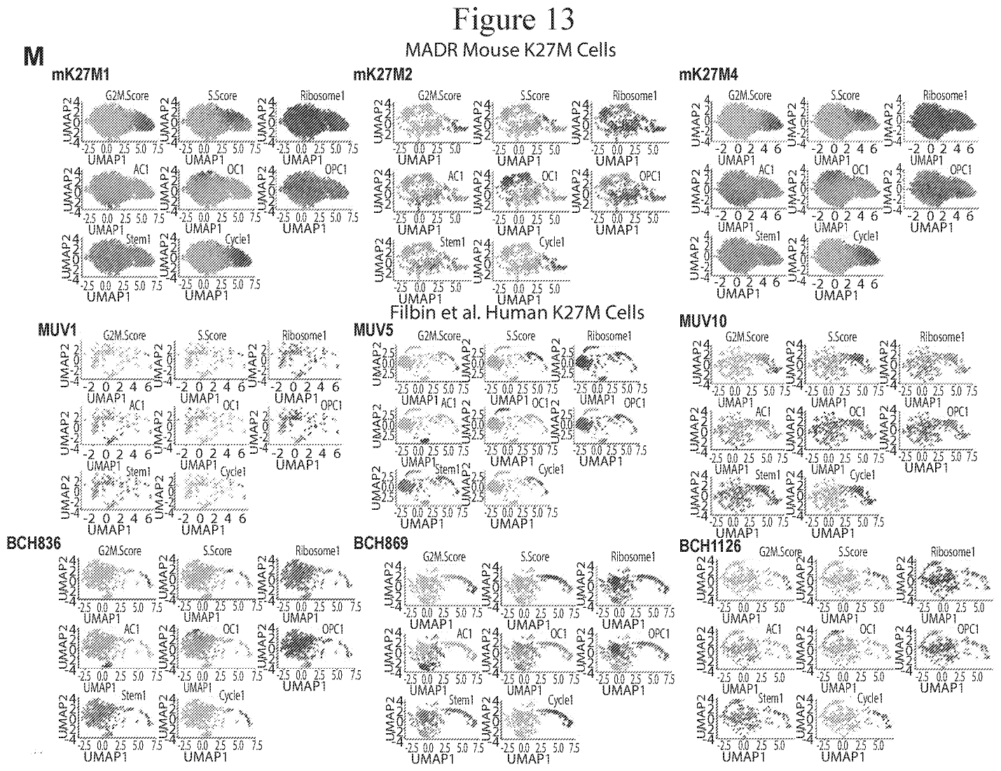

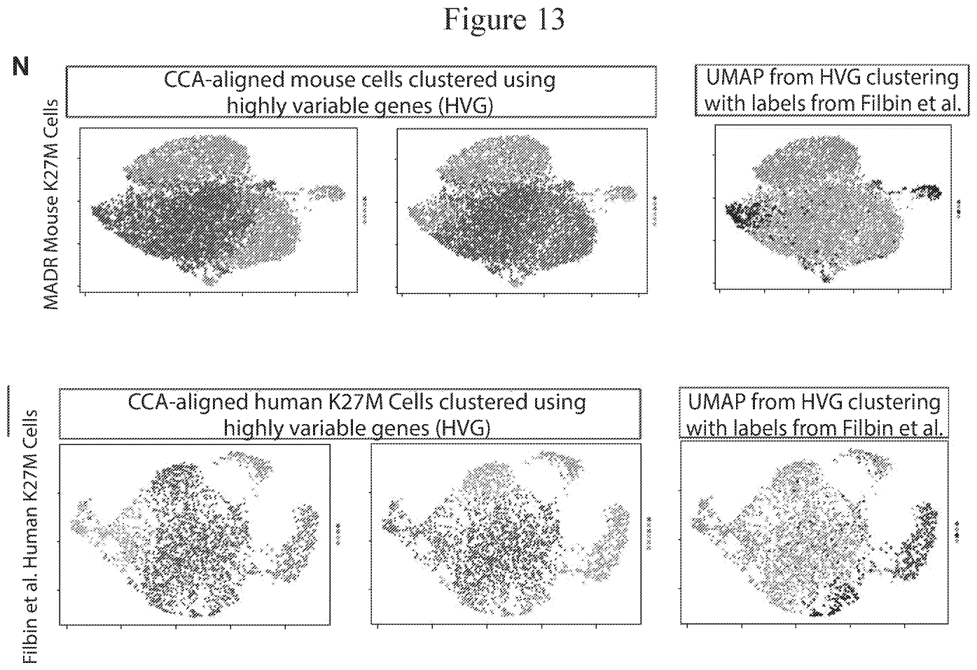

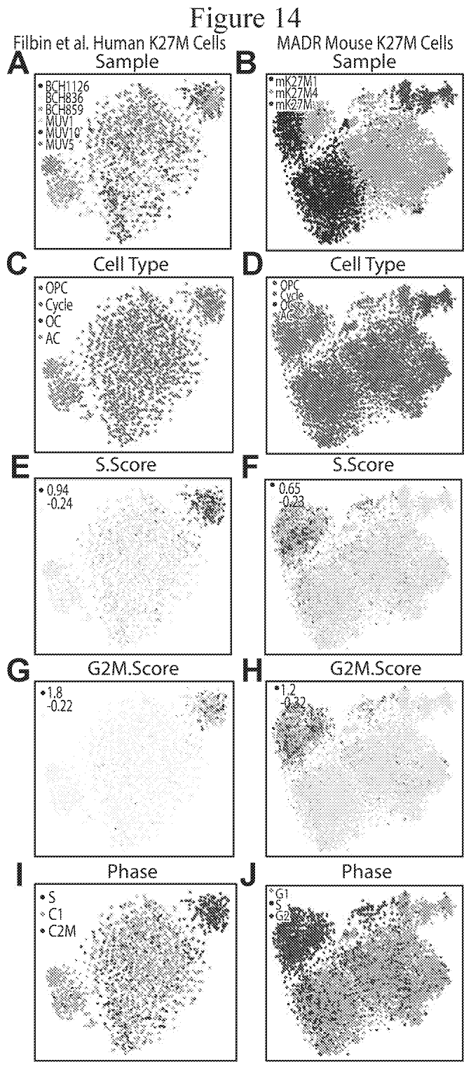

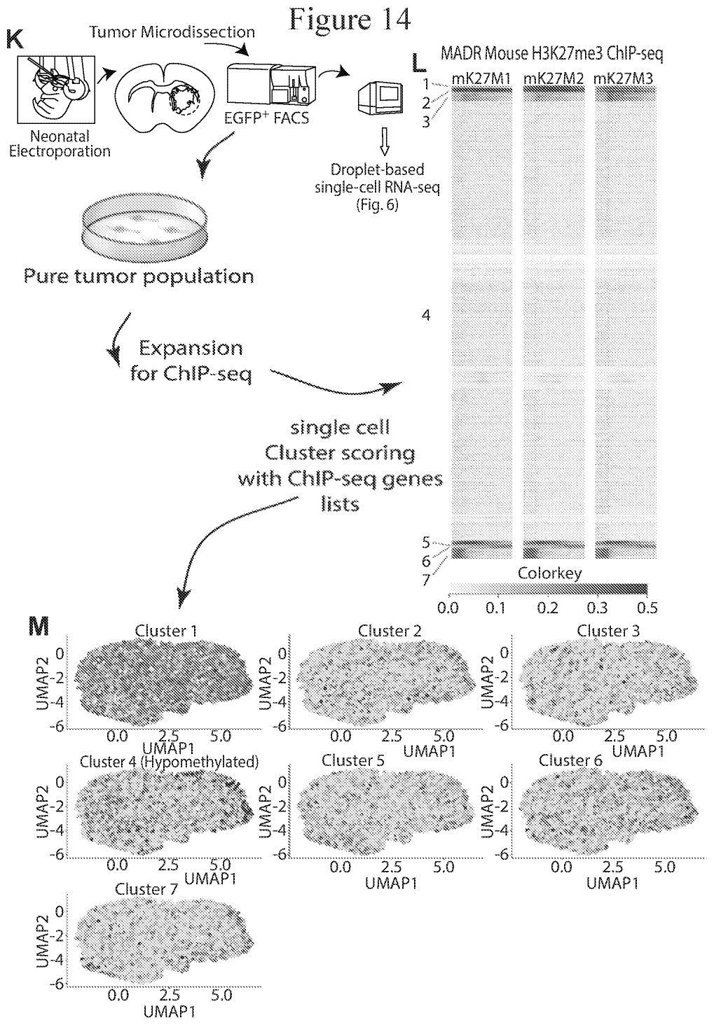

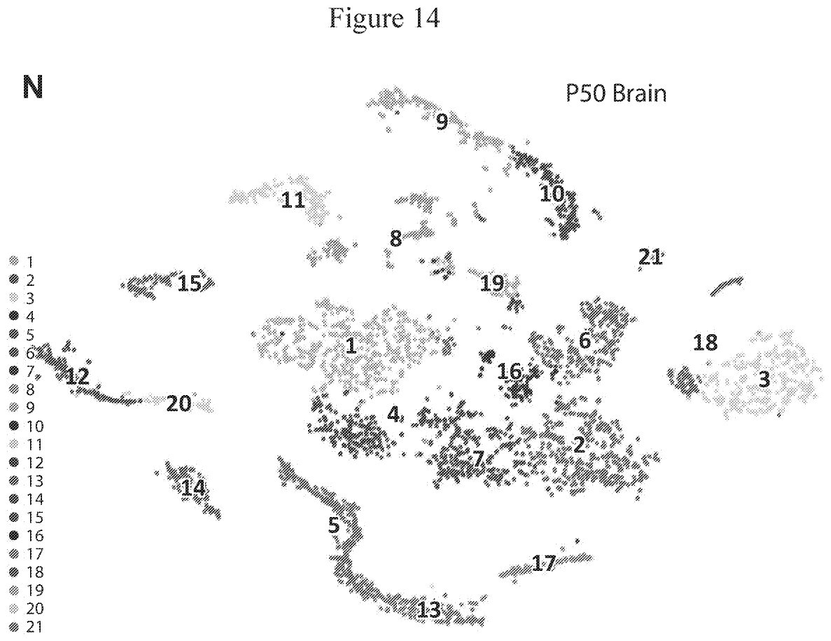

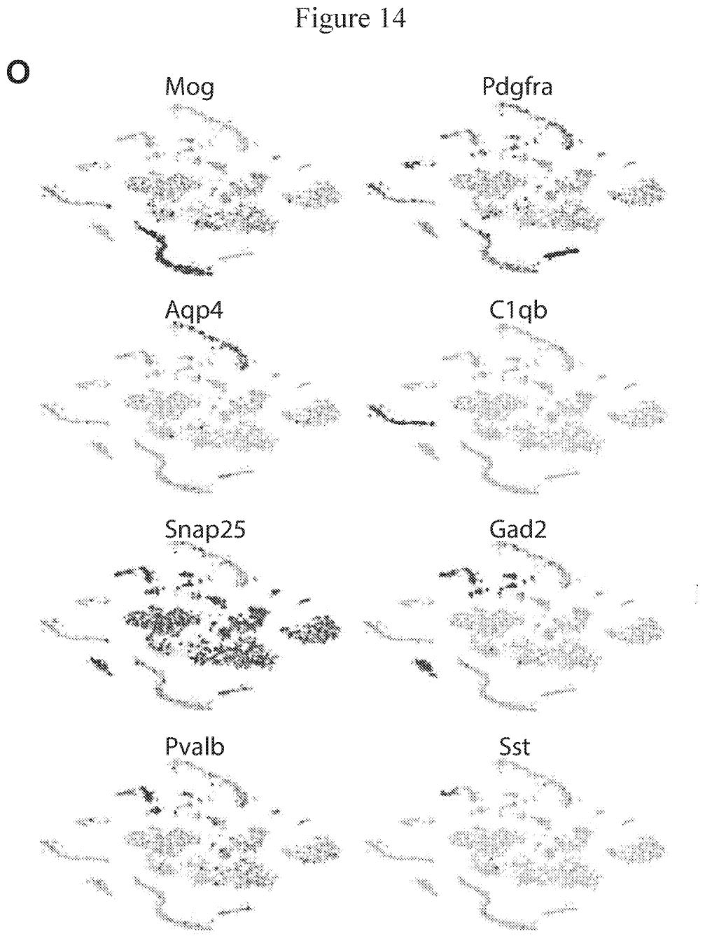

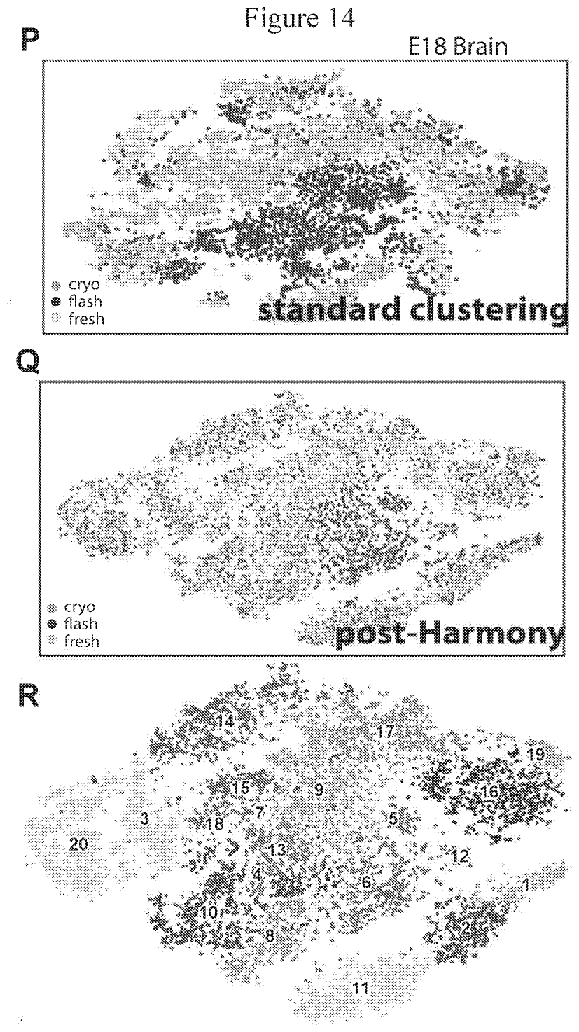

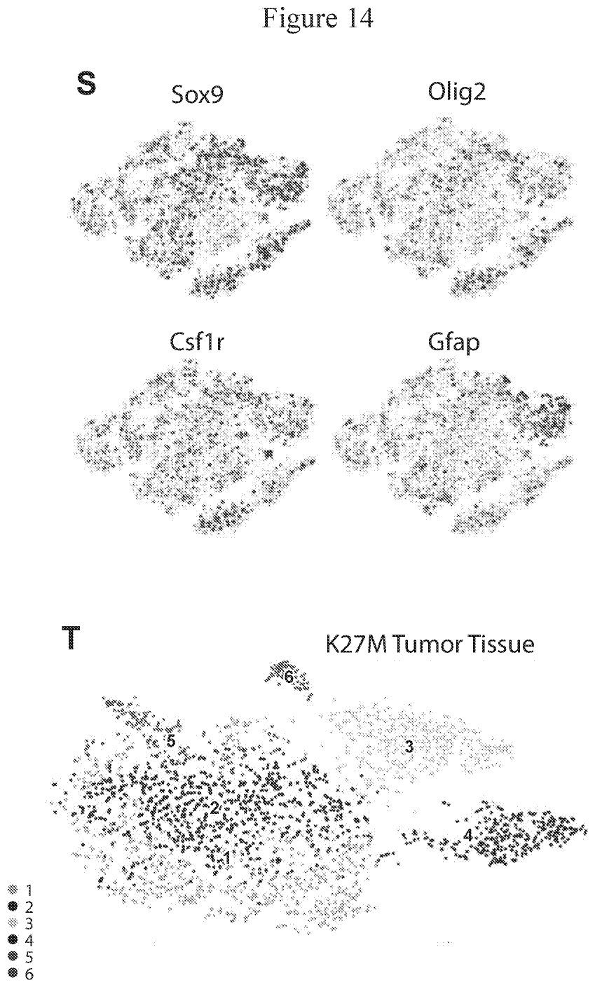

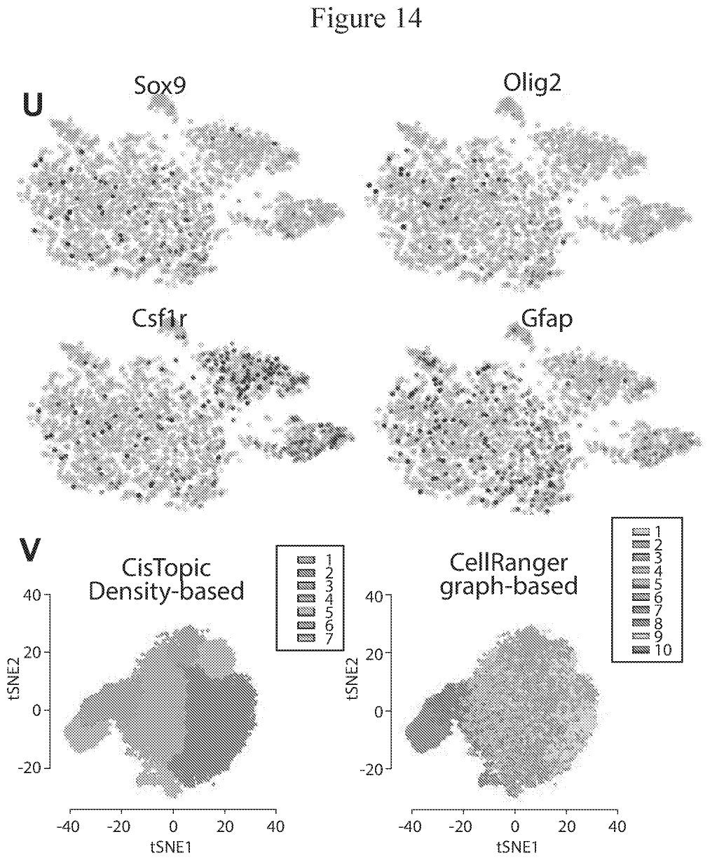

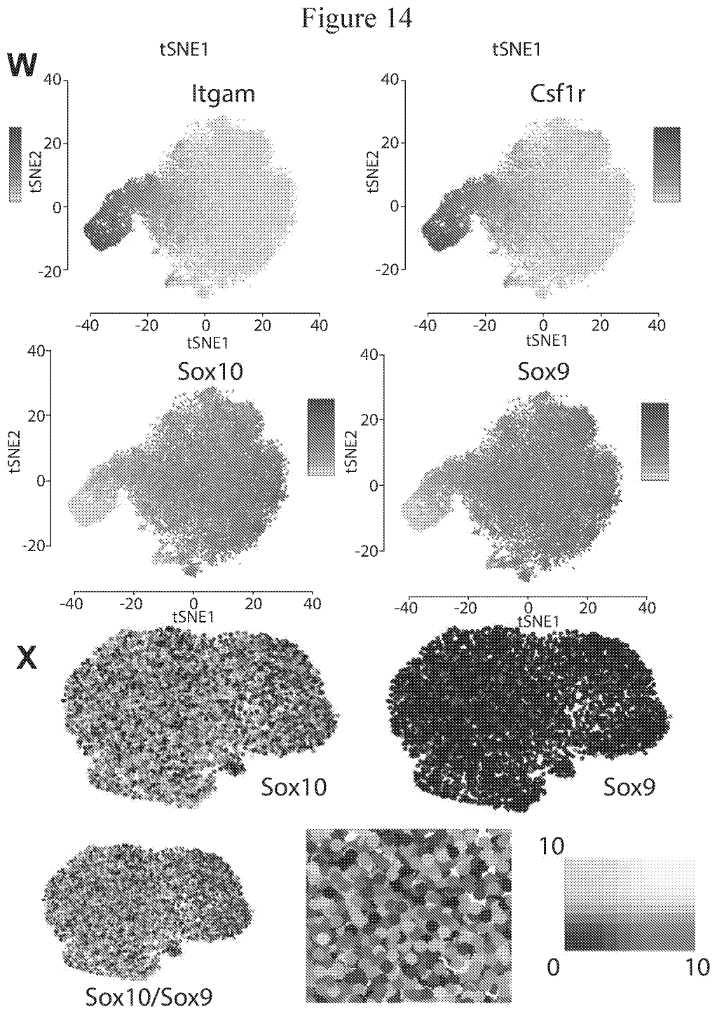

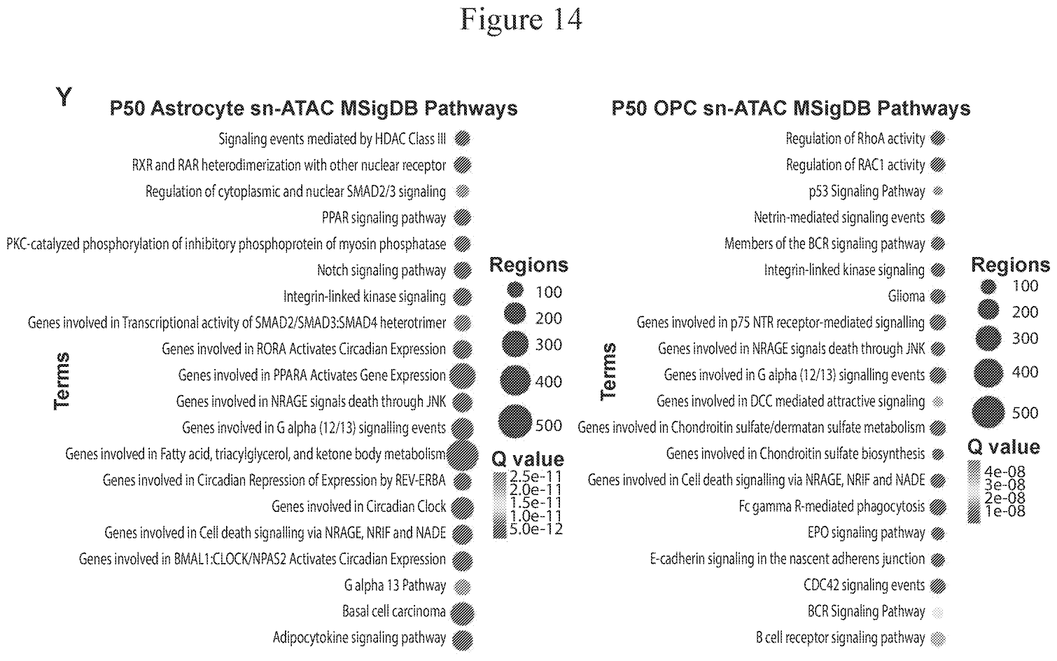

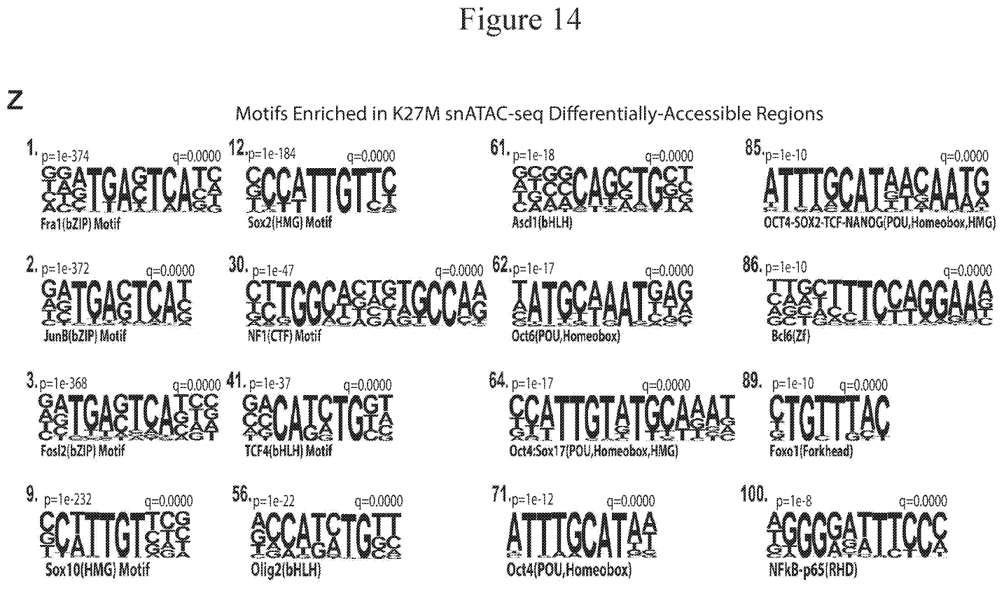

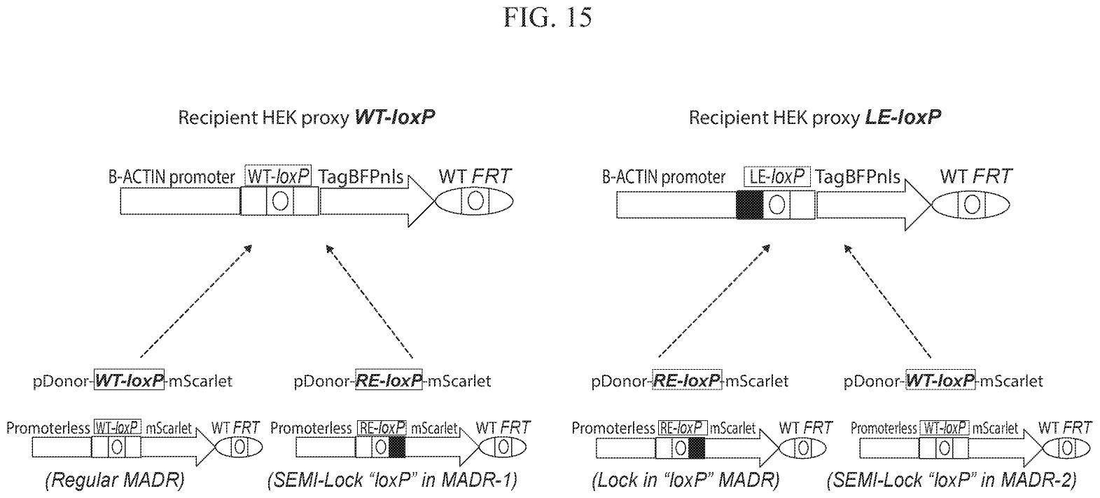

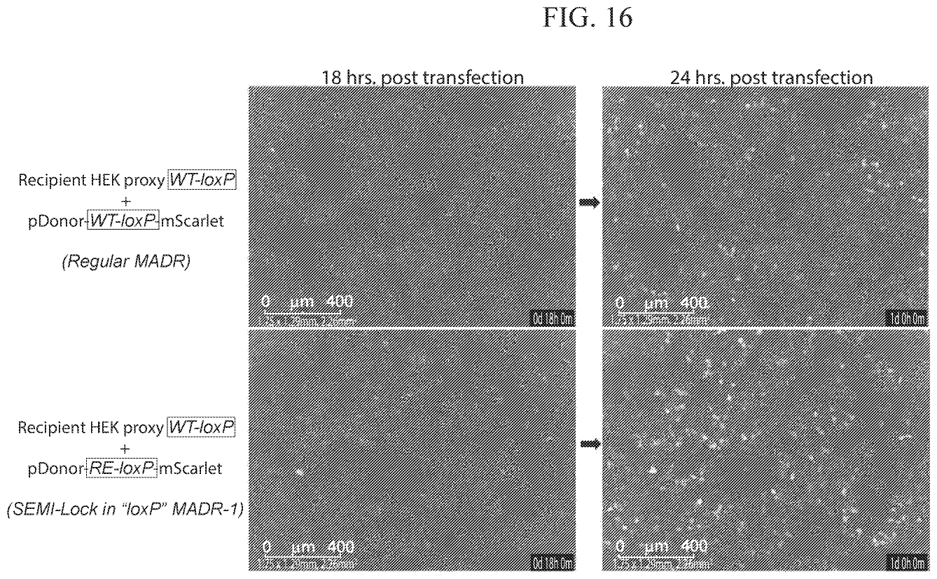

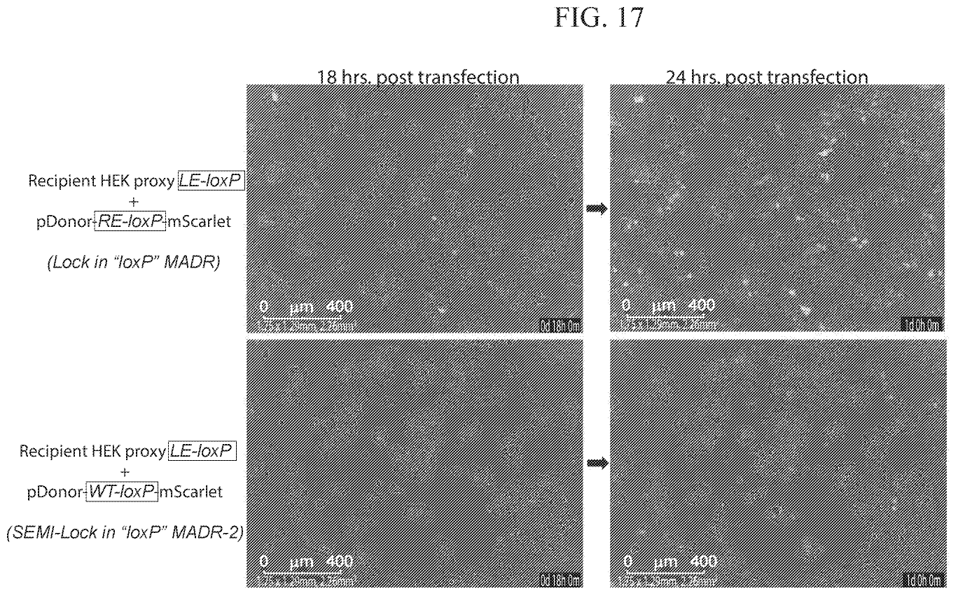

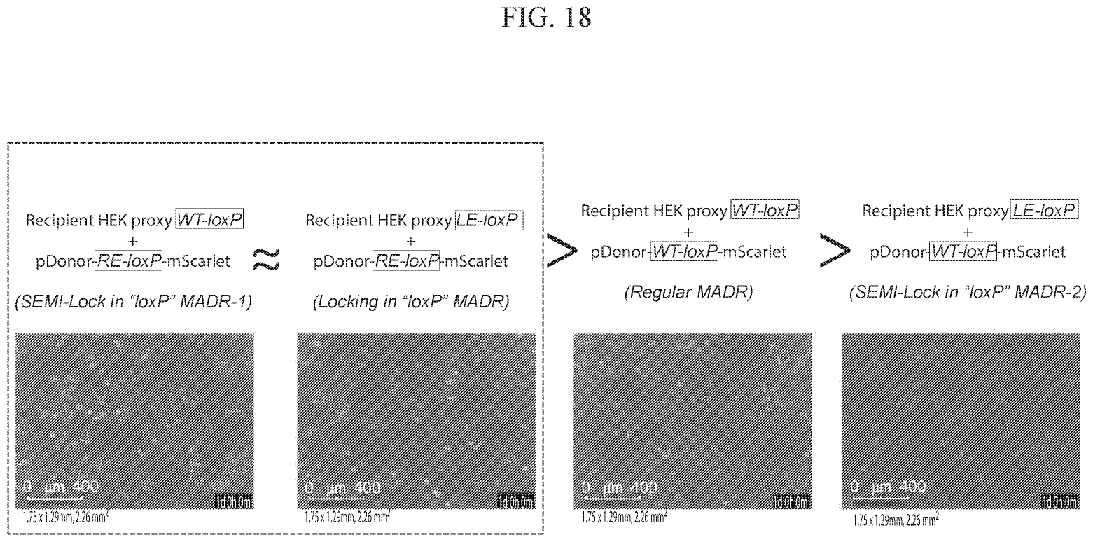

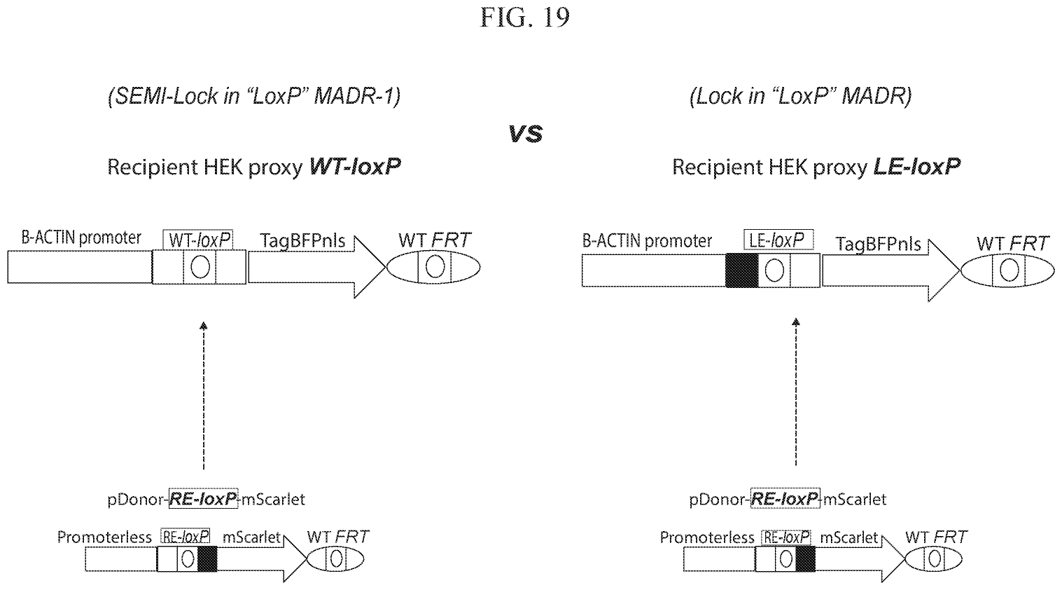

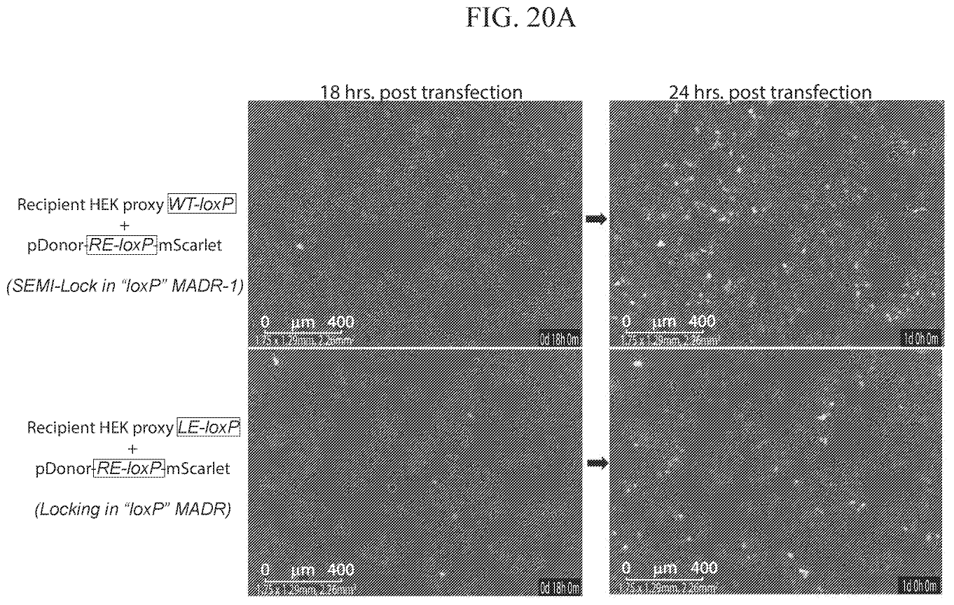

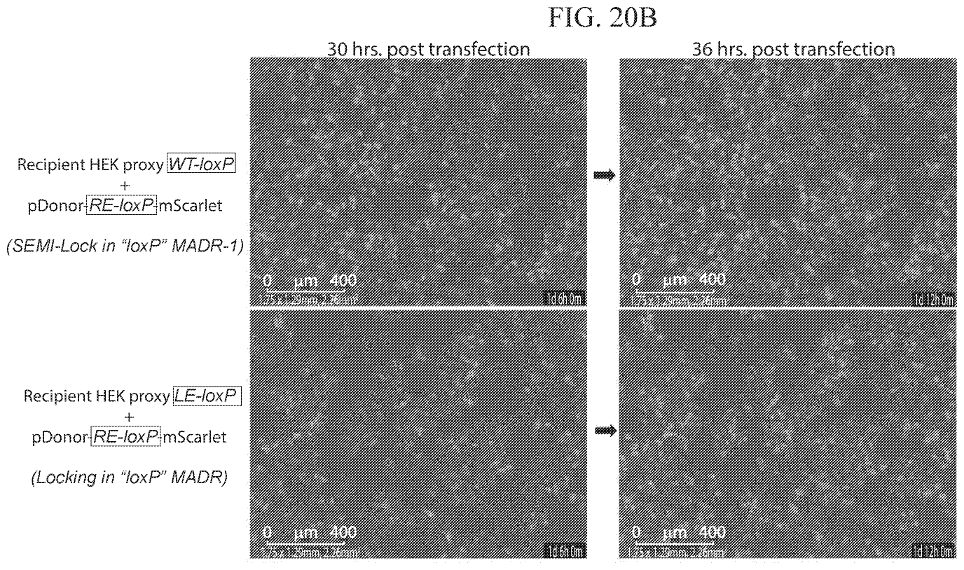

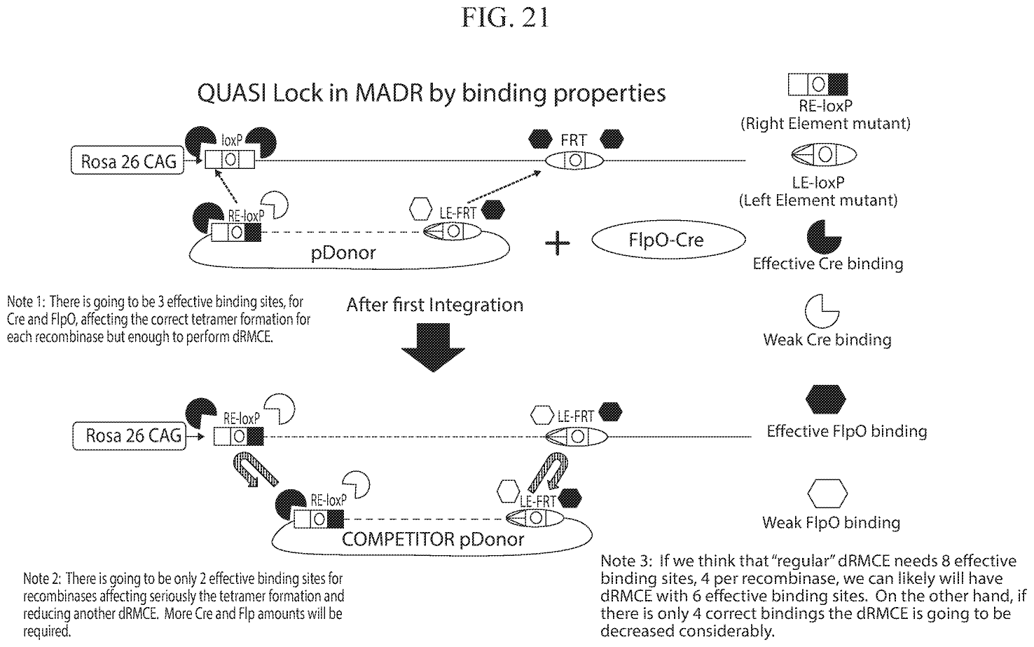

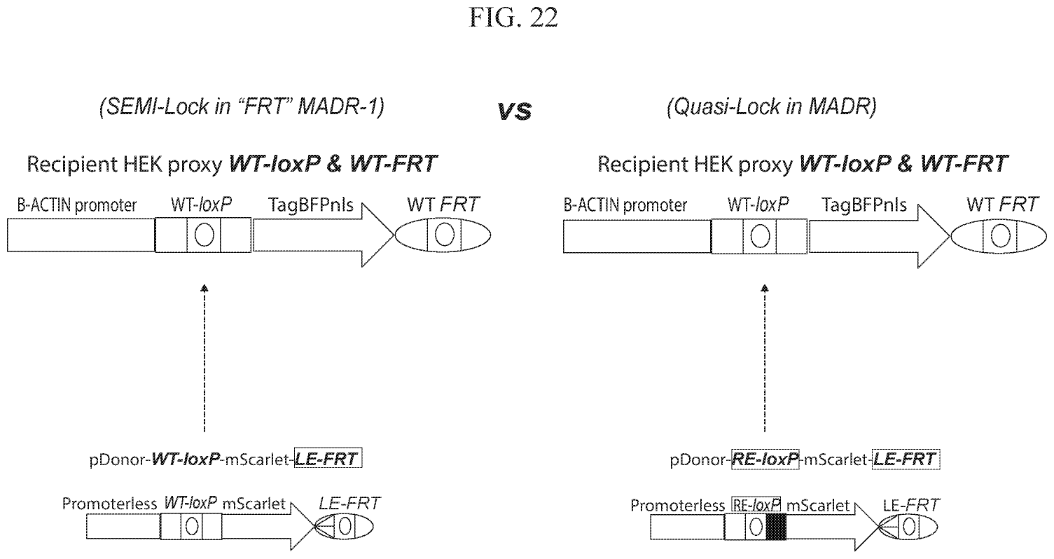

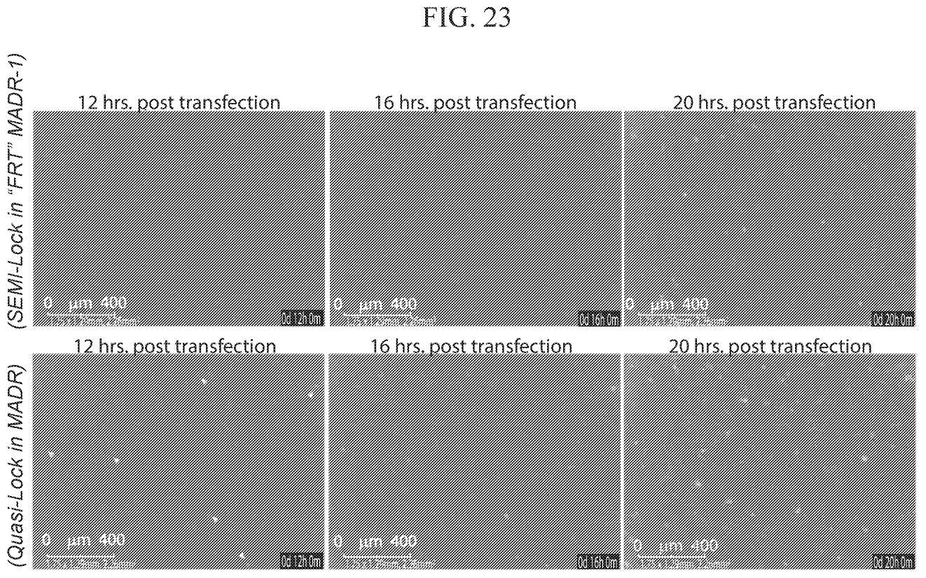

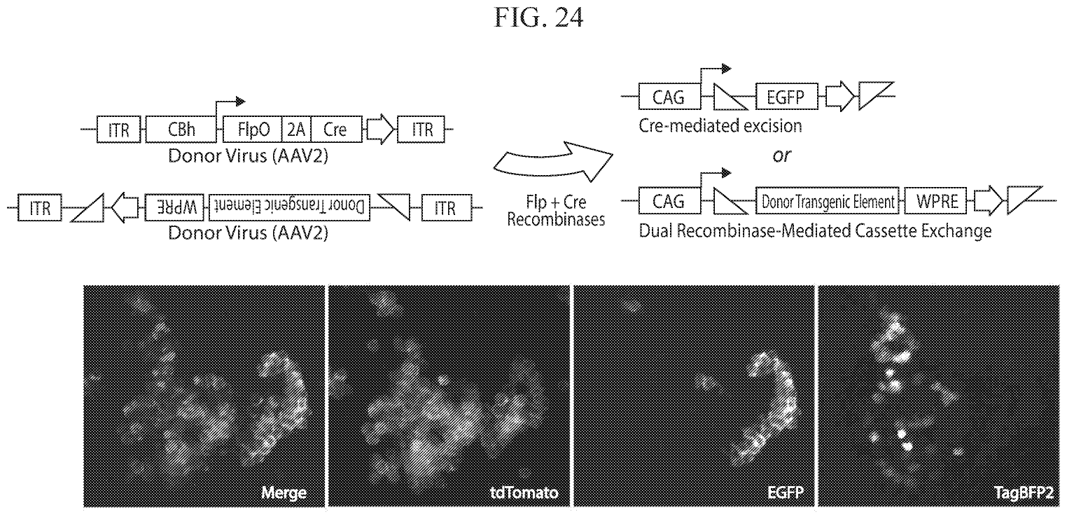

We modified loxP and FRT sites in both recipient genome and MADR pDonors. The function of MADR was validated in HEK293T cells (FIG. 15-27) and thus, we are able to exchange pDonor transgene elements using modified loxP and FRT sites.

### Example 6

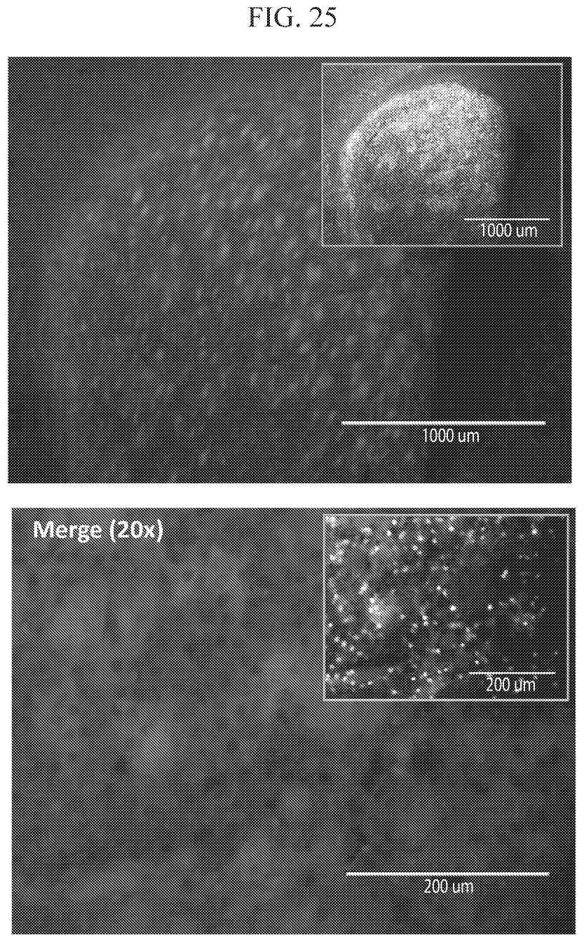

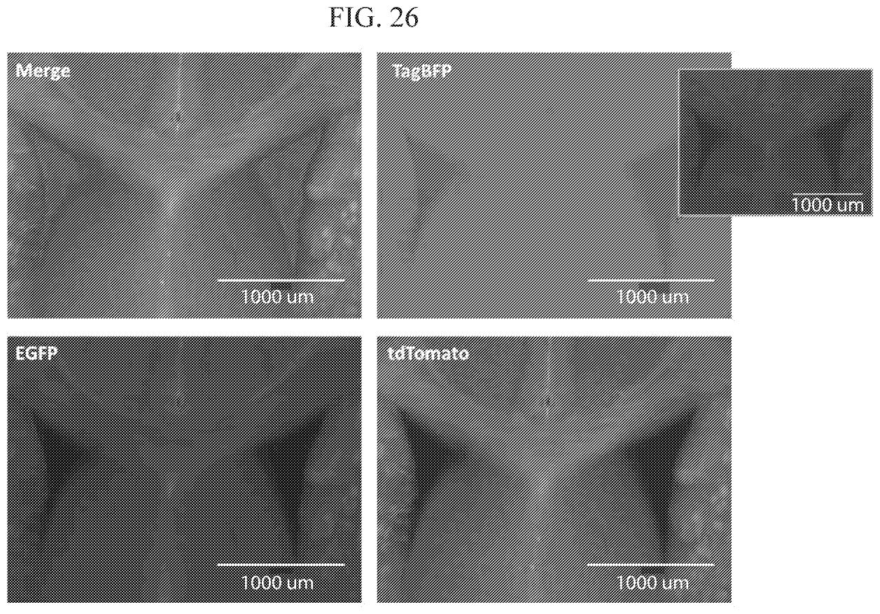

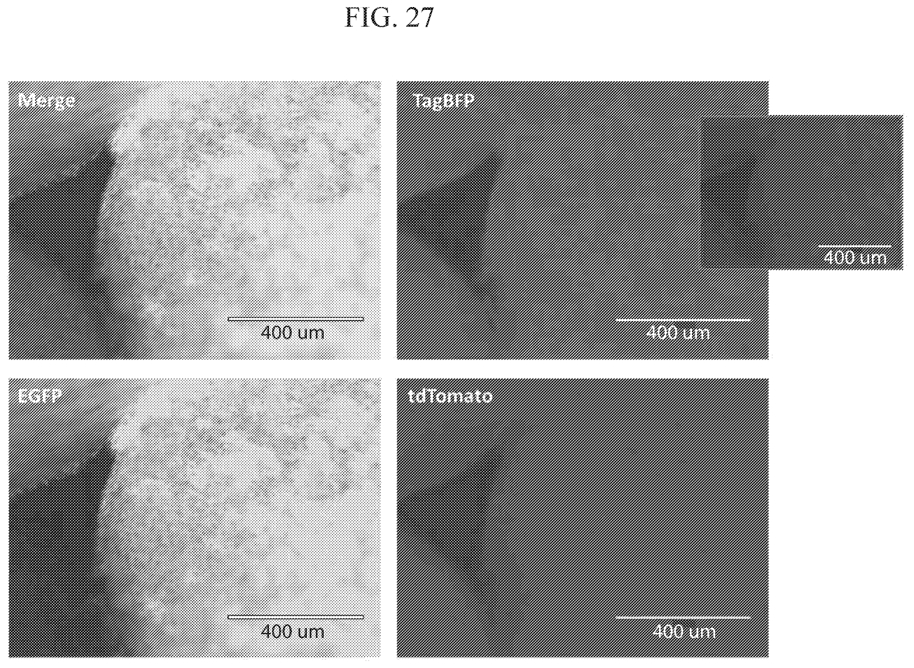

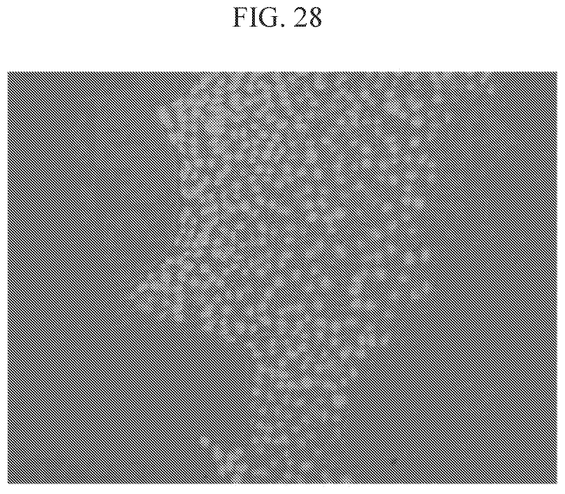

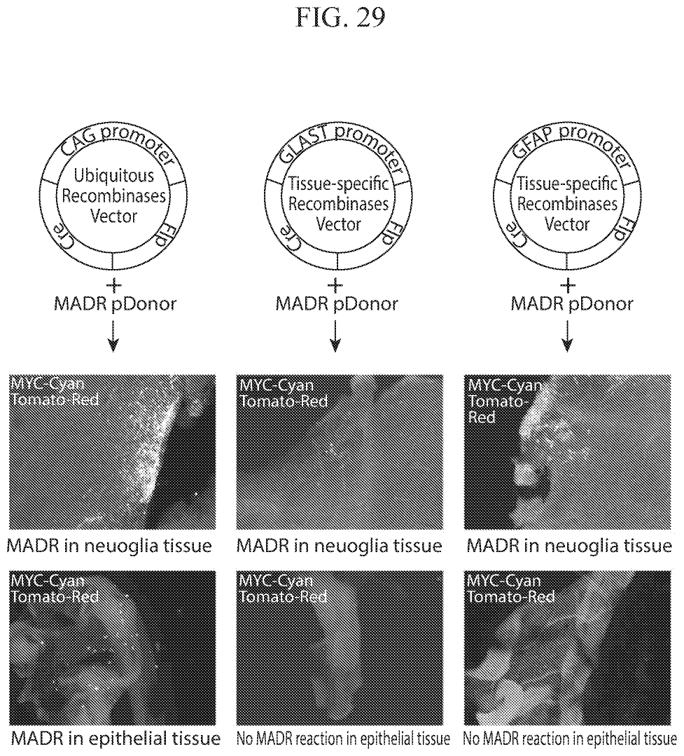

We used tissue-specific promoters on the recombinases expression vector. The function of the tissue-specific recombinases vector was validated in vivo in the mouse brain (FIG. 28), and thus, we are able to direct MADR to specific tissues.

Sequences Disclosed herein

| Seq ID | |
|---|---|
| No | Sequence |
| 1 | atgaagttatgggatgtcgtggctgtctgc |
| | ctggtgctgctccacaccgcgtccgccttc |
| | ccgctgcccgccggtaagaggcctcccgag |
| | gcgcccgccgaagaccgctccctcggccgc |
| | cgccgcgcgcccttcgcgctgagcagtgac |
| | tcaaatatgccagaggattatcctgatcag |
| | ttcgatgatgtcatggattttattcaagcc |
| | accattaaaagactgaaaaggtcaccagat |
| | aaacaaatggcagtgcttcctagaagagag |
| | cggaatcggcaggctgcagctgccaaccca |
| | gagaattccagaggaaaaggtcggagaggc |
| | cagaggggcaaaaaccggggttgtgtctta |
| | actgcaatacatttaaatgtcactgacttg |
| | ggtctgggctatgaaaccaaggaggaactg |
| | atttttaggtactgcagcggctcttgcgat |
| | gcagctgagacaacgtacgacaaaatattg |
| | aaaaacttatccagaaatagaaggctggtg |
| | agtgacaaagtagggcaggcatgttgcaga |
| | cccatcgcctttgatgatgacctgtcgttt |
| | ttagatgataacctggtttaccatattcta |
| | agaaagcattccgctaaaaggtgtggatgt |
| | atctga |
| 2 | MKLWDVVAVCLVLLHTASAFPLPAGKRPPE |
| | APAEDRSLGRRRAPFALSSDSNMPEDYPDQ |
| | FDDVMDFIQATIKRLKRSPDKQMAVLPRRE |
| | RNRQAAAANPENSRGKGRRGQRGKNRGCVL |
| | TAIHLNVTDLGLGYETKEELIFRYCSGSCD |
| | AAETTYDKILKNLSRNRRLVSDKVGQACCR |
| | PIAFDDDLSFLDDNLVYlflLRKHSAKRCG |
| | CI |

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A system, comprising:
a promoter-less donor vector, comprising:
a polyadenylation signal or transcription stop element upstream from a transgene or a nucleic acid encoding an RNA,
the transgene or nucleic acid encoding an RNA, and
paired recombinase recognition sites; and
one expression vector, comprising two genes encoding recombinases specific to the paired recombinase recognition sites, or
two expression vectors, the first expression vector comprising one gene encoding a first recombinase that is specific to one of the paired recombinase recognition sites, and the second expression vector comprising one gene encoding a second recombinase that is specific to the other of the paired recombinase recognition sites.

2. The system of claim 1,
wherein the promoter-less donor vector is selected from the group consisting of plasmid, viral vector, and bacterial artificial chromosome (BAC), or
wherein the promoter-less donor vector comprises at least four polyadenylation signals upstream from the transgene or nucleic acid encoding the RNA, or
wherein the promoter-less donor vector further comprises a post-transcriptional regulatory element, or
wherein the promoter-less donor vector further comprises a polyadenylation signal downstream from the transgene or nucleic acid encoding the RNA, or
wherein the promoter-less donor vector further comprises an open reading frame (ORF) that begins with a splice acceptor, or wherein the promoter-less donor vector further comprises a fluorescent reporter, or
a combination thereof.

3-7. (canceled)

8. The system of claim 2, wherein the viral vector is an adeno-associated viral (AAV) vector.

9. The system of claim 1, wherein the expression vector comprising recombinases are under tissue-specific promoters.

10. The system of claim 1,
wherein the paired recombinase recognition sites are loxP and flippase recognition target (FRT), and the recombinases are cre and flp, or
wherein the paired recombinase recognition sites are modified loxP and/or modified flippase recognition target (FRT), and the recombinases are cre and flp, or
wherein the paired recombinase recognition sites are VloxP and flippase recognition target (FRT), and the recombinases are VCre and flp, or
wherein the paired recombinase recognition sites are SloxP and flippase recognition target (FRT), and the recombinases are SCre and flp, or
wherein the recombinase is PhiC31 recombinase and the recombinase recognition sites are attB and attP, or
wherein the recombinase is Nigri, Panto, or Vika and recombinase recognition sites are nox, pox, and vox, respectively, or
wherein one or both of the paired recombinase recognition sites comprise a mutation, or
a combination thereof.

11-16. (canceled)

17. The system of claim 1,
wherein the RNA is siRNA, snRNA, sgRNA, lncRNA or miRNA, or
wherein the transgene or the RNA comprises disease associated mutations, or
wherein the transgene or the RNA comprise a gain-of-function (GOF) gene mutation, loss-of-function (LOF) gene mutation, or both, or
wherein the transgene comprises a factor that prevents apoptosis or promotes survival of a neuronal cell, increases the proliferation of a neuronal cell, or promotes differentiation of a neuronal cell, or
a combination thereof.

18-20. (canceled)

21. The system of claim 17, wherein the factor is a growth factor.

22. The system of claim 21, wherein the growth factor comprises glial cell line-derived neurotrophic factor (GDNF), neurturin, growth/differentiation factor (GDF) 5, mesencephalic astrocyte-derived neurotrophic factor (MANF), cerebral dopaminergic neurotrophic factor (CDNF), or combinations thereof.

23. (canceled)

24. The system of claim 1, wherein the promoter-less donor vector comprises:
PGK polyadenylation signal (pA);
trimerized SV40pA;
the transgene or nucleic acid encoding an RNA;
loxP and flippase recognition target (FRT);
a rabbit beta-globin pA; and
a woodchuck hepatitis virus post-transcriptional regulatory element (WPRE).

25. A promoter-less donor vector, comprising:
a polyadenylation signal or transcription stop element upstream from a transgene or nucleic acid encoding an RNA;
the transgene or nucleic acid encoding an RNA; and
paired recombinase recognition sites.

26. The promoter-less donor vector of claim 25, wherein the promoter-less donor vector is selected from the group consisting of plasmid, viral vector, and bacterial artificial chromosome (BAC).

27. The promoter-less donor vector of claim 25, comprising at least four polyadenylation signals upstream from the transgene or nucleic acid encoding the RNA.

28. The promoter-less donor vector of claim 25, further comprising a post-transcriptional regulatory element, or further comprising a polyadenylation signal downstream from the transgene or nucleic acid encoding the RNA, or both.

29. (canceled)

30. The promoter-less donor vector of claim 25,
wherein the transgene or RNA is selected from the group consisting of an oncogene, loss-of-function (LOF) mutation of a tumor suppressor gene, gain-of-function (GOF) mutation of a proto-oncogene, pseudogene, siRNA, snRNA, sgRNA, lncRNA, miRNA, epigenetic modification, non-coding genetic or epigenetic abnormality associated with human disease, and combinations thereof, or
wherein the transgene comprises a factor that prevents apoptosis or promotes survival of a neuronal cell, increases the proliferation of a neuronal cell, or promotes differentiation of a neuronal cell.

31. (canceled)

32. The promoter-less donor vector of claim 30, wherein the factor is a growth factor.

33. The promoter-less donor vector of claim 32, wherein the growth factor comprises glial cell line-derived neurotrophic factor (GDNF), neurturin, growth/differentiation factor (GDF) 5, mesencephalic astrocyte-derived neurotrophic factor (MANF) or cerebral dopaminergic neurotrophic factor (CDNF), or combinations thereof.

34. (canceled)

35. The promoter-less donor vector of claim 25,
wherein one or both of the paired recombinase recognition sites comprise a mutation, or
wherein the viral vector is an adeno-associated viral (AAV) vector, or
wherein the mammalian cell comprises an embryonic stem cell, an adult stem cell, an induced pluripotent stem cell, or a tissue precursor cell, or
a combination thereof.

36. (canceled)

37. The promoter-less donor vector of claim 25, comprising:
PGK polyadenylation signal (pA);
trimerized SV40pA;
a transgene or nucleic acid encoding an RNA;
loxP and flippase recognition target (FRT);
a rabbit beta-globin pA; and
a woodchuck hepatitis virus post-transcriptional regulatory element (WPRE).

38. A method of genetic manipulation of a mammalian cell, comprising:
transfecting or transducing the mammalian cell with the system of claim 1.

39. The method of claim 38, wherein the mammalian cell is a human cell, the system targets an AAVS1 locus, H11 locus, or HPRT1 locus, and the method is an in vitro or ex vivo method, or wherein the mammalian cell is a mouse cell and the system targets a ROSA26 locus, Hipp11 locus, Tigre locus, ColA1 locus, or Hprt locus.

40. (canceled)

41. The method of claim 38, further comprising administering to the cell one or more recombinase enzymes.

42. The method of claim 40, wherein the one or more recombinase enzymes comprise, a Cre recombinase, a flippase recombinase, a Cre and a flippase recombinase, a Nigri recombinase, a Panto recombinase or a Vika recombinase.

43. (canceled)

44. A non-human animal model, comprising:
the non-human animal comprising a system of claim 1,
wherein the transgene or RNA is selected from the group consisting of an oncogene, loss-of-function (LOF) mutation of a tumor suppressor gene, gain-of-function (GOF) mutation of a proto-oncogene, pseudogene, siRNA, snRNA, sgRNA, lncRNA, miRNA, epigenetic modification, non-coding genetic or epigenetic abnormality associated with human disease, and combinations thereof.

45. The non-human animal model of claim 44,
wherein the non-human animal model is a personalized non-human animal model a human subject's cancer and the transgene or RNA is based on the human subject's cancer, or
wherein the non-human animal model is a personalized non-human animal model a human subject's disease or condition and the transgene or RNA is based on the human subject's disease or condition, or
wherein the non-human animal model comprises a gain of function mutation (GOF), a loss of function mutation (LOF), or both, or
a combination thereof.

46. (canceled)

47. (canceled)

48. A method of generating the non-human animal model of claim 44, comprising:
transfecting or transducing the non-human animal model with the system of claim 1,
wherein the transgene or RNA is selected from the group consisting of an oncogene, loss-of-function (LOF) mutation of a tumor suppressor gene, gain-of-function (GOF) mutation of a proto-oncogene, pseudogene, siRNA, snRNA, sgRNA, lncRNA, miRNA, epigenetic modification, non-coding genetic or epigenetic abnormality associated with human disease, and combinations thereof.

49. A method of assessing the effects of a drug candidate, comprising:
providing the non-human animal model of claim 44;
administering the drug candidate to the non-human animal model; and
assessing the effects of the drug candidate on the non-human animal model.

50. A mammalian cell comprising the system claim 1.

51. The cell of claim 50, wherein the cell is a human cell, or wherein the cell is a pluripotent cell.

52. (canceled)

53. The cell of claim 51, wherein the pluripotent cell is an induced pluripotent cell.

54. A method of delivering a gene product to an individual with a neurodegenerative disease or disorder comprising administering the mammalian cell of claim 50 to an individual in need thereof.

55. The method of claim 54, wherein the neurodegenerative disease or disorder comprises Parkinson's Disease, Amyotrophic Lateral Sclerosis (ALS), or Alzheimer's Disease.

56. (canceled)

57. (canceled)

58. A method of increasing a GDNF protein level in the brain of in an individual comprising administering the mammalian cell of claim 50 to the individual.

59. A mammalian cell comprising a genomic integrated transgene, wherein the genomic integrated transgene comprises a neurotrophic factor, and is integrated at a genomic site comprising a AAVS1 locus, H11 locus, or HPRT1 locus.

60. The mammalian cell of claim 59, wherein the cell is a human cell.

61. The mammalian cell of claim 60, wherein the human cell is an induced pluripotent stem cell.

62. The mammalian cell of claim 59,
wherein the neurotrophic factor comprises glial cell line-derived neurotrophic factor (GDNF), neurturin, growth/differentiation factor (GDF) 5, mesencephalic astrocyte-derived neurotrophic factor (MANF), cerebral dopaminergic neurotrophic factor (CDNF), or combinations thereof, or
wherein the neurotrophic factor is under the control of an inducible promoter, or both.

63. (canceled)

64. (canceled)

65. The mammalian cell of claim 62,
wherein the inducible promoter is a tetracycline inducible promoter,
wherein the neurotrophic factor and/or the inducible promoter are flanked by one or more of a recombinase recognition site, a tandem repeat of a transposable element, or an insulator sequence.

66. (canceled)

67. The mammalian cell of claim 65,
wherein the neurotropic factor and/or the inducible promoter are flanked by paired recombinase recognition sites, or
wherein the paired recombinase recognition sites comprise a variant recombinase recognition site and a wild-type recombinase recognition site, or
wherein the variant recombinase recognition site exhibits reduced cleavage by a recombinase compared to the wild-type recombinase recognition site, or
wherein the paired recombinase recognition sites comprise LoxP sites or FRT sites, or
a combination thereof.

68-70. (canceled)
